(19) United States
(12) Patent Application Publication
(10) Pub. No.: US 2025/0104876 A1
(43) Pub. Date: Mar. 27, 2025

(54) ANEUPLOIDY BIOMARKERS ASSOCIATED WITH RESPONSE TO ANTI-CANCER THERAPIES

(71) Applicant: Foundation Medicine, Inc., Boston, MA (US)

(72) Inventors: Kuei-Ting CHEN, Malden, MA (US); Ericka EBOT, Newton, MA (US); Radwa SHARAF, Medford, MA (US); Lee Alan ALBACKER, Arlington, MA (US)

(73) Assignee: Foundation Medicine, Inc., Boston, MA (US)

(21) Appl. No.: 18/905,014

(22) Filed: Oct. 2, 2024

Related U.S. Application Data
(63) Continuation of application No. PCT/US2023/017556, filed on Apr. 5, 2023.
(60) Provisional application No. 63/328,065, filed on Apr. 6, 2022.

(51) Int. Cl. G16H 50/30; G06N 20/00; G16B 20/10; G16B 20/20; G16H 10/60; G16H 20/10

(52) U.S. Cl. CPC G16H 50/30; G06N 20/00; G16B 20/10; G16B 20/20; G16H 10/60; G16H 20/10

(57) ABSTRACT

Methods for selecting an anti-cancer therapy for or treating a subject having cancer with an anti-cancer therapy comprising determining risk scores that predict the likelihood of response to the treatment are described. The methods may comprise, for example, obtaining genomic data comprising aneuploidy status or loss of heterozygosity data for one or more subgenomic intervals in a sample from the subject; analyzing the genomic data for the subject using a model configured to receive genomic data comprising aneuploidy status or loss of heterozygosity data for the one or more identified subgenomic intervals in the subject and output a risk score for the subject, wherein the risk score predicts the subject's response to a selected treatment. Also described are biomarkers for specific diseases (e.g., metastatic pancreatic cancer) and methods for treating subjects having cancer based on the determined risk scores.

### CROSS-REFERENCE TO RELATED APPLICATIONS

This application is a continuation application of International Application No. PCT/US2023/017556, filed internationally on Apr. 5, 2023, which claims the priority benefit of U.S. Provisional Patent Application Ser. No. 63/328,065, filed Apr. 6, 2022, the contents of each of which are incorporated herein by reference in their entirety.

### FIELD OF THE INVENTION

The present disclosure relates generally to methods for analyzing genomic profiling data, and more specifically to: (i) biomarkers (e.g., aneuploidy and/or loss of heterozygosity biomarkers) associated with response to an anti-cancer therapies, and (ii) methods of treating a subject using a risk score that predicts a response to a selected anti-cancer therapy using genomic profiling data.

### BACKGROUND

Pancreatic cancer accounts for about 3% of all cancers and is the fourth most common cause of cancer death in the United States. Current guidelines from the National Comprehensive Cancer Network (NCCN) for first-line treatment of patients with metastatic pancreatic cancer include treatment with either FOLFIRINOX or gemcitabine plus albumin-bound paclitaxel regimens. However, the efficacy of these two treatment regimens has not been compared in a randomized clinical trial, and as such, the decision to use one of these first line therapies over the other is challenging.

Thus, there is a need for predictive biomarkers of response to first line therapy in cancer patients, such as metastatic pancreatic cancer patients, that can facilitate decision-making by healthcare providers when selecting cancer therapies and treating cancer patients.

### BRIEF SUMMARY OF THE INVENTION

Provided herein are methods for treating a subject having cancer, or of selecting a treatment for a subject having cancer, comprising determining a risk score that predicts response to a first-line treatment for the cancer using genomic profiling data. Also disclosed herein are biomarkers (e.g., ancuploidy and/or loss of heterozygosity biomarkers) associated with a response to first-line treatments of cancer. In some instances, the biomarkers are specific chromosome arm-level aneuploidy events that are associated with survival of metastatic pancreatic cancer patients treated using either the FOLFIRINOX or gemcitabine plus albumin-bound paclitaxel (G+P) regimens.

In one aspect, provided herein are methods of treating a subject having a cancer with a first anti-cancer therapy comprising (a) determining a risk score for the subject based on ancuploidy status or loss of heterozygosity (LOH) for one or more subgenomic intervals in a sample from the subject, and (b) treating the subject with the first anti-cancer therapy if the risk score is less than a predetermined threshold.

In another aspect, provided herein are methods of selecting a first anti-cancer therapy for a subject having a cancer, the methods comprising determining a risk score for the subject based on ancuploidy status or loss of heterozygosity (LOH) for one or more subgenomic intervals in a sample from the subject, wherein if the risk score is less than a predetermined threshold, the subject is identified as one who may benefit from treatment with the first anti-cancer therapy.

In another aspect, provided herein are methods of identifying a subject having a cancer for treatment with a first anti-cancer therapy comprising (a) determining a risk score for the subject based on aneuploidy status or loss of heterozygosity (LOH) for one or more subgenomic intervals in a sample from the subject, and (b) identifying the subject for treatment with the first anti-cancer therapy if the risk score is less than a predetermined threshold.

In another aspect, provided herein are methods of identifying one or more treatment options for a subject having a cancer, the methods comprising (a) determining a risk score for the subject based on ancuploidy status or loss of heterozygosity (LOH) for one or more subgenomic intervals in a sample from the subject, and (b) generating a report comprising one or more treatment options identified for the subject based at least in part on the risk score determined for the sample, wherein if the risk score is less than a predetermined threshold, the subject is identified as one who may benefit from treatment with a first anti-cancer therapy.

In another aspect, provided herein are methods of predicting survival of a subject having cancer, the methods comprising determining a risk score for the subject based on aneuploidy status or loss of heterozygosity (LOH) for one or more subgenomic intervals in a sample from the subject, wherein if the risk score is less than a predetermined threshold, the subject is predicted to have longer survival when treated with a first anti-cancer therapy, as compared to a subject that is not treated with the first anti-cancer therapy.

In another aspect, provided herein are methods of monitoring, evaluating, or screening a subject having a cancer, the methods comprising determining a risk score for the subject based on aneuploidy status or loss of heterozygosity (LOH) for one or more subgenomic intervals in a sample from the subject, wherein if the risk score is less than a predetermined threshold, the subject is predicted to have longer survival when treated with a first anti-cancer therapy, as compared to a subject that was not treated with the first anti-cancer therapy.

In another aspect, provided herein are methods of predicting survival of a subject having cancer, the methods comprising determining a risk score for the subject based on aneuploidy status or loss of heterozygosity (LOH) for one or more subgenomic intervals in a sample from the subject, wherein if the risk score is less than a predetermined threshold, the subject is predicted to have longer survival when treated with a first anti-cancer therapy, as compared to a subject with a risk score that is greater than the predetermined threshold.

In another aspect, provided herein are methods of monitoring, evaluating, or screening a subject having a cancer, the methods comprising determining a risk score for the subject based on aneuploidy status or loss of heterozygosity (LOH) for one or more subgenomic intervals in a sample from the subject, wherein if the risk score is less than a predetermined threshold, the subject is predicted to have longer survival when treated with a first anti-cancer therapy, as compared to a subject with a risk score that is greater than the predetermined threshold.

In yet another aspect, provided herein are methods of stratifying a subject with cancer for treatment with a first anti-cancer therapy, the methods comprising determining a risk score for the subject based on aneuploidy status or loss of heterozygosity (LOH) for one or more subgenomic intervals in a sample from the subject, wherein if the risk score is less than a predetermined threshold, the subject is treated with the first anti-cancer therapy, and wherein if the risk score is more than the predetermined threshold, the subject is treated with a second anti-cancer therapy.

In some embodiments, the first anti-cancer therapy is a chemotherapy, an immune-oncology (IO) therapy, or a combination chemotherapy. In some embodiments, the IO therapy comprises a small molecule inhibitor, an antibody, a nucleic acid, an antibody-drug conjugate, a recombinant protein, a fusion protein, a natural compound, a peptide, a PROtcolysis-TArgeting Chimera (PROTAC), a cellular therapy, a treatment for cancer being tested in a clinical trial, an immunotherapy, or any combination thereof. In some embodiments, the combination chemotherapy comprises one or more of an alkylating agent, an alkyl sulfonates aziridine, an ethylenimine, a methylamelamine, an acctogenin, a camptothecin, a bryostatin, a callystatin, CC-1065, a cryptophycin, aa dolastatin, a duocarmycin, a cleutherobin, a pancratistatin, a sarcodictyin, a spongistatin, a nitrogen mustard, a nitrosureas, an antibiotic, a dynemicin, a bisphosphonate, an esperamicina a neocarzinostatin chromophore or a related chromoprotein enediyne antiobiotic chromophore, an anti-metabolite, a folic acid analogue, a purine analog, a pyrimidine analog, an androgens, an anti-adrenal, a folic acid replenisher, aldophosphamide glycoside, aminolevulinic acid, eniluracil, amsacrine, bestrabucil, bisantrene, edatraxate, defofamine, demecolcine, diaziquone, elformithine, elliptinium acetate, an cpothilone, ctoglucid, gallium nitrate, hydroxyurea, lentinan, lonidainine, maytansinoids, mitoguazone, mitoxantrone, mopidanmol, nitracrine, pentostatin, phenamet, pirarubicin, losoxantrone, podophyllinic acid, 2-ethylhydrazide, procarbazine, a PSK polysaccharide complex, razoxane, rhizoxin, sizofiran, spirogermanium, tenuazonic acid, triaziquone, 2,2′,2″-trichlorotriethylamine, a trichothecene, urethan, vindesine, dacarbazine, mannomustine, mitobronitol, mitolactol, pipobroman, gacytosine, arabinoside (“Ara-C”), cyclophosphamide, a taxoid, 6-thioguanine, mercaptopurine, a platinum coordination complex, vinblastine, platinum, etoposide (VP-16), ifosfamide, mitoxantrone, vincristine, vinorelbine, novantrone, teniposide, edatrexate, daunomycin, aminopterin, xeloda, ibandronate, irinotecan, topoisomerase inhibitor RFS 2000, difluorometlhylomithine (DMFO), a retinoid, capecitabine, carboplatin, procarbazine, plicomycin, gemcitabine, navelbine, farnesyl-protein transferase inhibitors, transplatinum, or any combination thereof. In some embodiments, the first anti-cancer therapy comprises FOLFIRINOX, gemcitabine plus albumin-bound paclitaxel, gemcitabine, capecitabine, fluorouacil plus irinotecan liposomal and leucovorin, FOLFIRI, or capecitabine plus gemcitabine. In some embodiments, the first anti-cancer therapy is FOLFIRINOX. In some embodiments, the first anti-cancer therapy is gemcitabine plus albumin-bound paclitaxel.

In another aspect, provided herein are methods of treating a subject having a cancer with gemcitabine plus albumin-bound paclitaxel comprising (a) determining a risk score for the subject based on aneuploidy status or loss of heterozygosity (LOH) for one or more subgenomic intervals in a sample from the subject, and (b) treating the subject with gemcitabine plus albumin-bound paclitaxel if the risk score is less than a predetermined threshold.

In another aspect, provided herein are methods of selecting a treatment for a subject having a cancer, the method comprising determining a risk score for the subject based on aneuploidy status or loss of heterozygosity (LOH) for one or more subgenomic intervals in a sample from the subject, wherein if the risk score is less than a predetermined threshold, the subject is identified as one who may benefit from treatment with gemcitabine plus albumin-bound paclitaxel.

In another aspect, provided herein are methods of identifying a subject having a cancer for treatment with gemcitabine plus albumin-bound paclitaxel comprising (a) determining a risk score for the subject based on aneuploidy status or loss of heterozygosity (LOH) for one or more subgenomic intervals in a sample from the subject, and (b) identifying the subject for treatment with gemcitabine plus albumin-bound paclitaxel if the risk score is less than a predetermined threshold.

In another aspect, provided herein are methods of identifying one or more treatment options for a subject having a cancer, the method comprising (a) determining a risk score for the subject based on ancuploidy status or loss of heterozygosity (LOH) for one or more subgenomic intervals in a sample from the subject, and (b) generating a report comprising one or more treatment options identified for the subject based at least in part on the risk score determined for the sample, wherein if the risk score is less than a predetermined threshold, the subject is identified as one who may benefit from treatment with gemcitabine plus albumin-bound paclitaxel.

In another aspect, provided herein are methods of predicting survival of a subject having cancer, the method comprising determining a risk score for the subject based on aneuploidy status or loss of heterozygosity (LOH) for one or more subgenomic intervals in a sample from the subject, wherein if the risk score is less than a predetermined threshold, the subject is predicted to have longer survival when treated with gemcitabine plus albumin-bound paclitaxel, as compared to a subject that is not treated with gemtabicine plus albumin-bound paclitaxel.

In another aspect, provided herein are methods of monitoring, evaluating, or screening an subject having a cancer, the method comprising determining a risk score for the subject based on aneuploidy status or loss of heterozygosity (LOH) for one or more subgenomic intervals in a sample from the subject, wherein if the risk score is less than a predetermined threshold, the subject is predicted to have longer survival when treated with gemcitabine plus albumin-bound paclitaxel, as compared to a subject that was not treated with gemcitabine plus albumin-bound paclitaxel.

In another aspect, provided herein are methods of predicting survival of a subject having cancer, the method comprising determining a risk score for the subject based on aneuploidy status or loss of heterozygosity (LOH) for one or more subgenomic intervals in a sample from the subject, wherein if the risk score is less than a predetermined threshold, the subject is predicted to have longer survival when treated with gemcitabine plus albumin-bound paclitaxel, as compared to a subject with a risk score that is greater than the predetermined threshold.

In another aspect, provided herein are methods of monitoring, evaluating, or screening a subject having a cancer, the method comprising determining a risk score for the subject based on aneuploidy status or loss of heterozygosity (LOH) for one or more subgenomic intervals in a sample from the subject, wherein if the risk score is less than a predetermined threshold, the subject is predicted to have longer survival when treated with gemcitabine plus albumin-bound paclitaxel, as compared to a subject with a risk score that is greater than the predetermined threshold.

In another aspect, provided herein are methods of stratifying a subject with cancer for treatment with gemcitabine plus albumin-bound paclitaxel, the method comprising determining a risk score for the subject based on aneuploidy status or loss of heterozygosity (LOH) for one or more subgenomic intervals in a sample from the subject, wherein if the risk score is less than a predetermined threshold, the subject is treated with with gemcitabine plus albumin-bound paclitaxel, and wherein if the risk score is more than the predetermined threshold, the subject is treated with a different anti-cancer therapy.

In another aspect, provided herein are methods of treating a subject having a cancer with FOLFIRINOX comprising (a) determining a risk score for the subject based on aneuploidy status or loss of heterozygosity (LOH) for one or more subgenomic intervals in a sample from the subject, and (b) treating the subject with FOLFIRINOX if the risk score is less than a predetermined threshold.

In another aspect, provided herein are methods of selecting a treatment for a subject having a cancer, the method comprising determining a risk score for the subject based on aneuploidy status or loss of heterozygosity (LOH) for one or more subgenomic intervals in a sample from the subject, wherein if the risk score is less than a predetermined threshold, the subject is identified as one who may benefit from treatment with FOLFIRINOX.

In another aspect, provided herein are methods of identifying a subject having a cancer for treatment with FOLFIRINOX comprising (a) determining a risk score for the subject based on aneuploidy status or loss of heterozygosity (LOH) for one or more subgenomic intervals in a sample from the subject, and (b) identifying the subject for treatment with FOLFIRINOX if the risk score is less than a predetermined threshold.

In another aspect, provided herein are methods of identifying one or more treatment options for a subject having a cancer, the method comprising (a) determining a risk score for the subject based on aneuploidy status or loss of heterozygosity (LOH) for one or more subgenomic intervals in a sample from the subject, and (b) generating a report comprising one or more treatment options identified for the subject based at least in part on the risk score determined for the sample, wherein if the risk score is less than a predetermined threshold, the subject is identified as one who may benefit from treatment with FOLFIRINOX.

In another aspect, provided herein are methods of predicting survival of a subject having cancer, the method comprising determining a risk score for the subject based on aneuploidy status or loss of heterozygosity (LOH) for one or more subgenomic intervals in a sample from the subject, wherein if the risk score is less than a predetermined threshold, the subject is predicted to have longer survival when treated with FOLFIRINOX, as compared to a subject that is not treated with FOLFIRINOX.

In another aspect, provided herein are methods of monitoring, evaluating, or screening an subject having a cancer, the method comprising determining a risk score for the subject based on aneuploidy status or loss of heterozygosity (LOH) for one or more subgenomic intervals in a sample from the subject, wherein if the risk score is less than a predetermined threshold, the subject is predicted to have longer survival when treated with FOLFIRINOX, as compared to a subject that was not treated with FOLFIRINOX.

In another aspect, provided herein are methods of predicting survival of a subject having cancer, the method comprising determining a risk score for the subject based on aneuploidy status or loss of heterozygosity (LOH) for one or more subgenomic intervals in a sample from the subject, wherein if the risk score is less than a predetermined threshold, the subject is predicted to have longer survival when treated with FOLFIRINOX, as compared to a subject with a risk score that is greater than the predetermined threshold.

In another aspect, provided herein are methods of monitoring, evaluating, or screening a subject having a cancer, the method comprising determining a risk score for the subject based on aneuploidy status or loss of heterozygosity (LOH) for one or more subgenomic intervals in a sample from the subject, wherein if the risk score is less than a predetermined threshold, the subject is predicted to have longer survival when treated with FOLFIRINOX, as compared to a subject with a risk score that is greater than the predetermined threshold.

In another aspect, provided herein are methods of stratifying a subject with cancer for treatment with FOLFIRINOX, the method comprising determining a risk score for the subject based on aneuploidy status or loss of heterozygosity (LOH) for one or more subgenomic intervals in a sample from the subject, wherein if the risk score is less than a predetermined threshold, the subject is treated with FOLFIRINOX, and wherein if the risk score is more than the predetermined threshold, the subject is treated with a different anti-cancer therapy.

In some embodiments of any of the above methods, risk score is calculated by a method comprising: obtaining genomic data comprising ancuploidy status or loss of heterozygosity data for one or more subgenomic intervals in a sample from the subject; and analyzing the genomic data for the subject using a model configured to receive genomic data comprising aneuploidy status or loss of heterozygosity data for the one or more identified subgenomic intervals in the subject and output a risk score for the subject, wherein the risk score predicts the subject's response to a selected treatment.

In some of any of the above methods, the method further comprises converting the risk score output by the model for the subject to a binary (high-low) risk score based on a comparison to a second predetermined threshold. In some embodiments, the predetermined threshold is defined as a mean, median, or mode of risk scores calculated for a patient cohort used to provide patient survival data used to train the model. In some embodiments, the predetermined threshold is defined by a risk score value that maximizes a log-rank statistic for risk scores calculated for a patient cohort used to provide patient survival data used to train the model. In some embodiments, a low risk score indicates that the subject is likely to survive longer than a subject with a high risk score if treated with the selected treatment.

In some embodiments, the model is a machine learning model. In some embodiments, the genomic data is based on sequence read data derived from a comprehensive genomic profiling assay. In some embodiments, analyzing the genomic data for the subject further comprises analysis of clinical feature data for the subject. In some embodiments, the clinical feature data comprises patient age, patient sex, patient race, patient clinical history, or any combination thereof. In some embodiments, analyzing the genomic data for the subject further comprises analysis of Eastern Cooperative Oncology Group (ECOG) performance data for the subject. In some embodiments, the machine learning model comprises a multivariable Cox proportional hazards regression model. In some embodiments, the machine learning model comprises a conditional inference forest model. In some embodiments, the one or more identified subgenomic intervals for which aneuploidy is correlated with a patient survival metric comprise chromosome arm-level aneuploidies. In some embodiments, the one or more identified subgenomic intervals for which LOH is correlated with a patient survival metric comprise chromosome arm-level aneuploidies. In some embodiments, the patient survival metric comprises a hazard ratio, a progression free survival, or any combination thereof.

In some embodiments, the risk score is for treatment with gemcitabine plus albumin-bound paclitaxel, and the one or more subgenomic intervals for which aneuploidy or LOH is correlated with a patient survival metric comprise Chr1p, Chr1q, Chr3p, Chr6p, Chr6q, Chr7p, Chr7q, Chr8q, Chr9p, Chr9q, Chr14q, Chr15q, Chr16p, Chr17p, Chr17q, Chr18q, Chr19p, Chr20p, Chr21p, Chr21q, Chr22q, or any combination thereof. In some embodiments, the risk score is for treatment with gemcitabine plus albumin-bound paclitaxel, and the one or more subgenomic intervals for which aneuploidy or LOH is correlated with a patient survival metric comprise Chr3p, Chr6p, Chr8q, Chr9q, Chr18q, Chr20p, Chr21p, Chr21q, Chr22q, or any combination thereof.

In some embodiments, the risk score is for treatment with FOLFIRINOX, and the one or more subgenomic intervals for which aneuploidy or LOH is correlated with a patient survival metric comprise Chr3q, Chr4p, Chr5p, Chr5q, Chr7q, Chr11p, Chr12p, Chr12q, Chr15q, Chr16p, Chr17p, Chr19p, Chr19q, Chr20p, Chr22q, or any combination thereof. In some embodiments, the risk score is for treatment with FOLFIRINOX, and the one or more subgenomic intervals for which aneuploidy or LOH is correlated with a patient survival metric comprise Chr7q, Chr15q, or any combination thereof.

In some embodiments of any of the above methods, the method further comprises treating the subject with gemcitabine plus albumin-bound paclitaxel.

In some embodiments of any of the above methods, the method further comprises treating the subject with FOLFIRINOX.

In some embodiments of any of the above methods, the method further comprises treating the subject with an additional anti-cancer therapy. In some embodiments, the anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, or any combination thereof.

In some embodiments of any of the above methods, the sample comprises a tissue biopsy sample or a liquid biopsy sample. In some embodiments, the sample is from a tumor biopsy, tumor specimen, or circulating tumor cell. In some embodiments, the sample is a liquid biopsy sample and comprises blood, plasma, cerebrospinal fluid, sputum, stool, urine, or saliva. In some embodiments, the sample comprises cells and/or nucleic acids from the cancer. In some embodiments, the sample comprises mRNA, DNA, circulating tumor DNA (ctDNA), cell-free DNA, cell-free RNA from the cancer, or any combination thereof. In some embodiments, the sample is a liquid biopsy sample and comprises circulating tumor cells (CTCs). In some embodiments, the sample is a liquid biopsy sample and comprises cell-free DNA (cfDNA), circulating tumor DNA (ctDNA), or any combination thereof.

In some embodiments of any of the above methods, the genomic data is based on sequence data derived from sequencing the sample from the subject. In some embodiments, the sequencing comprises use of a massively parallel sequencing (MPS) technique, whole genome sequencing (WGS), whole exome sequencing, targeted sequencing, direct sequencing, next-generation sequencing (NGS), or a Sanger sequencing technique. In some embodiments, the sequencing comprises: providing a plurality of nucleic acid molecules obtained from the sample, wherein the plurality of nucleic acid molecules comprises a mixture of tumor nucleic acid molecules and non-tumor nucleic acid molecules; optionally, ligating one or more adapters onto one or more nucleic acid molecules from the plurality of nucleic acid molecules; amplifying nucleic acid molecules from the plurality of nucleic acid molecules; optionally, capturing nucleic acid molecules from the amplified nucleic acid molecules, wherein the captured nucleic acid molecules are captured from the amplified nucleic acid molecules by hybridization to one or more bait molecules; and sequencing, by a sequencer, the captured nucleic acid molecules to obtain a plurality of sequence reads corresponding to one or more genomic loci within a subgenomic interval in the sample. In some embodiments, the adapters comprise one or more of amplification primer sequences, flow cell adapter hybridization sequences, unique molecular identifier sequences, substrate adapter sequences, or sample index sequences. In some embodiments, amplifying nucleic acid molecules comprises performing a polymerase chain reaction (PCR) technique, a non-PCR amplification technique, or an isothermal amplification technique. In some embodiments, the one or more bait molecules comprise one or more nucleic acid molecules, each comprising a region that is complementary to a region of a captured nucleic acid molecule. In some embodiments, the one or more bait molecules each comprise a capture moiety. In some embodiments, the capture moiety is biotin.

In some embodiments of any of the above methods, the cancer is a B cell cancer, a melanoma, breast cancer, lung cancer, bronchus cancer, colorectal cancer or carcinoma, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pincaloma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer or carcinoma, lung non-small cell lung carcinoma (NSCLC), head and neck cancer, small cell cancer, essential thrombocythemia, agnogenic myeloid metaplasia, hypercosinophilic syndrome, systemic mastocytosis, familiar hypereosinophilia, chronic eosinophilic leukemia, neuroendocrine cancers, or a carcinoid tumor. In some embodiments, the cancer is pancreatic cancer. In some embodiments, the pancreatic cancer is metastatic pancreatic cancer.

In some embodiments of any of the above methods, the subject is a human.

In some embodiments, the subject has previously been treated with an anti-cancer therapy. In some embodiments, the anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, or any combination thereof.

In some aspects, provided herein are also methods for determining a risk score for predicting subject response to a selected treatment for a disease. In some embodiments, the method comprises: receiving, at one or more processors, genomic data comprising aneuploidy status for one or more subgenomic intervals in each of a plurality of subjects exhibiting the disease who have been treated using the selected treatment; performing, using the one or more processors, a statistical analysis of the genomic data for the plurality of subjects to identify one or more subgenomic intervals for which aneuploidy is correlated with a subject survival metric for the selected treatment; and training, using the one or more processors, a model configured to receive genomic data comprising aneuploidy status for the one or more identified subgenomic intervals in a subject and output a risk score for the subject. In some embodiments, the risk score predicts a response to the selected treatment for the subject.

In some embodiments, the method further comprising determining a threshold for converting the risk score output by the machine learning model for the subject to a binary (high-low) risk score. In some embodiments, a low risk score indicates that the subject is likely to survive longer than a subject with a high risk score if treated with the selected treatment.

In some embodiments, the selected treatment is a selected first-line treatment for the disease.

In some embodiments, the genomic data is based on sequence read data derived from a comprehensive genomic profiling assay. In some embodiments, the genomic data further comprises loss of heterozygosity (LOH) data for the one or more subgenomic intervals in each of the plurality of subjects, and the statistical analysis further comprises identifying one or more subgenomic intervals for which LOH is correlated with the subject survival metric for the selected treatment. In some embodiments, the one or more identified subgenomic intervals for which aneuploidy is correlated with the subject survival metric comprise chromosome arm-level aneuploidies. In some embodiments, the one or more identified subgenomic intervals for which LOH is correlated with the subject survival metric comprise chromosome arm-level aneuploidies.

In some embodiments, the statistical analysis further comprises analysis of clinical feature data for the plurality of subjects. In some embodiments, the clinical feature data comprises subject age, subject sex, subject race, subject clinical history, or any combination thereof.

In some embodiments, the statistical analysis further comprises analysis of Eastern Cooperative Oncology Group (ECOG) performance data for the plurality of subjects.

In some embodiments, the model is a machine learning model. In some embodiments, the machine learning model comprises a multivariable Cox proportional hazards regression model. In some embodiments, the machine learning model comprises a conditional inference forest model.

In some embodiments the subject survival metric comprises a hazard ratio, a progression free survival, an overall survival, a disease-free survival, an objective tumor response rate, a time to tumor progression, a time to treatment failure, a durable complete response, a time to next treatment, or any combination thereof.

In some embodiments the disease is cancer. In some embodiments, the cancer is metastatic pancreatic cancer. In some embodiments, the subject is a human.

In some embodiments, the selected treatment comprises FOLFIRINOX or gemcitabine plus albumin-bound paclitaxel. In some embodiments, the selected treatment comprises gemcitabine plus albumin-bound paclitaxel, and the one or more subgenomic intervals for which aneuploidy or LOH is correlated with the subject survival metric comprise Chr3p, Chr6p, Chr8q, Chr9q, Chr18q, Chr20p, Chr21p, Chr21q, Chr22q, or any combination thereof. In some embodiments, the selected treatment comprisesFOLFIRINOX, and the one or more subgenomic intervals for which aneuploidy or LOH is correlated with the subject survival metric comprise Chr7q, Chr15q, or any combination thereof.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1 provides a non-limiting example of a process flowchart for determining a risk score that is predictive of a likely response to a selected treatment for a disease in a subject.

FIG. 2 provides a non-limiting example of a process flowchart for determining a combined risk score that predicts whether a subject will respond more favorably to a first selected treatment or a second selected treatment.

FIG. 3 provides a non-limiting example of a process flowchart for selecting a treatment and treating a subject patient according to the methods described herein.

FIG. 4 depicts an exemplary computing device or system in accordance with one embodiment of the present disclosure.

FIG. 5 depicts an exemplary computer system or computer network, in accordance with some instances of the systems described herein.

FIG. 6 provides a non-limiting example of a study design for determining risk scores for treatment of metastatic pancreatic cancer patients with either FOLFIRINOX or gemcitabine plus albumin-bound paclitaxel (G+P).

FIG. 7 provides a non-limiting example of data showing the clinical and treatment characteristics of a treatment cohort of metastatic pancreatic cancer patients. A chi-square test was used to calculate p values for categorical variables, while the Wilcoxon rank sum test was used to calculate p values for continuous variable (age at treatment start, and year at treatment).

FIG. 8 provides a non-limiting example of data showing the real-world survival of metastatic pancreatic cancer patients treated with first-line FOLF and G+P identified in a clinic-genomics database (CGDB). There was a significant difference in real-world survival when comparing the FOLF− and G+P-treated patients (p-value<0.001).

FIG. 9 provides a non-limiting example of data showing the association between clinical features and survival of metastatic pancreatic cancer patients treated with first-line FOLF and G+P as determined using univariate Cox regression models. As shown in the figure: ccogvalue_cat1, Eastern Cooperative Oncology Group (ECOG) score of 1; ecogvalue_cat2, ECOG score larger than or equal to 2; CA19_9_cat>59xULN, Cancer antigen 19-9 tumor marker test results higher than 2065; genderM, Gender: Male; age_trt, age treatment start; CA19_9_cat<59xULN, Cancer antigen 19-9 tumor marker test result between 35-2065; issurgery Yes, Surgery of Primary tumor: Yes; tissuecatpancreas, Tissue of origin: pancreas; and tissuecatother, Tissue of origin: other.

FIGS. 10A-10B provide a non-limiting example of data showing the association between chromosome arm level aneuploidies and survival of metastatic pancreatic cancer patients treated with first-line FOLF (FIG. 10A) or G+P (FIG. 10B). A univariate Cox proportional hazards model was used to calculate the hazard ratio (HR), and the p values were adjusted by the false discovery rate (FDR) method. The horizontal dash line indicate adjusted p value≤0.05. Among the FOLF-treated cohort, two ancuploidy features were identified as associated with survival (adjusted p<0.05). Among the G+P treated cohort, fourteen ancuploidy features were associated with survival (adjusted p<0.05). As shown in the figure: HR, hazard ratio; and freq, frequency in patient cohort.

FIGS. 11A-11B provide a non-limiting example of data showing the association between chromosome arm level aneuploidies and survival of metastatic pancreatic cancer patients treated with first-line FOLF (FIG. 11A) or G+P (FIG. 11B). A univariate Cox proportional hazards model was used to calculate the hazard ratio (HR). The horizontal dash line indicates p value≤ 0.05. Among the FOLF-treated cohort, fifteen aneuploidy features were identified as associated with survival (p<0.05). Among the G+P treated cohort, twenty-one aneuploidy features were associated with survival (p<0.05). As shown in the figure: HR, hazard ratio; and freq, frequency in patient cohort.

FIGS. 12A-12D provide a non-limiting example of data showing the prevalence and association of chromosome arm level aneuploidies with survival in metastatic pancreatic cancer patients treated with first-line FOLF or G+P. FIGS. 12A-12C show the prevalence of aneuploidy features across chromosome arm for each treatment group. The aneuploidy features were classified as gain (FIG. 12A), loss (FIG. 12B) and loss of heterozygosity (LOH; FIG. 12C). FIG. 12D shows the association of chromosome arm level ancuploidies and survival of metastatic pancreatic cancer patients treated with first-line FOLF or G+P as determined using a univariate Cox regression model. As shown in the figure: HR, hazard ratio.

FIG. 13 provides a non-limiting example of data showing the hazard ratio (HR) corresponding to chromosome arm level aneuploidies and clinical features associated with survival in metastatic pancreatic cancer patients treated with first-line FOLF.

FIGS. 14A-14B provide a non-limiting example of data showing the association between a FOLF risk score and metastatic pancreatic cancer patient survival based on training (FIG. 14A) and testing (FIG. 14B) datasets. The FOLF risk score was determined using a multivariate Cox regression model trained on data for chromosome arm level aneuploidies and clinical features in the FOLF-treated patient cohort. A cut-off value for FOLF risk score stratification was defined based on the median linear predictor in the training data, and patients were then stratified as FOLF risk score high or low for the survival analysis. The lower panel in each figure indicates the number of patients at risk versus months of treatment stratified by FOLF risk score. As shown in the figure: score=high, FOLF risk score high; score=low, FOLF risk score low; and time, time in months.

FIG. 15 provides a non-limiting example of data showing the hazard ratio (HR) corresponding to chromosome arm level aneuploidies and clinical features associated with survival in metastatic pancreatic cancer patients treated with first-line G+P.

FIGS. 16A-16B provide a non-limiting example of data showing the association between a G+P risk score and metastatic pancreatic cancer patient survival based on training (FIG. 16A) and testing (FIG. 16B) datasets. The G+P risk score was determined using a multivariate Cox regression model trained on data for chromosome arm level aneuploidies and clinical features in the G+P-treated patient cohort. A cut-off value for G+P risk score stratification was defined based on the median linear predictor in the training data, and patients were then stratified as FOLF risk score high or low for the survival analysis. The G+P risk score was associated with patient survival in the testing dataset. The lower panel in each figure indicates the number of patients at risk versus months of treatment stratified by G+P risk score. As shown in the figure: score=high, G+P risk score high; score=low, G+P risk score low; and time, time in months.

FIG. 17 provides a non-limiting example of data showing the hazard ratio (HR) corresponding to chromosome arm level aneuploidies associated with survival in metastatic pancreatic cancer patients treated with first-line FOLF.

FIGS. 18A-18B provide a non-limiting example of data showing the association between a FOLF risk score and metastatic pancreatic cancer patient survival based on training (FIG. 18A) and testing (FIG. 18B) datasets. The FOLF risk score was determined using a multivariate Cox regression model trained only on data for chromosome arm level aneuploidies from the FOLF-treated patient cohort. A cut-off value for FOLF risk score stratification was defined based on the median linear predictor in the training data, and patients were then stratified as FOLF risk score high or low for the survival analysis. The lower panel in each figure indicates the number of patients at risk versus months of treatment stratified by FOLF risk score. As shown in the figure: score=high, FOLF risk score high; score=low, FOLF risk score low; and time, time in months.

FIG. 19 provides a non-limiting example of data showing a max log rank statistical analysis of a FOLF risk score determined based on chromosome arm level aneuploidy data associated with metastatic pancreatic cancer patient survival. A cut-off value for FOLF risk score stratification was defined based on the linear predictor value that maximized the log rank statistic in the training dataset. The linear predictor risk score was calculated from a multivariate Cox model. The arrow indicates the linear predictor value that maximizes the log rank statistic in the training dataset and the linear predictor is used to stratify the binary FOLF risk score. As shown in the figure: lp, linear predictor.

FIGS. 20A-20B provide a non-limiting example of data showing the association between a FOLF risk score and metastatic pancreatic cancer patient survival based on training (FIG. 20A) and testing (FIG. 20B) datasets. The FOLF risk score was determined using a multivariate Cox regression model trained only on data for chromosome arm level aneuploidies from the FOLF-treated patient cohort. A cut-off value for G+P risk score stratification was defined based on the median linear predictor in the training data, and patients were then stratified as G+P risk score high or low for the survival analysis. FOLF-treated patients with a high FOLF risk score had worse survival compared to those with a low FOLF risk score in the training dataset (HR=5.11, CI=3.14-8.33, p=<0.001). The lower panel in each figure indicates the number of patients at risk versus months of treatment stratified by FOLF risk score. As shown in the figure: score=high, FOLF risk score high; score=low, FOLF risk score low; and time, time in months.

FIG. 21 provides a non-limiting example of data showing the hazard ratio (HR) corresponding to chromosome arm level aneuploidies associated with survival in metastatic pancreatic cancer patients treated with first-line G+P.

FIGS. 22A-22B provide a non-limiting example of data showing the association between a G+P risk score and metastatic pancreatic cancer patient survival based on training (FIG. 22A) and testing (FIG. 22B) datasets. The G+P risk score was determined using a multivariate Cox regression model trained only on data for chromosome arm level aneuploidies from the G+P-treated patient cohort. A cut-off value for G+P risk score stratification was defined based on the linear predictor risk score that maximized a max log rank statistic for the training dataset, and patients were then stratified as G+P risk score high or low for the survival analysis. G+P-treated patients with a high G+P risk score had worse survival compared to those with a low G+P risk score in the training dataset (HR=2.06, CI=1.62-2.61, p=<0.001). This association remained among G+P-treated patient in test dataset (HR=2.40, CI=1.46-3.93, p=<0.001). The lower panel in each figure indicates the number of patients at risk versus months of treatment stratified by G+P risk score. As shown in the figure: score=high, G+P risk score high; score=low, G+PF risk score low; and time, time in months.

FIG. 23 provides a non-limiting example of data showing a max log rank statistical analysis of a G+P risk score determined based on chromosome arm level ancuploidy data associated with metastatic pancreatic cancer patient survival. A cut-off value for G+P risk score stratification was defined based on the linear predictor value that maximized the log rank statistic for the training dataset. The linear predictor risk score was calculated from a multivariate Cox model. The arrow indicates the linear predictor value that maximizes the log rank statistic in the training dataset and the linear predictor is used to stratify the binary G+P risk score. As shown in the figure: lp, linear predictor.

FIGS. 24A-24B provide a non-limiting example of data showing the association between a G+P risk score and metastatic pancreatic cancer patient survival based on training (FIG. 24A) and testing (FIG. 24B) datasets. The G+P risk score was determined using a multivariate Cox regression model trained only on data for chromosome arm level ancuploidies from the G+P-treated patient cohort. A cut-off value for G+P risk score stratification was defined based on the linear predictor value that maximized the log rank statistic for the training dataset, and patients were then stratified as G+P risk score high or low for the survival analysis. The lower panel in each figure indicates the number of patients at risk versus months of treatment stratified by G+P risk score. As shown in the figure: score=high, G+P risk score high; score=low, G+P risk score low; and time, time in months.

FIG. 25 provides a non-limiting example of data showing a forest plot depicting the hazard ratio (HR) of FOLF or G+P risk scores for patients treated with either FOLF or G+P. The p-value indicates the significance of the interaction between risk score and survival on the treatment. FOLF risk scores were associated with survival (p<0.001) in FOLF-treated patients, but not amongst G+P-treated patients (p=0.59). A significant interaction was observed between FOLF risk score and treatment (p<0.001). G+P risk scores were associated with survival in G+P treated patients (p<0.001), but not amongst FOLF-treated patient (p=0.41). A significant interaction was observed between G+P risk score and treatment (p=0.01).

FIG. 26 provides a non-limiting example of data showing a forest plot depicting a multivariate analysis of the association of G+P risk scores and clinical factors with patient survival on the treatment. G+P risk scores were significantly associated with survival, even after adjusting for clinical features (HR=2.87, CI=1.53-5.37, p=0.001). As shown in the figure: Risk_score_GP, G+P risk score value; ecogvalue_cat1, Eastern Cooperative Oncology Group (ECOG) score equal to 1; CA19_9_cat, Cancer antogen 19-9 tumor marker test result; gender, Gender; issurgery, Surgery of Primary tumor; tissuecat, Tissue of origin; and primarysite, Primary tumor site.

FIG. 27 provides a non-limiting example of data showing the integrated Brier score distribution for the conditional inference survival forest (CIF) or multivariate Cox regression model used to determine FOLF and G+P risk scores. The risk scores were determined based on chromosome arm level aneuploidy data only, and subjected to 5-fold cross-validation. The FOLF and G+P risk scores determined using a CIF model outperform those determined using a univariate Cox regression model. As shown in the figure: cif_folfirinox, FOLF risk scores determined using CIF model; cif_gp, G+P risk scores determined using CIF model, cox_folfirinox, FOLF risk scores determined using univariate Cox regression model; cox_gp, G+P risk scores determined using univariate Cox regression model; and IBS, integrated Brier score.

FIG. 28 provides a non-limiting example of data showing the mean Brier scores per month of treatment for FOLF or G+P risk scores determined using a conditional inference survival forest (CIF) or an univariate Cox regression model. The risk scores were determined based on chromosome arm level aneuploidy data only, and subjected to 5-fold cross-validation. At all timepoints, the FOLF and G+P risk scores determined using a CIF model outperform those determined using a univariate Cox regression model. The predictive accuracy of the risk scores was found to decrease starting from 6 to 18 months of treatment for scores determined with either model. As shown in the figure: cif_folfirinox, FOLF risk scores determined using CIF model; cif_gp, G+P risk scores determined using CIF model, cox_folfirinox, FOLF risk scores determined using univariate Cox regression model; cox_gp, G+P risk scores determined using univariate Cox regression model; and month, time in months.

FIGS. 29A-29B provides a non-limiting example of data showing the distribution of variable importance scores for FOLF (FIG. 29A) or G+P (FIG. 29B) risk scores determined using a CIF model. Variable importance scores were calculated for each identified chromosome arm level aneuploidy. Boxes indicate chromosomes arms with an adjusted p-value<0.05 when using a univariate cox regression model to determine the risk score. As shown in the figure: Arm, chromosome arm level aneuploidy; and imp, importance score.

FIG. 30 provides a non-limiting example of a study design for identifying associations between cytoband level aneuploidy data and patient survival in a metastatic pancreatic cancer patient cohort treated with gemcitabine plus albumin-bound paclitaxel (G+P, also referred to as GP).

FIGS. 31A-31B provide non-limiting examples of plots of adjusted p value versus hazard ratio (HR) for cohorts of metastatic pancreatic cancer patient treated with either FOLF or G+P that demonstrate associations between chromosome arm level ancuploidy data and survival in each cohort. FIG. 31A: data for the FOLF cohort. FIG. 31B: data for the G+P cohort.

FIGS. 32A-32D provide non-limiting examples of plots of copy number gain, copy number loss, and loss of heterozygosity (Loh) data that demonstrate associations between cytoband level aneuploidy data and survival in a GP-treated cohort of metastatic pancreatic cancer patients. FIG. 32A: copy number gain data. FIG. 32B: copy number loss data. FIG. 32C: loss of heterozygosity (Loh) data. FIG. 32D: summary of chromosome regions that may exhibit chromosome alterations comprising loss of heterozygosity.

FIGS. 33A-33C provide non-limiting examples of plots of copy number gain, copy number loss, and loss of heterozygosity (Loh) data that demonstrate associations between cytoband level aneuploidy data and survival in a FOLF-treated cohort of metastatic pancreatic cancer patients. FIG. 33A: copy number gain data. FIG. 33B: copy number loss data. FIG. 33C: loss of heterozygosity (Loh) data.

FIG. 34 provides a non-limiting example of hazard ratio (HR) data plotted for loss of heterozygosity in different regions of chromosome 3 for a cohort of metastatic pancreatic cancer patients treated with G+P.

FIGS. 35A-35B provide non-limiting examples of survival plots for a cohort of metastatic pancreatic cancer patients treated with G+P. FIG. 35A: survival data for metastatic pancreatic patients exhibiting deletions at chromosome region 3.p11.2 compared to that for patients with no deletion at 3.p11.2. FIG. 35B: survival data for metastatic pancreatic patients exhibiting deletions at chromosome region 3.p25.1 compared to that for patients with no deletion at 3.p25.1.

FIG. 36 provides a non-limiting example of hazard ratio (HR) data plotted for loss of heterozygosity in different regions of chromosome 6 for a cohort of metastatic pancreatic cancer patients treated with G+P.

FIG. 37 provides a non-limiting example of a survival plot for a cohort of metastatic pancreatic cancer patients treated with G+P. The plot shows survival data for metastatic pancreatic patients exhibiting deletions at chromosome region 6.p22.2 compared to that for patients with no deletion at 6.p22.2.

FIG. 38 provides a non-limiting example of hazard ratio (HR) data plotted for copy number loss in different regions of chromosome 21 for a cohort of metastatic pancreatic cancer patients treated with G+P.

FIG. 39 provides a non-limiting example of a survival plot for a cohort of metastatic pancreatic cancer patients treated with G+P. The plot shows survival data for metastatic pancreatic patients exhibiting copy number loss at chromosome region 21.q22.12 compared to that for patients with no copy number loss at 21.q22.12.

### DETAILED DESCRIPTION

Provided herein are methods of selecting a first anti-cancer therapy treatment or treatment regimen (e.g., FOLFIRINOX or gemcitabine plus albumin-bound paclitaxel) for a subject having a cancer. In some instances, the method comprises determining a risk score for the subject based on ancuploidy status or loss of heterozygosity (LOH) for one or more subgenomic intervals in a sample from the subject, wherein if the risk score is less than a predetermined threshold, the subject is identified as one who may benefit from treatment with the first anti-cancer therapy or treatment regimen (e.g., FOLFIRINOX or gemcitabine plus albumin-bound paclitaxel). In some instances, the method comprises identifying the subject for as one who may benefit from treatment with, e.g., FOLFIRINOX if the risk score is less than a predetermined threshold, e.g., a first predetermined threshold. In some instances, the method comprises identifying the subject as one who may benefit from treatment with, e.g., gemcitabine plus albumin-bound paclitaxel if the risk score is less than a predetermined threshold, e.g., a second predetermined threshold.

Also herein are methods of treating a subject having cancer with a first anti-cancer therapy treatment or treatment regimen (e.g., FOLFIRINOX or gemcitabine plus albumin-bound paclitaxel). In some instances, the method comprises determining a risk score for the subject based on aneuploidy status or loss of heterozygosity (LOH) for one or more subgenomic intervals in a sample from the subject, wherein if the risk score is less than a predetermined threshold, the subject is treated with the first anti-cancer therapy (e.g., FOLFIRINOX or gemcitabine plus albumin-bound paclitaxel). In some instances, the method comprises treating the subject with, e.g., FOLFIRINOX if the risk score is less than a predetermined threshold, e.g., a first predetermined threshold. In some instances, the method comprises treating the subject with, e.g., gemcitabine plus albumin-bound paclitaxel if the risk score is less than a predetermined threshold, e.g., a second predetermined threshold.

In some instances, for example, methods comprise determining a risk score by obtaining genomic data comprising aneuploidy status or loss of heterozygosity data for one or more subgenomic intervals in a sample from the subject; and analyzing the genomic data for the subject using a machine learning model configured to receive genomic data comprising aneuploidy status or loss of heterozygosity data for the one or more identified subgenomic intervals in the subject and output a risk score for the subject, wherein the risk score predicts the subject's response to a selected treatment.

In some instances, the method may further comprise converting the risk score output by the machine learning model to a binary (high-low) risk score based on a comparison to a second predetermined threshold, where, for example, a low risk score indicates that the subject is likely to survive longer than a subject with a high risk score if treated with the first line therapy.

In some instances, the genomic data further comprises loss of heterozygosity (LOH) data for the one or more subgenomic intervals in the subject, and analyzing the genomic data further comprises identifying one or more subgenomic intervals for which LOH is correlated with the patient survival metric for the first line therapy. In some instances, analyzing the genomic data further comprises analysis of clinical feature data for the subject (e.g., patient age, patient sex, patient race, patient clinical history, or any combination thereof). In some instances, analyzing the genomic data further comprises analysis of Eastern Cooperative Oncology Group (ECOG) performance data for the subject.

The disclosed methods provide improved decision-making tools to help healthcare providers choose which of the available first line treatment options for a cancer is likely to be most effective for a specific subject.

Also provided herein are specific chromosome arm-level aneuploidy biomarkers that are associated with survival of metastatic pancreatic cancer patients treated using either the FOLFIRINOX or gemcitabine plus albumin-bound paclitaxel (G+P) regimens.

### I. Definitions

Unless otherwise defined, all of the technical terms used herein have the same meaning as commonly understood by one of ordinary skill in the art in the field to which this disclosure belongs.

As used in this specification and the appended claims, the singular forms “a”, “an”, and “the” include plural references unless the context clearly dictates otherwise. Any reference to “or” herein is intended to encompass “and/or” unless otherwise stated.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, un-recited additives, components, integers, elements, or method steps.

The terms “cancer” and “tumor” are used interchangeably herein. These terms refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell. These terms include a solid tumor, a soft tissue tumor, or a metastatic lesion. As used herein, the term “cancer” includes premalignant, as well as malignant cancers.

“Polynucleotide,” “nucleic acid,” or “nucleic acid molecule” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. Thus, for instance, polynucleotides as defined herein include, without limitation, single— and double-stranded DNA, DNA including single—and double-stranded regions, single— and double-stranded RNA, and RNA including single—and double-stranded regions, hybrid molecules comprising DNA and RNA that may be single-stranded or, more typically, double-stranded or include single— and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes cDNAs.

A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps,” substitution of one or more of the naturally-occurring nucleotides with an analog, internucleotide modifications such as, for example, those with uncharged linkages (e.g., methyl phosphonates, phosphotriesters, phosphoamidates, carbamates, and the like) and with charged linkages (e.g., phosphorothioates, phosphorodithioates, and the like), those containing pendant moieties, such as, for example, proteins (e.g., nucleases, toxins, antibodies, signal peptides, poly-L-lysine, and the like), those with intercalators (e.g., acridine, psoralen, and the like), those containing chelators (e.g., metals, radioactive metals, boron, oxidative metals, and the like), those containing alkylators, those with modified linkages (e.g., alpha anomeric nucleic acids), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl-, 2′-fluoro-, or 2′-azido-ribose, carbocyclic sugar analogs, a-anomeric sugars, epimeric sugars such as arabinose, xyloses or lyxoses, pyranose sugars, furanose sugars, sedoheptuloses, acyclic analogs, and abasic nucleoside analogs such as methyl riboside. One or more phosphodiester linkages may be replaced by alternative linking groups. These alternative linking groups include, but are not limited to, instances wherein phosphate is replaced by P(0)S (“thioate”), P(S) S (“dithioate”), “(0) NR₂ (“amidate”), P(0)R, P (O) OR′, CO or CH₂ (“formacetal”), in which each R or R′is independently H or substituted or unsubstituted alkyl (1-20° C.) optionally containing an ether (-0-) linkage, aryl, alkenyl, cycloalkyl, cycloalkenyl or araldyl. Not all linkages in a polynucleotide need be identical. A polynucleotide can contain one or more different types of modifications as described herein and/or multiple modifications of the same type. The preceding description applies to all polynucleotides referred to herein, including RNA and DNA.

The term “detection” includes any means of detecting, including direct and indirect detection. The term “biomarker” as used herein refers to an indicator, e.g., predictive, diagnostic, and/or prognostic, which can be detected in a sample. The biomarker may serve as an indicator of a particular subtype of a disease or disorder (e.g., cancer) characterized by certain, molecular, pathological, histological, and/or clinical features (e.g., responsiveness to therapy, e.g., a checkpoint inhibitor). In some instances, a biomarker is a collection of genes or a collective number of mutations/alterations (e.g., somatic mutations) in a collection of genes. Biomarkers include, but are not limited to, polynucleotides (e.g., DNA and/or RNA), polynucleotide alterations (e.g., polynucleotide copy number alterations, e.g., DNA copy number alterations, or other mutations or alterations), polypeptides, polypeptide and polynucleotide modifications (e.g., post-translational modifications), carbohydrates, and/or glycolipid-based molecular markers.

“Amplification,” as used herein generally refers to the process of producing multiple copies of a desired sequence. “Multiple copies” mean at least two copies. A “copy” does not necessarily mean perfect sequence complementarity or identity to the template sequence. For example, copies can include nucleotide analogs such as deoxyinosine, intentional sequence alterations (such as sequence alterations introduced through a primer comprising a sequence that is hybridizable, but not complementary, to the template), and/or sequence errors that occur during amplification.

The technique of “polymerase chain reaction” or “PCR” as used herein generally refers to a procedure wherein minute amounts of a specific piece of nucleic acid, RNA and/or DNA, are amplified as described, for example, in U.S. Pat. No. 4,683,195. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage, or plasmid sequences, etc. Sec generally Mullis et al., Cold Spring Harbor Symp. Quant. Biol. 51:263 (1987) and Erlich, ed., PCR Technology (Stockton Press, NY, 1989). As used herein, PCR is considered to be one, but not the only, example of a nucleic acid polymerase reaction method for amplifying a nucleic acid test sample, comprising the use of a known nucleic acid (DNA or RNA) as a primer and utilizes a nucleic acid polymerase to amplify or generate a specific piece of nucleic acid or to amplify or generate a specific piece of nucleic acid which is complementary to a particular nucleic acid.

“Subject response” or “response” can be assessed using any endpoint indicating a benefit to the subject, including, without limitation, (1) inhibition, to some extent, of disease progression (e.g., cancer progression), including slowing down or complete arrest; (2) a reduction in tumor size; (3) inhibition (i.e., reduction, slowing down, or complete stopping) of cancer cell infiltration into adjacent peripheral organs and/or tissues; (4) inhibition (i.e. reduction, slowing down, or complete stopping) of metastasis; (5) relief, to some extent, of one or more symptoms associated with the disease or disorder (e.g., cancer); (6) increase or extension in the length of survival, including overall survival and progression free survival; and/or (7) decreased mortality at a given point of time following treatment.

An “effective response” of a subject or a subject's “responsiveness” to treatment with a medicament and similar wording refers to the clinical or therapeutic benefit imparted to a subject at risk for, or suffering from, a disease or disorder, such as cancer. In one instance, such benefit includes any one or more of: extending survival (including overall survival and/or progression-free survival); resulting in an objective response (including a complete response or a partial response); or improving signs or symptoms of cancer.

As used herein, “treatment” (and grammatical variations thereof such as “treat” or “treating”) refers to clinical intervention in an attempt to alter the natural course of the subject being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the terms “individual,” “patient,” or “subject” are used interchangeably and refer to any single animal, e.g., a mammal (including such non-human animals as, for example, dogs, cats, horses, rabbits, zoo animals, cows, pigs, sheep, and non-human primates) for which treatment is desired. In particular instances, the subject herein is a human.

As used herein, “administering” is meant a method of giving a dosage of an agent or a pharmaceutical composition (e.g., a pharmaceutical composition including the agent) to a subject. Administering can be by any suitable means, including parenteral, intrapulmonary, and intranasal, and, if desired for local treatment, intralesional administration. Parenteral infusions include, for example, intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

The terms “concurrently” or “in combination” are used herein to refer to administration of two or more therapeutic agents, where at least part of the administration overlaps in time. Accordingly, concurrent administration includes a dosing regimen when the administration of one or more agent(s) continues after discontinuing the administration of one or more other agent(s).

“Acquire” or “acquiring” as the terms are used herein, refer to obtaining possession of a physical entity, or a value, e.g., a numerical value, by “directly acquiring” or “indirectly acquiring” the physical entity or value. “Directly acquiring” means performing a process (e.g., performing a synthetic or analytical method) to obtain the physical entity or value. “Indirectly acquiring” refers to receiving the physical entity or value from another party or source (e.g., a third-party laboratory that directly acquired the physical entity or value). Directly acquiring a physical entity includes performing a process that includes a physical change in a physical substance, e.g., a starting material. Exemplary changes include making a physical entity from two or more starting materials, shearing or fragmenting a substance, separating or purifying a substance, combining two or more separate entities into a mixture, performing a chemical reaction that includes breaking or forming a covalent or non-covalent bond. Directly acquiring a value includes performing a process that includes a physical change in a sample or another substance, e.g., performing an analytical process which includes a physical change in a substance, e.g., a sample, analyte, or reagent (sometimes referred to herein as “physical analysis”), performing an analytical method, e.g., a method which includes one or more of the following: separating or purifying a substance, e.g., an analyte, or a fragment or other derivative thereof, from another substance; combining an analyte, or fragment or other derivative thereof, with another substance, e.g., a buffer, solvent, or reactant; or changing the structure of an analyte, or a fragment or other derivative thereof, e.g., by breaking or forming a covalent or non-covalent bond, between a first and a second atom of the analyte; or by changing the structure of a reagent, or a fragment or other derivative thereof, e.g., by breaking or forming a covalent or non-covalent bond, between a first and a second atom of the reagent.

“Acquiring a sequence” or “acquiring a read” as the term is used herein, refers to obtaining possession of a nucleotide sequence or amino acid sequence, by “directly acquiring” or “indirectly acquiring” the sequence or read. “Directly acquiring” a sequence or read means performing a process (e.g., performing a synthetic or analytical method) to obtain the sequence, such as performing a sequencing method (e.g., a Next-generation Sequencing (NGS) method). “Indirectly acquiring” a sequence or read refers to receiving information or knowledge of, or receiving, the sequence from another party or source (e.g., a third-party laboratory that directly acquired the sequence). The sequence or read acquired need not be a full sequence, e.g., sequencing of at least one nucleotide, or obtaining information or knowledge, that identifies one or more of the alterations disclosed herein as being present in a sample, biopsy or subject constitutes acquiring a sequence.

Directly acquiring a sequence or read includes performing a process that includes a physical change in a physical substance, e.g., a starting material, such as a sample described herein. Exemplary changes include making a physical entity from two or more starting materials, shearing or fragmenting a substance, such as a genomic DNA fragment; separating or purifying a substance (e.g., isolating a nucleic acid sample from a tissue); combining two or more separate entities into a mixture, performing a chemical reaction that includes breaking or forming a covalent or non-covalent bond. Directly acquiring a value includes performing a process that includes a physical change in a sample or another substance as described above. The size of the fragment (e.g., the average size of the fragments) can be 2500 bp or less, 2000 bp or less, 1500 bp or less, 1000 bp or less, 800 bp or less, 600 bp or less, 400 bp or less, or 200 bp or less. In some instances, the size of the fragment (e.g., cfDNA) is between about 150 bp and about 200 bp (e.g., between about 160 bp and about 170 bp). In some instances, the size of the fragment (e.g., DNA fragments from liquid biopsy samples) is between about 150 bp and about 250 bp. In some instances, the size of the fragment (e.g., cDNA fragments obtained from RNA in liquid biopsy samples) is between about 100 bp and about 150 bp.

As used herein, the term “subgenomic interval” (or “subgenomic sequence interval”) refers to a portion of a genomic sequence.

As used herein, the term “subject interval” refers to a subgenomic interval or an expressed subgenomic interval (e.g., the transcribed sequence of a subgenomic interval).

“Alteration” or “altered structure” as used herein, of a gene or gene product (e.g., a marker gene or gene product) refers to the presence of a mutation or mutations within the gene or gene product, e.g., a mutation, which affects integrity, sequence, structure, amount or activity of the gene or gene product, as compared to the normal or wild-type gene. The alteration can be in amount, structure, and/or activity in a cancer tissue or cancer cell, as compared to its amount, structure, and/or activity, in a normal or healthy tissue or cell (e.g., a control), and is associated with a disease state, such as cancer. For example, an alteration which is associated with cancer, or predictive of responsiveness to anti-cancer therapeutics, can have an altered nucleotide sequence (e.g., a mutation), amino acid sequence, chromosomal translocation, intra-chromosomal inversion, copy number, expression level, protein level, protein activity, epigenetic modification (e.g., methylation or acetylation status, or post-translational modification, in a cancer tissue or cancer cell, as compared to a normal, healthy tissue or cell. Exemplary mutations include, but are not limited to, point mutations (e.g., silent, missense, or nonsense), deletions, insertions, inversions, duplications, amplification, translocations, inter— and intra-chromosomal rearrangements. Mutations can be present in the coding or non-coding region of the gene. In certain instances, the alteration(s) is detected as a rearrangement, e.g., a genomic rearrangement comprising one or more introns or fragments thereof (e.g., one or more rearrangements in 5′—and/or 3′-UTR). In certain instances, the alterations are associated (or not associated) with a phenotype, e.g., a cancerous phenotype (e.g., one or more of cancer risk, cancer progression, cancer treatment or resistance to cancer treatment). In one instance, the alteration (or tumor mutational burden) is associated with one or more of: a genetic risk factor for cancer, a positive treatment response predictor, a negative treatment response predictor, a positive prognostic factor, a negative prognostic factor, or a diagnostic factor.

As used herein, the term “indel” refers to an insertion, a deletion, or both, of one or more nucleotides in a nucleic acid of a cell. In certain instances, an indel includes both an insertion and a deletion of one or more nucleotides, where both the insertion and the deletion are nearby on the nucleic acid. In certain instances, the indel results in a net change in the total number of nucleotides. In certain instances, the indel results in a net change of about 1 to about 50 nucleotides.

As used herein, the terms “variant sequence” or “variant” are used interchangeably and refer to a modified nucleic acid sequence relative to a corresponding “normal” or “wild-type” sequence. In some instances, a variant sequence may be a “short variant sequence” (or “short variant”), i.e., a variant sequence of less than about 50 base pairs in length.

The terms “allele frequency” and “allele fraction” are used interchangeably herein and refer to the fraction of sequence reads corresponding to a particular allele relative to the total number of sequence reads for a genomic locus.

The terms “variant allele frequency” and “variant allele fraction” are used interchangeably herein and refer to the fraction of sequence reads corresponding to a particular variant allele relative to the total number of sequence reads for a genomic locus.

As used herein, the term “library” refers to a collection of nucleic acid molecules. In one instance, the library includes a collection of nucleic acid nucleic acid molecules, e.g., a collection of whole genomic, subgenomic fragments, cDNA, cDNA fragments, RNA, e.g., mRNA, RNA fragments, or a combination thereof. Typically, a nucleic acid molecule is a DNA molecule, e.g., genomic DNA or cDNA. A nucleic acid molecule can be fragmented, e.g., sheared or enzymatically prepared, genomic DNA. Nucleic acid molecules comprise sequence from a subject and can also comprise sequence not derived from the subject, e.g., an adapter sequence, a primer sequence, or other sequences that allow for identification, e.g., “barcode” sequences. In one instance, a portion or all of the library nucleic acid molecules comprises an adapter sequence. The adapter sequence can be located at one or both ends. The adapter sequence can be useful, e.g., for a sequencing method (e.g., an NGS method), for amplification, for reverse transcription, or for cloning into a vector. The library can comprise a collection of nucleic acid molecules, e.g., a target nucleic acid molecule (e.g., a tumor nucleic acid molecule, a reference nucleic acid molecule, or a combination thereof). The nucleic acid molecules of the library can be from a single subject. In instances, a library can comprise nucleic acid molecules from more than one subject (e.g., 2, 3, 4, 5, 6, 7, 8, 9, 10, 20, 30 or more subjects), e.g., two or more libraries from different subjects can be combined to form a library comprising nucleic acid molecules from more than one subject. In one instance, the subject is a human having, or at risk of having, a cancer or tumor.

“Complementary” refers to sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In certain instances, the first region comprises a first portion and the second region comprises a second portion, whereby, when the first and second portions are arranged in an antiparallel fashion, at least about 50%, at least about 75%, at least about 90%, or at least about 95% of the nucleotide residues of the first portion are capable of base pairing with nucleotide residues in the second portion. In other instances, all nucleotide residues of the first portion are capable of base pairing with nucleotide residues in the second portion.

“Likely to” or “increased likelihood,” as used herein, refers to an increased probability that an item, object, thing or person will occur. Thus, in one example, a subject that is likely to respond to treatment has an increased probability of responding to treatment relative to a reference subject or group of subjects.

“Unlikely to” refers to a decreased probability that an event, item, object, thing or person will occur with respect to a reference. Thus, a subject that is unlikely to respond to treatment has a decreased probability of responding to treatment relative to a reference subject or group of subjects.

“Next-generation sequencing” or “NGS” or “NG sequencing” as used herein, refers to any sequencing method that determines the nucleotide sequence of either individual nucleic acid molecules (e.g., in single molecule sequencing) or clonally expanded proxies for individual nucleic acid molecules in a high throughput fashion (e.g., greater than 103, 104, 105 or more molecules are sequenced simultaneously). In one instance, the relative abundance of the nucleic acid species in the library can be estimated by counting the relative number of occurrences of their cognate sequences in the data generated by the sequencing experiment. Next-generation sequencing methods are known in the art, and are described, e.g., in Metzker, M. (2010) Nature Biotechnology Reviews 11:31-46, incorporated herein by reference. Next-generation sequencing can detect a variant present in less than 5% or less than 1% of the nucleic acids in a sample.

“Nucleotide value” as referred herein, represents the identity of the nucleotide(s) occupying or assigned to a nucleotide position. Typical nucleotide values include: missing (e.g., deleted); additional (e.g., an insertion of one or more nucleotides, the identity of which may or may not be included); or present (occupied); A; T; C; or G. Other values can be, e.g., not Y, wherein Y is A, T, G, or C; A or X, wherein X is one or two of T, G, or C; T or X, wherein X is one or two of A, G, or C; G or X, wherein X is one or two of T, A, or C; C or X, wherein X is one or two of T, G, or A; a pyrimidine nucleotide; or a purine nucleotide. A nucleotide value can be a frequency for 1 or more, e.g., 2, 3, or 4, bases (or other value described herein, e.g., missing or additional) at a nucleotide position. E.g., a nucleotide value can comprise a frequency for A, and a frequency for G, at a nucleotide position.

“Or” is used herein to mean, and is used interchangeably with, the term “and/or”, unless context clearly indicates otherwise. The use of the term “and/or” in some places herein does not mean that uses of the term “or” are not interchangeable with the term “and/or” unless the context clearly indicates otherwise.

A “control nucleic acid” or “reference nucleic acid” as used herein, refers to nucleic acid molecules from a control or reference sample. Typically, it is DNA, e.g., genomic DNA, or cDNA derived from RNA, not containing the alteration or variation in the gene or gene product. In certain instances, the reference or control nucleic acid sample is a wild-type or a non-mutated sequence. In certain instances, the reference nucleic acid sample is purified or isolated (e.g., it is removed from its natural state). In other instances, the reference nucleic acid sample is from a blood control, a normal adjacent tissue (NAT), or any other non-cancerous sample from the same or a different subject. In some instances, the reference nucleic acid sample comprises normal DNA mixtures. In some instances, the normal DNA mixture is a process matched control. In some instances, the reference nucleic acid sample has germline variants. In some instances, the reference nucleic acid sample does not have somatic alterations, e.g., serves as a negative control.

As used herein, “target nucleic acid molecule” refers to a nucleic acid molecule that one desires to isolate from the nucleic acid library. In one instance, the target nucleic acid molecules can be a tumor nucleic acid molecule, a reference nucleic acid molecule, or a control nucleic acid molecule, as described herein.

“Tumor nucleic acid molecule,” or other similar term (e.g., a “tumor or cancer-associated nucleic acid molecule”), as used herein refers to a nucleic acid molecule having sequence from a tumor cell. The terms “tumor nucleic acid molecule” and “tumor nucleic acid” may sometimes be used interchangeably herein. In one instance, the tumor nucleic acid molecule includes a subject interval having a sequence (e.g., a nucleotide sequence) that has an alteration (e.g., a mutation) associated with a cancerous phenotype. In other instances, the tumor nucleic acid molecule includes a subject interval having a wild-type sequence (e.g., a wild-type nucleotide sequence). For example, a subject interval from a heterozygous or homozygous wild-type allele present in a cancer cell. A tumor nucleic acid molecule can include a reference nucleic acid molecule. Typically, it is DNA, e.g., genomic DNA, or cDNA derived from RNA, from a sample. In certain instances, the sample is purified or isolated (e.g., it is removed from its natural state). In some instances, the tumor nucleic acid molecule is a cfDNA. In some instances, the tumor nucleic acid molecule is a ctDNA. In some instances, the tumor nucleic acid molecule is DNA from a CTC.

An “isolated” nucleic acid molecule is one which is separated from other nucleic acid molecules which are present in the natural source of the nucleic acid molecule. In certain instances, an “isolated” nucleic acid molecule is free of sequences (such as protein-encoding sequences) which naturally flank the nucleic acid (i.e., sequences located at 5′ and 3′ ends of the nucleic acid) in the genomic DNA of the organism from which the nucleic acid is derived. For example, in various instances, the isolated nucleic acid molecule can contain less than about 5 KB, less than about 4 kB, less than about 3 kB, less than about 2 kB, less than about 1 kB, less than about 0.5 kB or less than about 0.1 kB of nucleotide sequences which naturally flank the nucleic acid molecule in genomic DNA of the cell from which the nucleic acid is derived. Moreover, an “isolated” nucleic acid molecule, such as an RNA molecule or a cDNA molecule, can be substantially free of other cellular material or culture medium, e.g., when produced by recombinant techniques, or substantially free of chemical precursors or other chemicals, e.g., when chemically synthesized.

The section headings used herein are for organizational purposes only and are not to be construed as limiting the subject matter described.

### II. Methods for Determining a Risk Score for Predicting Response to a Selected Treatment for a Disease

Provided herein are methods for comprising determining a risk score for predicting response to a selected treatment for disease in a subject. In some instances, the disease is a cancer. In some instances, the treatment (or treatment regimen) is the FOLFIRINOX or gemcitabine plus paclitaxel regimen.

### A. Risk Score Determination

In some instances, the disclosed methods comprise determining a patient risk score that predicts a response to a selected treatment (e.g., a first-line treatment) for the specified disease based on genomic data. The risk scores provide improved decision-making tools to help healthcare providers choose which of the available treatment options for a given disease (e.g., cancer) is likely to be most effective for a subject.

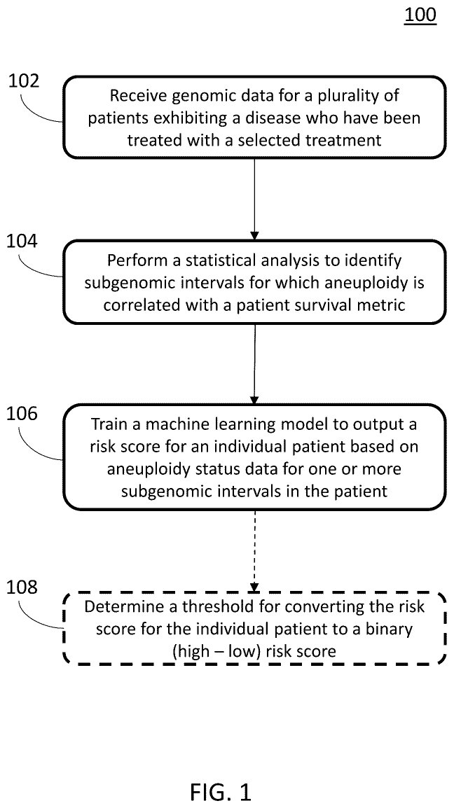

FIG. 1 provides a non-limiting example of a flowchart for a process 100 for determining a risk score that is predictive of a likely response to a selected treatment for a disease in a subject. In some instances, process 100 can be performed, for example, using one or more electronic devices implementing a software platform. In some examples, process 100 is performed using a client-server system, and the blocks of process 100 are divided up in any manner between the server and a client device. In other examples, the blocks of process 100 are divided up between the server and multiple client devices. Thus, while portions of process 100 may be described herein as being performed by particular devices of a client-server system, it will be appreciated that process 100 is not so limited. In other examples, process 100 is performed using only a client device or only multiple client devices. In process 100, some blocks may optionally be combined, the order of some blocks may optionally be changed, and some blocks may optionally be omitted. In some examples, additional steps may be performed in combination with the steps shown for process 100. Accordingly, the operations as illustrated (and as described in greater detail below) are exemplary by nature and, as such, should not be viewed as limiting.

At step 102 in FIG. 1, genomic data for a plurality of patients exhibiting a given disease (e.g., cancer) who have been treated with a selected treatment is received. In some instances, the genomic data may comprise, e.g., genetic mutation data (e.g., point mutation data, insertion data, deletion data, missense mutation data, nonsense mutation data, copy number data), aneuploidy status data, loss of heterozygosity (LOH) data, or any combination thereof, for one or more gene loci and/or one or more subgenomic intervals in each of a plurality of patients exhibiting the disease who have been treated using the selected treatment. In some instances, the genomic data may comprise aneuploidy status data and/or loss of heterozygosity data for one or more subgenomic intervals that comprise chromosome arm-level intervals, e.g., chromosome arm-level aneuploidies and/or chromosome arm-level loss of heterozygosity. In some instances, aneuploidy status data may be determined based on an analysis of genomic data using a method such as that described by Spurr, et al. (2021), “Quantification of Aneuploidy in Targeted Sequencing Data Using ASCETS”, Bioinformatics 2021:1-3. In some instances, loss of heterozygosity (LOH) may be determined based on an analysis of genomic data using a method such as that described by Green, et al. (2010), “A New Method to Detect Loss of Heterozygosity Using Cohort Heterozygosity Comparisons”, BMC Cancer 10:195-203. In some instances, the genomic data may further comprise patient survival data (or other clinical feature data and/or Eastern Cooperative Oncology Group (ECOG) performance data) for the plurality of patients.

In some instances, the genomic data may comprise, or is based on, sequence read data derived from a whole genome sequencing assay. In some instances, the genomic data may comprise, or is based on, sequence read data derived from a targeted sequencing assay. In some instances, the genomic data may comprise, or is based on, sequence read data derived from a comprehensive genomic profiling assay. In some instances, the genomic data may comprise sequence read data for at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, or more than 100 gene loci and/or subgenomic intervals.

At step 104 in FIG. 1, a statistical analysis of the genomic data is performed to identify gene loci and/or subgenomic intervals for which a genetic mutation, ancuploidy status, and/or loss of heterozygosity serves as a biomarker that is correlated with a patient survival metric, e.g., hazard ratio, a progression free survival, an overall survival, a disease-free survival, an objective tumor response rate, a time to tumor progression, a time to treatment failure, a durable complete response, a time to next treatment, or any combination thereof. In some instances, for example, the statistical analysis may comprise a univariable Cox proportional hazards regression analysis, a lasso regression analysis, or a logistic regression analysis.

In some instances, the statistical analysis may further comprise an analysis of clinical feature data for the plurality of patients, e.g., to identify both genetic mutations, ancuploidies, and/or LOH events that, in combination with one or more clinical features, are correlated with the patient survival metric. For example, in some instances, the clinical feature data may comprise patient age, patient sex, patient race, patient clinical history, or any combination thereof.

In some instances, the statistical analysis may further comprise an analysis of Eastern Cooperative Oncology Group (ECOG) performance data for the plurality of patients, e.g., to identify genetic mutations, aneuploidies, LOH events, and/or clinical features that, in combination with the ECOG performance score, are correlated with the patient survival metric.

In some instances, the gene loci and/or subgenomic intervals for which a genetic mutation, aneuploidy status, and/or loss of heterozygosity serves as a biomarker for patient survival may be identified by the statistical analysis (e.g., a univariable Cox proportional hazards regression analysis) as a covariate if the patient survival metric that has a p-value of less than 0.1, less than 0.09, less than 0.08, less than 0.07, less than 0.06, less than 0.05, less than 0.04, less than 0.03, less than 0.02, or less than 0.01. In some instances, the gene loci and/or subgenomic intervals for which a genetic mutation, aneuploidy status, and/or loss of heterozygosity serves as a biomarker for patient survival may be identified by the statistical analysis as a covariate if the patient survival metric that has a p-value of less than 0.05.

In some instances, a covariate set (or feature set) may be determined by performing the statistical analysis in an iterative manner. For example, the genomic data for a cohort of patients who underwent a selected treatment (a treatment cohort) may be split between a training dataset and a test dataset (e.g., using a 90:10, 85:15, or 80:20 split). The statistical analysis (e.g., a univariable Cox proportional hazards regression analysis, forward stepwise regression, backward stepwise regression, bidirectional stepwise regression and lasso regression) may then be performed on the training dataset to identify covariates (gene loci and/or subgenomic intervals for which a genetic mutation, aneuploidy status, and/or loss of heterozygosity is correlated with patient survival) having a p-value of less than, e.g., 0.05. The training dataset (or a specified percentage thereof, e.g., 95%, 90%, 85%, or 80%) may be applied to repeated K-fold cross validation (e.g., 4 repeated 5-fold cross validation, 2 repeated 10-fold cross validation). For each iteration, the statistical analysis is repeated (e.g., covariates for which the p-value is less than 0.05 or other specified threshold) are appended to the covariate set (or feature set). The final set of covariates (or features) may then be determined by keeping only those covariates (or features) that appear in, e.g., greater than 50%, greater than 55%, greater than 60%, greater than 65%, greater than 70%, greater than 75%, greater than 80%, greater than 85%, or greater than 90% of the resampling iterations.

At step 106 in FIG. 1, the genomic data for one or more gene loci and/or subgenomic intervals for which a genetic mutation, aneuploidy status, and/or loss of heterozygosity has been identified (as a result of the statistical analysis performed in step 104 of FIG. 1) to serve as a biomarker that is correlated with the patient survival metric is used (along with the patient survival data) as training data to train a machine learning model, where the machine learning model is configured to receive genomic data for the one or more identified gene loci and/or subgenomic intervals for a subject and output a risk score (e.g., a continuous-valued linear or non-linear risk score) that predicts the likelihood (or probability) that the subject will respond well to the selected treatment.

Any of a variety of machine learning approaches known to those of skill in the art may be used to create the model described at step 106 of FIG. 1. For example, the machine learning method employed may comprise a supervised learning model, an unsupervised learning model, a semi-supervised learning model, a deep learning model, or any combination thereof (see, e.g., Emmert-Streib, et al. (2020), “An Introductory Review of Deep Learning for Prediction Models for Big Data”, Frontiers in Artificial Intelligence, Vol. 3, Article 4; and Mahesh (2020), “Machine Learning Algorithms-A Review”, International Journal of Science and Research 9 (1): 381-386).

In some instances, for example, the machine learning model may comprise an artificial neural network, a deep learning model, a Gaussian process regression model, a multivariable proportional hazards regression model, a decision tree model, a logistical model tree, a random forest model (e.g., a random survival forest model), a conditional inference forest model (e.g., a conditional inference survival forest model), a fuzzy classifier model, a hierarchical clustering model, a k-means clustering model, a fuzzy clustering model, a deep Boltzmann machine learning model, or any combination thereof. In some instances, the machine learning model may comprise a multivariable Cox proportional hazards regression model (e.g., a multivariable Cox model). In some instances, the machine learning model may comprise a conditional inference forest model.

In some instances, the training dataset used to train the machine learning model comprises the final covariate data (or final set of covariate data) identified by the statistical analysis (or by the iterative statistical analysis). The machine learning model may then be trained using any of a variety of training techniques known to those of skill in the art to determine the weighting factors, bias values, threshold values, and/or other computational parameters of the model to ensure that the output of the model (e.g., a risk score) is consistent with the input data in the training data set (and where the choice of training technique and the specific set of trained parameters is typically linked to the choice of machine learning model). Examples of suitable model training techniques that may be used include, but are not limited to, gradient descent methods, backward propagation methods, iterative self-training methods, and the like. In some instances, two or more training data sets (e.g., comprising genomic data for two or more patient cohorts) may be used to train the model.

Multivariable Cox model: In one non-limiting example, the training dataset comprising the final covariate data (or final set of covariate data) may be used to train a multivariable Cox proportional hazards regression model (also referred to herein as a “multivariable Cox model”) for outputting a patient risk score based on genomic data, or on both genomic data and clinical data. A multivariable Cox proportional hazards regression model is an example of a survival model that relates the quantity of time that passes until a specified event occurs (e.g., patient death following initiation of a given disease treatment, as expressed by a hazard function) to one or more covariates that may be associated with that quantity of time (see, e.g., Bradburn, et al. (2003), “Survival Analysis Part II: Multivariate Data Analysis—An Introduction to Concepts and Methods”, British Journal of Cancer 89, 431-436). In a proportional hazards model, a specified increase in a given covariate results in a proportional scaling of the hazard. The multivariable Cox proportional hazards regression model extends survival analysis methods to assess simultaneously the effect of several risk factors on survival time.

The multivariable Cox model is based on the hazard function, h (t), which describes the risk of dying at time t under a specified set of conditions (e.g., following treatment of a given patient cohort by a specified disease therapy), and is given by the equation:

$$h ( t ) = {h}_{0} ( t ) * exp ( {b}_{1} {x}_{1} + {b}_{2} {x}_{2} + … + {b}_{p} {x}_{p} )$$

where t is the survival time, h (t) is the hazard function determined by a set of p covariates (x₁, x₂, . . . , xₚ), the coefficients (b₁, b₂, . . . , bₚ) describe the relative impact of the corresponding covariates, and ho is the baseline hazard. The Cox model can thus be viewed as a multiple linear regression of the logarithm of h(t) on the variables xᵢ, with the baseline hazard corresponding to an ‘intercept’ term that varies with time. The quantities exp (bᵢ) are called hazard ratios (HR). A value of bᵢ greater than zero (or a hazard ratio of greater than one) indicates that as the value of the corresponding covariate increases, the event hazard increases and thus the length of survival decreases. A value of b; equal to zero (or a hazard ratio equal to one) indicates that the corresponding covariate has no effect on hazard or length of survival. A value of b; less than zero (or a hazard ratio of less than one) indicates that as the value of the corresponding covariate increases, the event hazard decreases and thus the length of survival increases.

The Cox model is trained on the training dataset (e.g., fit to the training data) to determine the values of the coefficients (b₁, b₂, . . . , bₚ) that provide the most accurate correlation between the set of covariates and patient survival times. For example, in some instances, a stepwise regression procedure (e.g., a bidirectional stepwise regression procedure) may be used to train the multivariable Cox model. Stepwise regression is a method of fitting regression models in which the choice of predictive variables is carried out in an automated fashion. At each step, a variable is considered for addition to, or subtraction from, the set of predictive variables included in the model based on a specified criterion, e.g., a forward, backward, or combined sequence of F-tests or t-tests. Examples of the approaches used for stepwise regression are:

Forward selection, in which-starting with no candidate variables included in the model—candidate variables are tested for inclusion using a specified model fit criterion, and added to the model if their inclusion gives a statistically significant improvement of the model fit; the process is repeated until there are no remaining candidate variables for which inclusion provides a statistically significant improvement of the model.

Backward elimination, in which-starting with all candidate variables included in the model—deletion of candidate variables is tested using a specified model fit criterion, and the candidate variables whose loss gives the most statistically insignificant deterioration of the model fit are deleted; the process is repeated until no additional variables can be deleted without incurring a statistically significant loss of fit.

Bidirectional elimination (a combination of forward selection and backward elimination), in which candidate variables are tested at each step using a specified model fit criterion for inclusion or exclusion.

In some instances, other criteria may be used to select a best fit model from a set of candidate models based on different combinations of predictive variables. Examples of such model selection criteria include, but are not limited to, the Akaike information criterion, the Bayesian information criterion, a Calinski Harabasz score, false discovery rate, and the like.

Random forest models and conditional inference forest models: In another non-limiting example, the training dataset comprising the final covariate data (or final set of covariate data) may be used to train a random forest model (e.g., a random survival forest model) or a conditional inference forest (CIF) model (e.g., a conditional inference survival forest model) to output a patient risk score based on genomic data. Survival trees and forests are non-parametric models that may be used for time-to-event analysis (see, e.g., Nasejje, et al. (2017), “A comparison of the conditional inference survival forest model to random survival forests based on a simulation study as well as on two applications with time-to-event data”, BMC Medical Research Methodology 17:115). A survival tree is based on the idea of partitioning the covariate space recursively to form groups of subjects who are similar according to the time-to-event outcome.

A common approach for building a survival tree is to use a binary split of a parent node (e.g., a group of patients) based on a single covariate (or predictor of patient survival) to obtain two daughter nodes that differ according to an impurity metric or split rule (Nasejje, et al. (2017), ibid.), with the goal of identifying factors that are predictive of the time-to-event outcome. For a categorical covariate X (e.g., a covariate that can that can take on one of a limited number of possible values), a split is defined as X≤c where c is a constant (i.e., where the data for X≤c belongs to one daughter node, and data for X>c belongs to the other daughter node). For a categorical covariate X with multiple split-points, a potential split is defined as X ∈ {c₁, . . . , cₖ/where c₁, . . . , cₖ are potential split values. The choice of impurity metric (or split-rule) used for building the survival tree has a significant impact on the prediction accuracy of the model. Examples of split-rules (or impurity metrics) used for the analysis of time-to-event data include, but are not limited to, (i) the log-rank split rule (where the best split of a parent node into two daughter nodes on a covariate X at a given split point is the one that gives the largest log-rank statistic (a statistic that compares estimates of the hazard functions of two daughter nodes at each observed event time)), (ii) the log-rank score split-rule (a modification of the log-rank split rule where the best split is the one that gives the maximum absolute value of the log-rank score, S (X, c), over X and c (see, e.g., Waititu, et al. (2021), “Analysis of Balanced Random Survival Forest Using Different Splitting Rules: Application on Child Mortality”, International Journal of Statistics and Applications 11 (2): 37-49)), and (iii) the gradient-based Brier score split-rule (a frequently-used scalar summary of correctness for probability predictions for binary events; see, e.g., Waititu, et al. (2021), ibid.). A random survival forest model combines the output of multiple (randomly-created) survival trees to generate the final output.

Conditional inference survival forests are a variation on random survival forest (RSF) models that are known to correct bias in RSF models that results from favoring those covariates that have many split-points (Nasejje, et al. (2017), ibid.). CIF models overcome this bias by separating the procedure for identifying the best covariate to split on from that of the best split point search for the selected covariate. Conditional inference trees use a significance test to select input variables rather than selecting the variable that maximizes an information measure.

A non-limiting example of the basic approach for building a conditional inference tree (Nasejje, et al. (2017), ibid.) comprises the following three steps. Step 1: for case weights, w, select the covariate Xⱼ* with the strongest correlation with survival time T. Step 2: select a subset A* of the values of Xⱼ* to create two disjoint subsets, A* and Xⱼ*/A*, and evaluate the weights wₐ and we for A* and Xⱼ*/A*, respectively. Step 3: recursively repeat steps 1 and 2 with modified case weights wₐ and wβ, respectively. For time-to-event data, the optimal split-variable in step 1 is obtained by testing the association of all the covariates to the time-to-event outcome using, e.g., an appropriate linear rank test. The covariate with the strongest association to the time-to-event outcome based on testing all possible permutations is selected for splitting. Using the distribution of the rank statistic resulting from performing, e.g., a log-rank score test, p-values are evaluated and the covariate with minimum p-value is selected as having the strongest association to the outcome. A standard binary split is done in the second step. A single conditional inference tree (e.g., a single conditional inference survival tree) is generally unstable with respect to prediction accuracy, thus a forest of (randomly-generated) conditional inference trees may be combined into a conditional inference forest (CIF) model (e.g., a conditional inference forest survival model).

Returning to FIG. 1, at step 108, a threshold for converting a continuous-valued risk score output by the trained machine learning model for the subject to a binary (e.g., high-low) risk score may optionally be determined. For example, in some instances the mean, median, or mode of the risk score output by the trained machine learning model for the cohort of patients whose data was used to train the model may be used as a cut-off threshold for discriminating between high risk and low risk patients where a low risk score indicates that the subject is likely to survive longer than a patient with a high risk score if treated with the selected treatment. In some instances, a cut-off threshold may be determined according to the value of a linear risk score output by the trained machine learning model that maximizes a log rank statistic for the risk scores for the patient cohort used to generate the training data.

In some instances, the methods illustrated by the flowchart in FIG. 1 may be applied to identifying biomarkers and/or developing treatment decision-making tools for any of a variety of diseases (e.g., cancers) for which patient survival data and patient genomic data is available. In some instances, for example, the disclosed methods may be applied to patients diagnosed with metastatic pancreatic cancer.

As will be discussed in the examples below, the disclosed methods may be used to identify biomarkers and develop treatment decision-making tools for metastatic pancreatic cancer patients where the treatment options comprise FOLFIRINOX or gemcitabine plus albumin-bound paclitaxel. In some instances, for example, where the selected treatment comprises FOLFIRINOX, one or more subgenomic intervals for which aneuploidy or LOH is correlated with the patient survival metric may comprise Chr3q, Chr4p, Chr5p, Chr5q, Chr7q, Chr11p, Chr12p, Chr12q, Chr15q, Chr16p, Chr17p, Chr19p, Chr19q, Chr20p, Chr22q, or any combination thereof. In some instances, for example, where the selected treatment comprises FOLFIRINOX, one or more subgenomic intervals for which aneuploidy or LOH is correlated with the patient survival metric may comprise Chr7q, Chr15q, or any combination thereof. In some instances, for example, where the selected treatment comprises gemcitabine plus albumin-bound paclitaxel, one or more subgenomic intervals for which aneuploidy or LOH is correlated with the patient survival metric may comprise Chr1p, Chr1q, Chr3p, Chr6p, Chr6q, Chr7p, Chr7q, Chr8q, Chr9p, Chr9q, Chr14q, Chr15q, Chr16p, Chr17p, Chr17q, Chr18q, Chr19p, Chr20p, Chr21p, Chr21q, Chr22q, or any combination thereof. In some instances, for example, where the selected treatment comprises gemcitabine plus albumin-bound paclitaxel, one or more subgenomic intervals for which aneuploidy or LOH is correlated with the patient survival metric may comprise Chr3p, Chr6p, Chr8q, Chr9q, Chr18q, Chr20p, Chr21p, Chr21q, Chr22q, or any combination thereof.

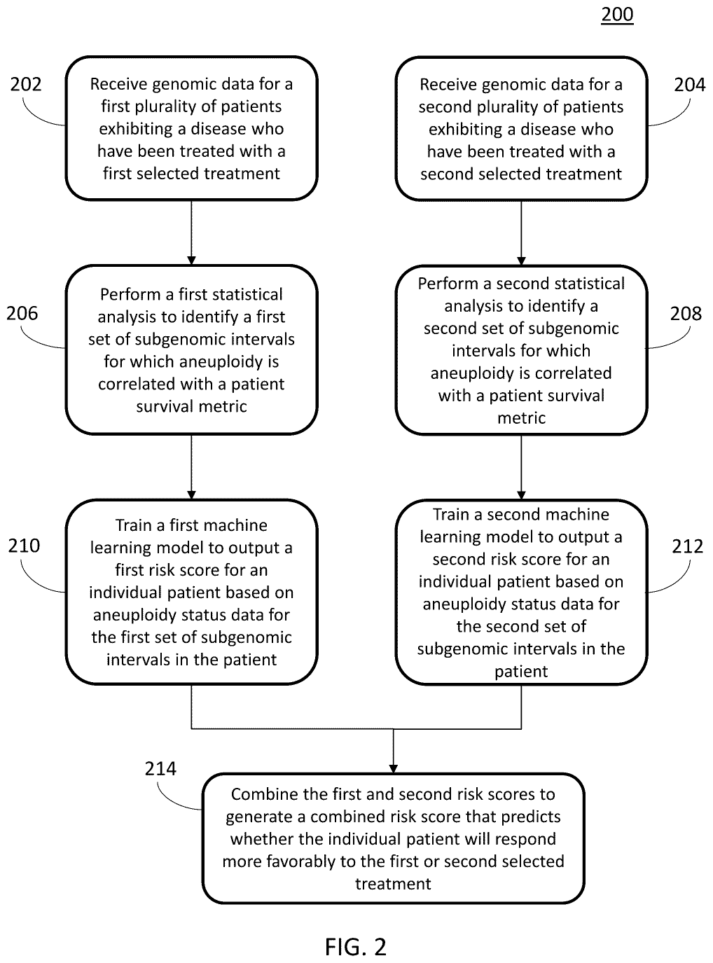

FIG. 2 provides a non-limiting example of a flowchart for a process 200 for determining a combined risk score that predicts whether a subject will respond more favorably to a first selected treatment or to a second selected treatment.

At step 202 in FIG. 2, genomic data for a first plurality of patients exhibiting a given disease (e.g., cancer) who have been treated with a first selected treatment is received.

At step 204 in FIG. 2, genomic data for a second plurality of patients exhibiting the given disease (e.g., cancer) who have been treated with a second selected treatment is received.

As indicated above for the description of FIG. 1, in some instances the genomic data for the first and/or second plurality of patients may comprise, e.g., genetic mutation data (e.g., point mutation data, insertion data, deletion data, missense mutation data, nonsense mutation data, copy number data), aneuploidy status data, loss of heterozygosity (LOH) data, or any combination thereof, for one or more gene loci and/or one or more subgenomic intervals in each of the plurality of patients exhibiting the disease who have been treated using the selected treatment. In some instances, the genomic data may comprise aneuploidy status data and/or loss of heterozygosity data for one or more subgenomic intervals that comprise chromosome arm-level intervals, e.g., chromosome arm-level aneuploidies and/or chromosome arm-level loss of heterozygosity. In some instances, the genomic data may further comprise patient survival data (or other clinical feature data and/or Eastern Cooperative Oncology Group (ECOG) performance data) for each of the pluralities of patients.

At step 206 in FIG. 2, a first statistical analysis of the genomic data for the first plurality of patients is performed to identify gene loci and/or subgenomic intervals for which a genetic mutation, aneuploidy status, and/or loss of heterozygosity serves as a biomarker that is correlated with a patient survival metric, e.g., hazard ratio, a progression free survival, or any combination thereof. As indicated above, in some instances aneuploidy status data may be determined based on an analysis of genomic data using a method such as that described by Spurr, et al. (2020), “Quantification of Aneuploidy in Targeted Sequencing Data Using ASCETS”, Bioinformatics 2020:1-3. In some instances, loss of heterozygosity (LOH) may be determined based on an analysis of genomic data using a method such as that described by Green, et al. (2010), “A New Method to Detect Loss of Heterozygosity Using Cohort Heterozygosity Comparisons”, BMC Cancer 10:195-203.

At step 208 in FIG. 2, a second statistical analysis of the genomic data for the second plurality of patients is performed to identify gene loci and/or subgenomic intervals for which a genetic mutation, aneuploidy status, and/or loss of heterozygosity serves as a biomarker that is correlated with a patient survival metric, e.g., hazard ratio, a progression free survival, or any combination thereof.

In some instances, the first and/or second statistical analysis may comprise a univariable Cox proportional hazards regression analysis. In some instances, the first and/or second statistical analysis may further comprise an analysis of clinical feature data for the first and/or second plurality of patients, respectively (e.g., to identify both genetic mutations, aneuploidies, and/or LOH events that, in combination with one or more clinical features, are correlated with the patient survival metric). For example, in some instances, the clinical feature data may comprise patient age, patient sex, patient race, patient clinical history, or any combination thereof.

In some instances, the first and/or second statistical analysis may further comprise an analysis of Eastern Cooperative Oncology Group (ECOG) performance data for the first and/or second plurality of patients, e.g., to identify genetic mutations, aneuploidies, LOH events, and/or clinical features that, in combination with the ECOG performance score, are correlated with the patient survival metric.

In some instances, the gene loci and/or subgenomic intervals for which a genetic mutation, aneuploidy status, and/or loss of heterozygosity serves as a biomarker for patient survival may be identified by the first and/or second statistical analysis (e.g., a univariable Cox proportional hazards regression analysis) as a covariate if the patient survival metric that has a p-value of less than 0.1, less than 0.09, less than 0.08, less than 0.07, less than 0.06, less than 0.05, less than 0.04, less than 0.03, less than 0.02, or less than 0.01. In some instances, the gene loci and/or subgenomic intervals for which a genetic mutation, aneuploidy status, and/or loss of heterozygosity serves as a biomarker for patient survival may be identified by the statistical analysis as a covariate if the patient survival metric that has a p-value of less than 0.05.

In some instances, a covariate set (or feature set) for the first and/or second plurality of patients may be determined by performing the first and/or second statistical analysis in an iterative manner as described above for step 104 of FIG. 1.

At step 210 of FIG. 2, the genomic data for one or more gene loci and/or subgenomic intervals for which a genetic mutation, aneuploidy status, and/or loss of heterozygosity has been identified to serve as a biomarker that is correlated with the patient survival metric in the first plurality of patients is used (along with the patient survival data) as training data to train a first machine learning model, where the first machine learning model is configured to receive genomic data for the one or more identified gene loci and/or subgenomic intervals for a subject and output a first risk score (e.g., a binary risk score, or a continuous-valued linear or non-linear risk score) that predicts the likelihood (or probability) that the patient will respond well to the first selected treatment.

At step 212 of FIG. 2, the genomic data for one or more gene loci and/or subgenomic intervals for which a genetic mutation, aneuploidy status, and/or loss of heterozygosity has been identified to serve as a biomarker that is correlated with the patient survival metric in the second plurality of patients is used (along with the patient survival data) as training data to train a second machine learning model, where the second machine learning model is configured to receive genomic data for the one or more identified gene loci and/or subgenomic intervals for a subject and output a second risk score (e.g., a binary risk score, or a continuous-valued linear or non-linear risk score) that predicts the likelihood (or probability) that the patient will respond well to the second selected treatment.

As indicated above for the description of FIG. 1, any of a variety of machine learning approaches known to those of skill in the art may be used to create the first and/or second machine learning models. For example, the machine learning method employed for the first and/or second machine learning model may comprise a supervised learning model, an unsupervised learning model, a semi-supervised learning model, a deep learning model, or any combination thereof. In some instances, for example, the first and/or second machine learning model may comprise an artificial neural network, a deep learning model, a Gaussian process regression model, a multivariable proportional hazards regression model, a decision tree model, a logistical model tree, a random forest model (e.g., a random survival forest model), a conditional inference forest model (e.g., a conditional inference survival forest model), a fuzzy classifier model, a hierarchical clustering model, a k-means clustering model, a fuzzy clustering model, a deep Boltzmann machine learning model, or any combination thereof. In some instances, the first and/or second machine learning model may comprise a multivariable Cox proportional hazards regression model (e.g., a multivariable Cox model). In some instances, the first and/or second machine learning model may comprise a conditional inference forest model.

In some instances, the training dataset used to train the first and/or second machine learning model comprises the final covariate data (or final set of covariate data) identified by the first and/or second statistical analysis (or by the iterative statistical analysis performed on the genomic data for the first and/or second plurality of patients). The first and/or second machine learning model may then be trained using any of a variety of training techniques known to those of skill in the art to determine the weighting factors, bias values, threshold values, and/or other computational parameters of the model to ensure that the output of the model (e.g., a risk score) is consistent with the input data in the training data set (and where the choice of training technique and the specific set of trained parameters is typically linked to the choice of machine learning model). Examples of suitable model training techniques that may be used include, but are not limited to, gradient descent methods, backward propagation methods, iterative self-training methods, and the like. In some instances, two or more training data sets (e.g., comprising genomic data for two or more patient cohorts) may be used to train the first and/or second model.

At step 214 in FIG. 2, the first and second risk scores may be combined to generate a combined risk score that predicts whether the subject will respond more favorably to the first or second selected treatment. Optionally, e.g., in cases where the first and second risk scores are continuous-valued, the first and second risk scores may be subjected to first and second cut-off thresholds respectively to convert them to binary (e.g., high-low) first and second risk scores. Cut-off thresholds may be determined as described above for step 108 in FIG. 1. In some instances, determining a combined risk score may comprise adding the first and second risk scores, subtracting the first and second risk scores, multiplying the first and second risk scores, dividing the first and second risk scores, multiplying the first and second risk scores by corresponding first and second weighting factors followed by adding, subtracting, or dividing the weighted first and second risk scores, or taking an average, mean, or median of the first and second risk factors. In some instances, both the first and second risk scores may be utilized to identify the best treatment for the subject. That is, the first and second risk scores may be used as separate covariates in a multivariable Cox model to estimate treatment effect (e.g., to determine a hazard ratio for FOLF vs. G+P treatment). In some instances, interaction terms between treatment and risk score would be included. In some instances, clinical covariates could also be included to account for clinical imbalances between the cohorts. In some instances, a propensity score method could be used to balance clinical bias.

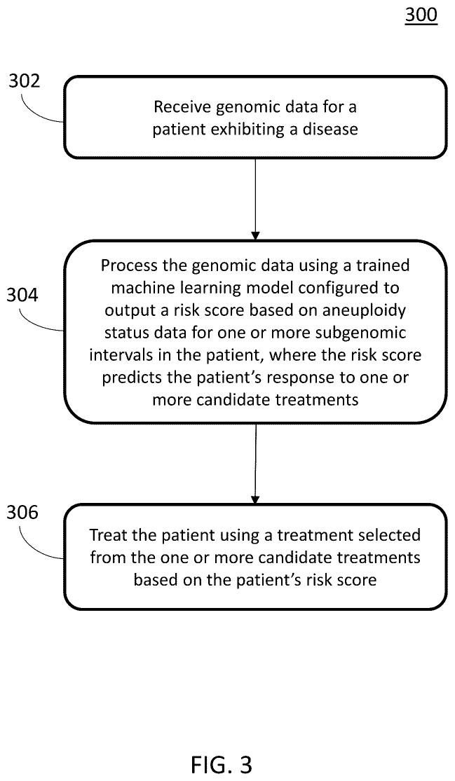

FIG. 3 provides a non-limiting example of a flowchart for a process 300 for selecting a treatment and treating a subject according to the methods described herein. At step 302 in FIG. 3, genomic data for a patient exhibiting a disease (e.g., cancer) is received, where the genomic data may comprise, e.g., genetic mutation data (e.g., point mutation data, insertion data, deletion data, missense mutation data, nonsense mutation data, copy number data), aneuploidy status data, loss of heterozygosity (LOH) data, or any combination thereof, for one or more gene loci and/or one or more subgenomic intervals in each of a plurality of patients exhibiting the disease who have been treated using the selected treatment.

As indicated above, in some instances aneuploidy status data may be determined based on an analysis of genomic data using a method such as that described by Spurr, et al. (2020), “Quantification of Aneuploidy in Targeted Sequencing Data Using ASCETS”, Bioinformatics 2020:1-3. In some instances, loss of heterozygosity (LoH) may be determined based on an analysis of genomic data using a method such as that described by Green, et al. (2010), “A New Method to Detect Loss of Heterozygosity Using Cohort Heterozygosity Comparisons”, BMC Cancer 10:195-203.

In some instances, the genomic data may comprise aneuploidy status data and/or loss of heterozygosity data for one or more subgenomic intervals that comprise chromosome arm-level intervals, e.g., chromosome arm-level aneuploidies and/or chromosome arm-level loss of heterozygosity. In some instances, the genomic data may further comprise patient survival data (or other clinical feature data and/or Eastern Cooperative Oncology Group (ECOG) performance data) for the plurality of patients.

At step 304 in FIG. 3, the genomic data is processed using a trained machine learning model configured to output a risk score based on, e.g., aneuploidy status and/or loss of heterozygosity (LOH) data for one or more subgenomic intervals in the patient, where the risk score predicts the patient's response to one or more candidate treatments for the disease.

At step 306 in FIG. 3, the patient may be treated using a treatment selected by a healthcare provider from the one or more candidate treatments based on the patient's risk score.

### B. Samples

The disclosed methods and systems may be used with any of a variety of samples (also referred to herein as specimens) comprising nucleic acids (e.g., DNA or RNA) that are collected from a subject. Examples of a sample include, but are not limited to, a tumor sample, a tissue sample, a biopsy sample (e.g., a tissue biopsy, a liquid biopsy, or both), a blood sample (e.g., a peripheral whole blood sample), a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces (or stool) sample, or other body fluid, secretion, and/or excretion sample (or cell sample derived therefrom). In certain instances, the sample may be frozen sample or a formalin-fixed paraffin-embedded (FFPE) sample.

In some instances, the sample may be collected by tissue resection (e.g., surgical resection), needle biopsy, bone marrow biopsy, bone marrow aspiration, skin biopsy, endoscopic biopsy, fine needle aspiration, oral swab, nasal swab, vaginal swab or a cytology smear, scrapings, washings or lavages (such as a ductal lavages or bronchoalveolar lavages), etc.

In some instances, the sample is a liquid biopsy sample, and may comprise, e.g., whole blood, blood plasma, blood serum, urine, stool, sputum, saliva, or cerebrospinal fluid. In some instances, the sample may be a liquid biopsy sample and may comprise circulating tumor cells (CTCs). In some instances, the sample may be a liquid biopsy sample and may comprise mRNA, DNA, cell-free DNA (cfDNA), circulating tumor DNA (ctDNA), cell-free RNA from a cancer, or any combination thereof.

In some instances, the sample may comprise one or more premalignant or malignant cells. Premalignant, as used herein, refers to a cell or tissue that is not yet malignant but is poised to become malignant. In certain instances, the sample may be acquired from a solid tumor, a soft tissue tumor, or a metastatic lesion. In certain instances, the sample may be acquired from a hematologic malignancy or pre-malignancy. In other instances, the sample may comprise a tissue or cells from a surgical margin. In certain instances, the sample may comprise tumor-infiltrating lymphocytes. In some instances, the sample may comprise one or more non-malignant cells. In some instances, the sample may be, or is part of, a primary tumor or a metastasis (e.g., a metastasis biopsy sample). In some instances, the sample may be obtained from a site (e.g., a tumor site) with the highest percentage of tumor (e.g., tumor cells) as compared to adjacent sites (e.g., sites adjacent to the tumor). In some instances, the sample may be obtained from a site (e.g., a tumor site) with the largest tumor focus (e.g., the largest number of tumor cells as visualized under a microscope) as compared to adjacent sites (e.g., sites adjacent to the tumor).

In some instances, the disclosed methods may further comprise analyzing a primary control (e.g., a normal tissue sample). In some instances, the disclosed methods may further comprise determining if a primary control is available and, if so, isolating a control nucleic acid (e.g., DNA) from said primary control. In some instances, the sample may comprise any normal control (e.g., a normal adjacent tissue (NAT)) if no primary control is available. In some instances, the sample may be or may comprise histologically normal tissue. In some instances, the method includes evaluating a sample, e.g., a histologically normal sample (e.g., from a surgical tissue margin) using the methods described herein. In some instances, the disclosed methods may further comprise acquiring a sub-sample enriched for non-tumor cells, e.g., by macro-dissecting non-tumor tissue from said NAT in a sample not accompanied by a primary control. In some instances, the disclosed methods may further comprise determining that no primary control and no NAT is available, and marking said sample for analysis without a matched control.

In some instances, samples obtained from histologically normal tissues (e.g., otherwise histologically normal surgical tissue margins) may still comprise a genetic alteration such as a variant sequence as described herein. The methods may thus further comprise re-classifying a sample based on the presence of the detected genetic alteration. In some instances, multiple samples (e.g., from different subjects) are processed simultaneously.

The disclosed methods and systems may be applied to the analysis of nucleic acids extracted from any of variety of tissue samples (or disease states thereof), e.g., solid tissue samples, soft tissue samples, metastatic lesions, or liquid biopsy samples. Examples of tissues include, but are not limited to, connective tissue, muscle tissue, nervous tissue, epithelial tissue, and blood. Tissue samples may be collected from any of the organs within an animal or human body. Examples of human organs include, but are not limited to, the brain, heart, lungs, liver, kidneys, pancreas, spleen, thyroid, mammary glands, uterus, prostate, large intestine, small intestine, bladder, bone, skin, etc.

In some instances, the nucleic acids extracted from the sample may comprise deoxyribonucleic acid (DNA) molecules. Examples of DNA that may be suitable for analysis by the disclosed methods include, but are not limited to, genomic DNA or fragments thereof, mitochondrial DNA or fragments thereof, cell-free DNA (cfDNA), and circulating tumor DNA (ctDNA). Cell-free DNA (cfDNA) is comprised of fragments of DNA that are released from normal and/or cancerous cells during apoptosis and necrosis, and circulate in the blood stream and/or accumulate in other bodily fluids. Circulating tumor DNA (ctDNA) is comprised of fragments of DNA that are released from cancerous cells and tumors that circulate in the blood stream and/or accumulate in other bodily fluids.

In some instances, DNA is extracted from nucleated cells from the sample. In some instances, a sample may have a low nucleated cellularity, e.g., when the sample is comprised mainly of erythrocytes, lesional cells that contain excessive cytoplasm, or tissue with fibrosis. In some instances, a sample with low nucleated cellularity may require more, e.g., greater, tissue volume for DNA extraction.

In some instances, the nucleic acids extracted from the sample may comprise ribonucleic acid (RNA) molecules. Examples of RNA that may be suitable for analysis by the disclosed methods include, but are not limited to, total cellular RNA, total cellular RNA after depletion of certain abundant RNA sequences (e.g., ribosomal RNAs), cell-free RNA (cfRNA), messenger RNA (mRNA) or fragments thereof, the poly (A)-tailed mRNA fraction of the total RNA, ribosomal RNA (rRNA) or fragments thereof, transfer RNA (tRNA) or fragments thereof, and mitochondrial RNA or fragments thereof. In some instances, RNA may be extracted from the sample and converted to complementary DNA (cDNA) using, e.g., a reverse transcription reaction. In some instances, the cDNA is produced by random-primed cDNA synthesis methods. In other instances, the cDNA synthesis is initiated at the poly (A) tail of mature mRNAs by priming with oligo (dT)-containing oligonucleotides. Methods for depletion, poly (A) enrichment, and cDNA synthesis are well known to those of skill in the art.

In some instances, the sample may comprise a tumor content (e.g., comprising tumor cells or tumor cell nuclei), or a non-tumor content (e.g., immune cells, fibroblasts, and other non-tumor cells). In some instances, the tumor content of the sample may constitute a sample metric. In some instances, the sample may comprise a tumor content of at least 5-50%, 10-40%, 15-25%, or 20-30% tumor cell nuclei. In some instances, the sample may comprise a tumor content of at least 5%, at least 10%, at least 20%, at least 30%, at least 40%, or at least 50% tumor cell nuclei. In some instances, the percent tumor cell nuclei (e.g., sample fraction) is determined (e.g., calculated) by dividing the number of tumor cells in the sample by the total number of all cells within the sample that have nuclei. In some instances, for example when the sample is a liver sample comprising hepatocytes, a different tumor content calculation may be required due to the presence of hepatocytes having nuclei with twice, or more than twice, the DNA content of other, e.g., non-hepatocyte, somatic cell nuclei. In some instances, the sensitivity of detection of a genetic alteration, e.g., a variant sequence, or a determination of, e.g., microsatellite instability, may depend on the tumor content of the sample. For example, a sample having a lower tumor content can result in lower sensitivity of detection for a given size sample.

In some instances, as noted above, the sample comprises nucleic acid (e.g., DNA, RNA (or a cDNA derived from the RNA), or both), e.g., from a tumor or from normal tissue. In certain instances, the sample may further comprise a non-nucleic acid component, e.g., cells, protein, carbohydrate, or lipid, e.g., from the tumor or normal tissue.

### C. Subjects

In some instances, the sample is obtained (e.g., collected) from a subject with a condition or disease (e.g., a hyperproliferative disease or a non-cancer indication) or suspected of having the condition or disease. In some instances, the hyperproliferative disease is a cancer. In some instances, the cancer is a solid tumor or a metastatic form thereof. In some instances, the cancer is a hematological cancer, e.g. a leukemia or lymphoma.

In some instances, the subject has a cancer or is at risk of having a cancer. For example, in some instances, the subject has a genetic predisposition to a cancer (e.g., having a genetic mutation that increases his or her baseline risk for developing a cancer). In some instances, the subject has been exposed to an environmental perturbation (e.g., radiation or a chemical) that increases his or her risk for developing a cancer. In some instances, the subject is in need of being monitored for development of a cancer. In some instances, the subject is in need of being monitored for cancer progression or regression, e.g., after being treated with an anti-cancer therapy (or anti-cancer treatment). In some instances, the subject is in need of being monitored for relapse of cancer. In some instances, the subject is in need of being monitored for minimum residual disease (MRD). In some instances, the subject has been, or is being treated, for cancer. In some instances, the subject has not been treated with an anti-cancer therapy (or anti-cancer treatment).

In some instances, the subject is being treated, or has been previously treated, with one or more targeted therapies. In some instances, e.g., for a subject who has been previously treated with a targeted therapy, a post-targeted therapy sample (e.g., specimen) is obtained (e.g., collected). In some instances, the post-targeted therapy sample is a sample obtained after the completion of the targeted therapy.

In some instances, the subject has not been previously treated with a targeted therapy. In some instances, e.g., for a subject who has not been previously treated with a targeted therapy, the sample comprises a resection, e.g., an original resection, or a resection following recurrence (e.g., following a disease recurrence post-therapy).

In some instances, the subject is a human. In some instances, the subject is a non-human mammal.

### D. Cancers

In some instances, the sample is acquired from a subject having a cancer. Exemplary cancers include, but are not limited to, B cell cancer (e.g., multiple myeloma), melanomas, breast cancer, lung cancer (such as non-small cell lung carcinoma or NSCLC), bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, adenocarcinomas, inflammatory myofibroblastic tumors, gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pincaloma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancers, essential thrombocythemia, agnogenic myeloid metaplasia, hypercosinophilic syndrome, systemic mastocytosis, familiar hypereosinophilia, chronic cosinophilic leukemia, neuroendocrine cancers, carcinoid tumors, and the like. In some instances, the cancer is a pancreatic cancer. In some instances, the pancreatic cancer is a metastatic pancreatic cancer.

In some instances, the cancer is a hematologic malignancy (or premaligancy). As used herein, a hematologic malignancy refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMOL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sézary syndrome, Waldenström macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm.

### E. Nucleic Acid Extraction and Processing

DNA or RNA may be extracted from tissue samples, biopsy samples, blood samples, or other bodily fluid samples using any of a variety of techniques known to those of skill in the art (see, e.g., Example 1 of International Patent Application Publication No. WO 2012/092426; Tan, et al. (2009), “DNA, RNA, and Protein Extraction: The Past and The Present”, J. Biomed. Biotech. 2009:574398; the technical literature for the Maxwell® 16 LEV Blood DNA Kit (Promega Corporation, Madison, WI); and the Maxwell 16 Buccal Swab LEV DNA Purification Kit Technical Manual (Promega Literature #TM333, Jan. 1, 2011, Promega Corporation, Madison, WI)). Protocols for RNA isolation are disclosed in, e.g., the Maxwell® 16 Total RNA Purification Kit Technical Bulletin (Promega Literature #TB351, August 2009, Promega Corporation, Madison, WI).

A typical DNA extraction procedure, for example, comprises (i) collection of the fluid sample, cell sample, or tissue sample from which DNA is to be extracted, (ii) disruption of cell membranes (i.e., cell lysis), if necessary, to release DNA and other cytoplasmic components, (iii) treatment of the fluid sample or lysed sample with a concentrated salt solution to precipitate proteins, lipids, and RNA, followed by centrifugation to separate out the precipitated proteins, lipids, and RNA, and (iv) purification of DNA from the supernatant to remove detergents, proteins, salts, or other reagents used during the cell membrane lysis step.

Disruption of cell membranes may be performed using a variety of mechanical shear (e.g., by passing through a French press or fine needle) or ultrasonic disruption techniques. The cell lysis step often comprises the use of detergents and surfactants to solubilize lipids the cellular and nuclear membranes. In some instances, the lysis step may further comprise use of proteases to break down protein, and/or the use of an RNase for digestion of RNA in the sample.

Examples of suitable techniques for DNA purification include, but are not limited to, (i) precipitation in ice-cold ethanol or isopropanol, followed by centrifugation (precipitation of DNA may be enhanced by increasing ionic strength, e.g., by addition of sodium acetate), (ii) phenol-chloroform extraction, followed by centrifugation to separate the aqueous phase containing the nucleic acid from the organic phase containing denatured protein, and (iii) solid phase chromatography where the nucleic acids adsorb to the solid phase (e.g., silica or other) depending on the pH and salt concentration of the buffer.

In some instances, cellular and histone proteins bound to the DNA may be removed either by adding a protease or by having precipitated the proteins with sodium or ammonium acetate, or through extraction with a phenol-chloroform mixture prior to a DNA precipitation step.

In some instances, DNA may be extracted using any of a variety of suitable commercial DNA extraction and purification kits. Examples include, but are not limited to, the QIAamp (for isolation of genomic DNA from human samples) and DNAcasy (for isolation of genomic DNA from animal or plant samples) kits from Qiagen (Germantown, MD) or the Maxwell® and ReliaPrep™ series of kits from Promega (Madison, WI).

As noted above, in some instances the sample may comprise a formalin-fixed (also known as formaldehyde-fixed, or paraformaldehyde-fixed), paraffin-embedded (FFPE) tissue preparation. For example, the FFPE sample may be a tissue sample embedded in a matrix, e.g., an FFPE block. Methods to isolate nucleic acids (e.g., DNA) from formaldehyde-or paraformaldehyde-fixed, paraffin-embedded (FFPE) tissues are disclosed in, e.g., Cronin, et al., (2004) Am J Pathol. 164 (1): 35-42; Masuda, et al., (1999) Nucleic Acids Res. 27 (22): 4436-4443; Specht, et al., (2001) Am J Pathol. 158 (2): 419-429; the Ambion RecoverAll™ Total Nucleic Acid Isolation Protocol (Ambion, Cat. No. AM1975, September 2008); the Maxwell® 16 FFPE Plus LEV DNA Purification Kit Technical Manual (Promega Literature #TM349, February 2011); the E.Z.N.A.® FFPE DNA Kit Handbook (OMEGA bio-tek, Norcross, GA, product numbers D3399-00, D3399-01, and D3399-02, June 2009); and the QIAamp® DNA FFPE Tissue Handbook (Qiagen, Cat. No. 37625, October 2007). For example, the RecoverAll™ Total Nucleic Acid Isolation Kit uses xylene at elevated temperatures to solubilize paraffin-embedded samples and a glass-fiber filter to capture nucleic acids. The Maxwell® 16 FFPE Plus LEV DNA Purification Kit is used with the Maxwell® 16 Instrument for purification of genomic DNA from 1 to 10 μm sections of FFPE tissue. DNA is purified using silica-clad paramagnetic particles (PMPs), and eluted in low elution volume. The E.Z.N.A.® FFPE DNA Kit uses a spin column and buffer system for isolation of genomic DNA. QIAamp® DNA FFPE Tissue Kit uses QIAamp® DNA Micro technology for purification of genomic and mitochondrial DNA.

In some instances, the disclosed methods may further comprise determining or acquiring a yield value for the nucleic acid extracted from the sample and comparing the determined value to a reference value. For example, if the determined or acquired value is less than the reference value, the nucleic acids may be amplified prior to proceeding with library construction. In some instances, the disclosed methods may further comprise determining or acquiring a value for the size (or average size) of nucleic acid fragments in the sample, and comparing the determined or acquired value to a reference value, e.g., a size (or average size) of at least 100, 200, 300, 400, 500, 600, 700, 800, 900, or 1000 base pairs (bps). In some instances, one or more parameters described herein may be adjusted or selected in response to this determination.

After isolation, the nucleic acids are typically dissolved in a slightly alkaline buffer, e.g., Tris-EDTA (TE) buffer, or in ultra-pure water. In some instances, the isolated nucleic acids (e.g., genomic DNA) may be fragmented or sheared by using any of a variety of techniques known to those of skill in the art. For example, genomic DNA can be fragmented by physical shearing methods, enzymatic cleavage methods, chemical cleavage methods, and other methods known to those of skill in the art. Methods for DNA shearing are described in Example 4 in International Patent Application Publication No. WO 2012/092426. In some instances, alternatives to DNA shearing methods can be used to avoid a ligation step during library preparation.

### F. Library Preparation

In some instances, the nucleic acids isolated from the sample may be used to construct a library (e.g., a nucleic acid library as described herein). In some instances, the nucleic acids are fragmented using any of the methods described above, optionally subjected to repair of chain end damage, and optionally ligated to synthetic adapters, primers, and/or barcodes (e.g., amplification primers, sequencing adapters, flow cell adapters, substrate adapters, sample barcodes or indexes, and/or unique molecular identifier sequences), size-selected (e.g., by preparative gel electrophoresis), and/or amplified (e.g., using PCR, a non-PCR amplification technique, or an isothermal amplification technique). In some instances, the fragmented and adapter-ligated group of nucleic acids is used without explicit size selection or amplification prior to hybridization-based selection of target sequences. In some instances, the nucleic acid is amplified by any of a variety of specific or non-specific nucleic acid amplification methods known to those of skill in the art. In some instances, the nucleic acids are amplified, e.g., by a whole-genome amplification method such as random-primed strand-displacement amplification. Examples of nucleic acid library preparation techniques for next-generation sequencing are described in, e.g., van Dijk, et al. (2014), Exp. Cell Research 322:12-20, and Illumina's genomic DNA sample preparation kit.

In some instances, the resulting nucleic acid library may contain all or substantially all of the complexity of the genome. The term “substantially all” in this context refers to the possibility that there can in practice be some unwanted loss of genome complexity during the initial steps of the procedure. The methods described herein also are useful in cases where the nucleic acid library comprises a portion of the genome, e.g., where the complexity of the genome is reduced by design. In some instances, any selected portion of the genome can be used with a method described herein. For example, in certain instances, the entire exome or a subset thereof is isolated. In some instances, the library may include at least 95%, 90%, 80%, 70%, 60%, 50%, 40%, 30%, 20%, 10%, or 5% of the genomic DNA. In some instances, the library may consist of cDNA copies of genomic DNA that includes copies of at least 95%, 90%, 80%, 70%, 60%, 50%, 40%, 30%, 20%, 10%, or 5% of the genomic DNA. In certain instances, the amount of nucleic acid used to generate the nucleic acid library may be less than 5 micrograms, less than 1 microgram, less than 500 ng, less than 200 ng, less than 100 ng, less than 50 ng, less than 10 ng, less than 5 ng, or less than 1 ng.

In some instances, a library (e.g., a nucleic acid library) includes a collection of nucleic acid molecules. As described herein, the nucleic acid molecules of the library can include a target nucleic acid molecule (e.g., a tumor nucleic acid molecule, a reference nucleic acid molecule and/or a control nucleic acid molecule; also referred to herein as a first, second and/or third nucleic acid molecule, respectively). The nucleic acid molecules of the library can be from a single subject or subjects. In some instances, a library can comprise nucleic acid molecules derived from more than one subject (e.g., 2, 3, 4, 5, 6, 7, 8, 9, 10, 20, 30 or more subjects). For example, two or more libraries from different subjects can be combined to form a library having nucleic acid molecules from more than one subject (where the nucleic acid molecules derived from each subject are optionally ligated to a unique sample barcode corresponding to a specific subject). In some instances, the subject is a human having, or at risk of having, a cancer or tumor.

In some instances, the library (or a portion thereof) may comprise one or more subgenomic intervals. In some instances, a subgenomic interval can be a single nucleotide position, e.g., a nucleotide position for which a variant at the position is associated (positively or negatively) with a tumor phenotype. In some instances, a subgenomic interval comprises more than one nucleotide position. Such instances include sequences of at least 2, 5, 10, 50, 100, 150, 250, or more than 250 nucleotide positions in length. Subgenomic intervals can comprise, e.g., one or more entire genes (or portions thereof), one or more exons or coding sequences (or portions thereof), one or more introns (or portion thereof), one or more microsatellite region (or portions thereof), or any combination thereof. A subgenomic interval can comprise all or a part of a fragment of a naturally occurring nucleic acid molecule, e.g., a genomic DNA molecule. For example, a subgenomic interval can correspond to a fragment of genomic DNA which is subjected to a sequencing reaction. In some instances, a subgenomic interval is a continuous sequence from a genomic source. In some instances, a subgenomic interval includes sequences that are not contiguous in the genome, e.g., subgenomic intervals in cDNA can include exon-exon junctions formed as a result of splicing. In some instances, the subgenomic interval comprises a tumor nucleic acid molecule. In some instances, the subgenomic interval comprises a non-tumor nucleic acid molecule.

### G. Targeting Gene Loci for Analysis

The methods described herein can be used in combination with, or as part of, a method for evaluating a plurality or set of subject intervals (e.g., target sequences), e.g., from a set of genomic loci (e.g., gene loci or fragments thereof), as described herein.

In some instances, the set of genomic loci evaluated by the disclosed methods comprises a plurality of, e.g., genes, which in mutant form, are associated with an effect on cell division, growth or survival, or are associated with a cancer, e.g., a cancer described herein.

In some instances, the set of gene loci evaluated by the disclosed methods comprises at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, or more than 100 gene loci.

In some instances, the selected gene loci (also referred to herein as target gene loci or target sequences), or fragments thereof, may include subject intervals comprising non-coding sequences, coding sequences, intragenic regions, or intergenic regions of the subject genome. For example, the subject intervals can include a non-coding sequence or fragment thereof (e.g., a promoter sequence, enhancer sequence, 5′ untranslated region (5′ UTR), 3′ untranslated region (3′ UTR), or a fragment thereof), a coding sequence of fragment thereof, an exon sequence or fragment thereof, an intron sequence or a fragment thereof.

### H. Target Capture Reagents

The methods described herein may comprise contacting a nucleic acid library with a plurality of target capture reagents in order to select and capture a plurality of specific target sequences (e.g., gene sequences or fragments thereof) for analysis. In some instances, a target capture reagent (i.e., a molecule which can bind to and thereby allow capture of a target molecule) is used to select the subject intervals to be analyzed. For example, a target capture reagent can be a bait molecule, e.g., a nucleic acid molecule (e.g., a DNA molecule or RNA molecule) which can hybridize to (i.e., is complementary to) a target molecule, and thereby allows capture of the target nucleic acid. In some instances, the target capture reagent, e.g., a bait molecule (or bait sequence), is a capture oligonucleotide (or capture probe). In some instances, the target nucleic acid is a genomic DNA molecule, an RNA molecule, a cDNA molecule derived from an RNA molecule, a microsatellite DNA sequence, and the like. In some instances, the target capture reagent is suitable for solution-phase hybridization to the target. In some instances, the target capture reagent is suitable for solid-phase hybridization to the target. In some instances, the target capture reagent is suitable for both solution-phase and solid-phase hybridization to the target. The design and construction of target capture reagents is described in more detail in, e.g., International Patent Application Publication No. WO 2020/236941, the entire content of which is incorporated herein by reference.

The methods described herein provide for optimized sequencing of a large number of genomic loci (e.g., genes or gene products (e.g., mRNA), microsatellite loci, etc.) from samples (e.g., cancerous tissue specimens, liquid biopsy samples, and the like) from one or more subjects by the appropriate selection of target capture reagents to select the target nucleic acid molecules to be sequenced. In some instances, a target capture reagent may hybridize to a specific target locus, e.g., a specific target gene locus or fragment thereof. In some instances, a target capture reagent may hybridize to a specific group of target loci, e.g., a specific group of gene loci or fragments thereof. In some instances, a plurality of target capture reagents comprising a mix of target-specific and/or group-specific target capture reagents may be used.

In some instances, the number of target capture reagents (e.g., bait molecules) in the plurality of target capture reagents (e.g., a bait set) contacted with a nucleic acid library to capture a plurality of target sequences for nucleic acid sequencing is greater than 10, greater than 50, greater than 100, greater than 200, greater than 300, greater than 400, greater than 500, greater than 600, greater than 700, greater than 800, greater than 900, greater than 1,000, greater than 1,250, greater than 1,500, greater than 1,750, greater than 2,000, greater than 3,000, greater than 4,000, greater than 5,000, greater than 10,000, greater than 25,000, or greater than 50,000.

In some instances, the overall length of the target capture reagent sequence can be between about 70 nucleotides and 1000 nucleotides. In one instance, the target capture reagent length is between about 100 and 300 nucleotides, 110 and 200 nucleotides, or 120 and 170 nucleotides, in length. In addition to those mentioned above, intermediate oligonucleotide lengths of about 70, 80, 90, 100, 110, 120, 130, 140, 150, 160, 170, 180, 190, 200, 210, 220, 230, 240, 250, 300, 400, 500, 600, 700, 800, and 900 nucleotides in length can be used in the methods described herein. In some instances, oligonucleotides of about 70, 80, 90, 100, 110, 120, 130, 140, 150, 160, 170, 180, 190, 200, 210, 220, or 230 bases can be used.

In some instances, each target capture reagent sequence can include: (i) a target-specific capture sequence (e.g., a gene locus or microsatellite locus-specific complementary sequence), (ii) an adapter, primer, barcode, and/or unique molecular identifier sequence, and (iii) universal tails on one or both ends. As used herein, the term “target capture reagent” can refer to the target-specific target capture sequence or to the entire target capture reagent oligonucleotide including the target-specific target capture sequence.

In some instances, the target-specific capture sequences in the target capture reagents are between about 40 nucleotides and 1000 nucleotides in length. In some instances, the target-specific capture sequence is between about 70 nucleotides and 300 nucleotides in length. In some instances, the target-specific sequence is between about 100 nucleotides and 200 nucleotides in length. In yet other instances, the target-specific sequence is between about 120 nucleotides and 170 nucleotides in length, typically 120 nucleotides in length. Intermediate lengths in addition to those mentioned above also can be used in the methods described herein, such as target-specific sequences of about 40, 50, 60, 70, 80, 90, 100, 110, 120, 130, 140, 150, 160, 170, 180, 190, 200, 210, 220, 230, 240, 250, 300, 400, 500, 600, 700, 800, and 900 nucleotides in length, as well as target-specific sequences of lengths between the above-mentioned lengths.

In some instances, the target capture reagent may be designed to select a subject interval containing one or more rearrangements, e.g., an intron containing a genomic rearrangement. In such instances, the target capture reagent is designed such that repetitive sequences are masked to increase the selection efficiency. In those instances where the rearrangement has a known juncture sequence, complementary target capture reagents can be designed to recognize the juncture sequence to increase the selection efficiency.

In some instances, the disclosed methods may comprise the use of target capture reagents designed to capture two or more different target categories, each category having a different target capture reagent design strategy. In some instances, the hybridization-based capture methods and target capture reagent compositions disclosed herein may provide for the capture and homogeneous coverage of a set of target sequences, while minimizing coverage of genomic sequences outside of the targeted set of sequences. In some instances, the target sequences may include the entire exome of genomic DNA or a selected subset thereof. In some instances, the target sequences may include, e.g., a large chromosomal region (e.g., a whole chromosome arm). The methods and compositions disclosed herein provide different target capture reagents for achieving different sequencing depths and patterns of coverage for complex sets of target nucleic acid sequences.

Typically, DNA molecules are used as target capture reagent sequences, although RNA molecules can also be used. In some instances, a DNA molecule target capture reagent can be single stranded DNA (ssDNA) or double-stranded DNA (dsDNA). In some instances, an RNA-DNA duplex is more stable than a DNA-DNA duplex and therefore provides for potentially better capture of nucleic acids.

In some instances, the disclosed methods comprise providing a selected set of nucleic acid molecules (e.g., a library catch) captured from one or more nucleic acid libraries. For example, the method may comprise: providing one or a plurality of nucleic acid libraries, each comprising a plurality of nucleic acid molecules (e.g., a plurality of target nucleic acid molecules and/or reference nucleic acid molecules) extracted from one or more samples from one or more subjects; contacting the one or a plurality of libraries (e.g., in a solution-based hybridization reaction) with one, two, three, four, five, or more than five pluralities of target capture reagents (e.g., oligonucleotide target capture reagents) to form a hybridization mixture comprising a plurality of target capture reagent/nucleic acid molecule hybrids; separating the plurality of target capture reagent/nucleic acid molecule hybrids from said hybridization mixture, e.g., by contacting said hybridization mixture with a binding entity that allows for separation of said plurality of target capture reagent/nucleic acid molecule hybrids from the hybridization mixture, thereby providing a library catch (e.g., a selected or enriched subgroup of nucleic acid molecules from the one or a plurality of libraries).

In some instances, the disclosed methods may further comprise amplifying the library catch (e.g., by performing PCR). In other instances, the library catch is not amplified.

In some instances, the target capture reagents can be part of a kit which can optionally comprise instructions, standards, buffers or enzymes or other reagents.

### 1. Hybridization Conditions

As noted above, the methods disclosed herein may include the step of contacting the library (e.g., the nucleic acid library) with a plurality of target capture reagents to provide a selected library target nucleic acid sequences (i.e., the library catch). The contacting step can be effected in, e.g., solution-based hybridization. In some instances, the method includes repeating the hybridization step for one or more additional rounds of solution-based hybridization. In some instances, the method further includes subjecting the library catch to one or more additional rounds of solution-based hybridization with the same or a different collection of target capture reagents.

In some instances, the contacting step is effected using a solid support, e.g., an array. Suitable solid supports for hybridization are described in, e.g., Albert, T. J. et al. (2007) Nat. Methods 4 (11): 903-5; Hodges, E. et al. (2007) Nat. Genet. 39 (12): 1522-7; and Okou, D. T. et al. (2007) Nat. Methods 4 (11): 907-9, the contents of which are incorporated herein by reference in their entireties.

Hybridization methods that can be adapted for use in the methods herein are described in the art, e.g., as described in International Patent Application Publication No. WO 2012/092426. Methods for hybridizing target capture reagents to a plurality of target nucleic acids are described in more detail in, e.g., International Patent Application Publication No. WO 2020/236941, the entire content of which is incorporated herein by reference.

### J. Sequencing Methods

The methods and systems disclosed herein can be used in combination with, or as part of, a method or system for sequencing nucleic acids (e.g., a next-generation sequencing system) to generate a plurality of sequence reads that overlap one or more gene loci within a subgenomic interval in the sample and thereby determine, e.g., gene allele sequences at a plurality of gene loci. “Next-generation sequencing” (or “NGS”) as used herein may also be referred to as “massively parallel sequencing”, and refers to any sequencing method that determines the nucleotide sequence of either individual nucleic acid molecules (e.g., as in single molecule sequencing) or clonally expanded proxies for individual nucleic acid molecules in a high throughput fashion (e.g., wherein greater than 103, 104, 105 or more than 105 molecules are sequenced simultaneously).

Next-generation sequencing methods are known in the art, and are described in, e.g., Metzker, M. (2010) Nature Biotechnology Reviews 11:31-46, which is incorporated herein by reference. Other examples of sequencing methods suitable for use when implementing the methods and systems disclosed herein are described in, e.g., International Patent Application Publication No. WO 2012/092426. In some instances, the sequencing may comprise, for example, whole genome sequencing (WGS), whole exome sequencing, targeted sequencing, or direct sequencing. In some instances, sequencing may be performed using, e.g., Sanger sequencing. In some instances, the sequencing may comprise a paired-end sequencing technique that allows both ends of a fragment to be sequenced and generates high-quality, alignable sequence data for detection of, e.g., genomic rearrangements, repetitive sequence elements, gene fusions, and novel transcripts.

The disclosed methods and systems may be implemented using sequencing platforms such as the Roche 454, Illumina Solexa, ABI-SOLID, ION Torrent, Complete Genomics, Pacific Bioscience, Helicos, and/or the Polonator platform. In some instances, sequencing may comprise Illumina MiSeq sequencing. In some instances, sequencing may comprise Illumina HiSeq sequencing. In some instances, sequencing may comprise Illumina NovaSeq sequencing. Optimized methods for sequencing a large number of target genomic loci in nucleic acids extracted from a sample are described in more detail in, e.g., International Patent Application Publication No. WO 2020/236941, the entire content of which is incorporated herein by reference.

In certain instances, the disclosed methods comprise one or more of the steps of: (a) acquiring a library comprising a plurality of normal and/or tumor nucleic acid molecules from a sample; (b) simultaneously or sequentially contacting the library with one, two, three, four, five, or more than five pluralities of target capture reagents under conditions that allow hybridization of the target capture reagents to the target nucleic acid molecules, thereby providing a selected set of captured normal and/or tumor nucleic acid molecules (i.e., a library catch); (c) separating the selected subset of the nucleic acid molecules (e.g., the library catch) from the hybridization mixture, e.g., by contacting the hybridization mixture with a binding entity that allows for separation of the target capture reagent/nucleic acid molecule hybrids from the hybridization mixture, (d) sequencing the library catch to acquiring a plurality of reads (e.g., sequence reads) that overlap one or more subject intervals (e.g., one or more target sequences) from said library catch that may comprise a mutation (or alteration), e.g., a variant sequence comprising a somatic mutation or germline mutation; (e) aligning said sequence reads using an alignment method as described elsewhere herein; and/or (f) assigning a nucleotide value for a nucleotide position in the subject interval (e.g., calling a mutation using, e.g., a Bayesian method or other method described herein) from one or more sequence reads of the plurality.

In some instances, acquiring sequence reads for one or more subject intervals may comprise sequencing at least 1, at least 5, at least 10, at least 20, at least 30, at least 40, at least 50, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1,000, at least 1,250, at least 1,500, at least 1,750, at least 2,000, at least 2,250, at least 2,500, at least 2,750, at least 3,000, at least 3,500, at least 4,000, at least 4,500, or at least 5,000 loci, e.g., genomic loci, gene loci, microsatellite loci, etc. In some instances, acquiring a sequence read for one or more subject intervals may comprise sequencing a subject interval for any number of loci within the range described in this paragraph, e.g., for at least 2,850 gene loci.

In some instances, acquiring a sequence read for one or more subject intervals comprises sequencing a subject interval with a sequencing method that provides a sequence read length (or average sequence read length) of at least 20 bases, at least 30 bases, at least 40 bases, at least 50 bases, at least 60 bases, at least 70 bases, at least 80 bases, at least 90 bases, at least 100 bases, at least 120 bases, at least 140 bases, at least 160 bases, at least 180 bases, at least 200 bases, at least 220 bases, at least 240 bases, at least 260 bases, at least 280 bases, at least 300 bases, at least 320 bases, at least 340 bases, at least 360 bases, at least 380 bases, or at least 400 bases. In some instances, acquiring a sequence read for the one or more subject intervals may comprise sequencing a subject interval with a sequencing method that provides a sequence read length (or average sequence read length) of any number of bases within the range described in this paragraph, e.g., a sequence read length (or average sequence read length) of 56 bases.

In some instances, acquiring a sequence read for one or more subject intervals may comprise sequencing with at least 100× or more coverage (or depth) on average. In some instances, acquiring a sequence read for one or more subject intervals may comprise sequencing with at least 100×, at least 150×, at least 200×, at least 250×, at least 500×, at least 750×, at least 1,000×, at least 1,500×, at least 2,000×, at least 2,500×, at least 3,000×, at least 3,500×, at least 4,000×, at least 4,500×, at least 5,000×, at least 5,500×, or at least 6,000× or more coverage (or depth) on average. In some instances, acquiring a sequence read for one or more subject intervals may comprise sequencing with an average coverage (or depth) having any value within the range of values described in this paragraph, e.g., at least 160×.

In some instances, acquiring a read for the one or more subject intervals comprises sequencing with an average sequencing depth having any value ranging from at least 100× to at least 6,000× for greater than about 90%, 92%, 94%, 95%, 96%, 97%, 98%, or 99% of the gene loci sequenced. For example, in some instances acquiring a read for the subject interval comprises sequencing with an average sequencing depth of at least 125× for at least 99% of the gene loci sequenced. As another example, in some instances acquiring a read for the subject interval comprises sequencing with an average sequencing depth of at least 4,100× for at least 95% of the gene loci sequenced.

In some instances, the relative abundance of a nucleic acid species in the library can be estimated by counting the relative number of occurrences of their cognate sequences (e.g., the number of sequence reads for a given cognate sequence) in the data generated by the sequencing experiment.

In some instances, the disclosed methods and systems provide nucleotide sequences for a set of subject intervals (e.g., gene loci), as described herein. In certain instances, the sequences are provided without using a method that includes a matched normal control (e.g., a wild-type control) and/or a matched tumor control (e.g., primary versus metastatic).

In some instances, the level of sequencing depth as used herein (e.g., an X-fold level of sequencing depth) refers to the number of reads (e.g., unique reads) obtained after detection and removal of duplicate reads (e.g., PCR duplicate reads). In other instances, duplicate reads are evaluated, e.g., to support detection of copy number alteration (CNAs).

### K. Alignment

Alignment is the process of matching a read with a location, e.g., a genomic location or locus. In some instances, NGS reads may be aligned to a known reference sequence (e.g., a wild-type sequence). In some instances, NGS reads may be assembled de novo. Methods of sequence alignment for NGS reads are described in, e.g., Trapnell, C. and Salzberg, S. L. Nature Biotech., 2009, 27:455-457. Examples of de novo sequence assemblies are described in, e.g., Warren R., et al., Bioinformatics, 2007, 23:500-501; Butler, J. et al., Genome Res., 2008, 18:810-820; and Zerbino, D. R. and Birncy, E., Genome Res., 2008, 18:821-829. Optimization of sequence alignment is described in the art, e.g., as set out in International Patent Application Publication No. WO 2012/092426. Additional description of sequence alignment methods is provided in, e.g., International Patent Application Publication No. WO 2020/236941, the entire content of which is incorporated herein by reference.

Misalignment (e.g., the placement of base-pairs from a short read at incorrect locations in the genome), e.g., misalignment of reads due to sequence context (e.g., the presence of repetitive sequence) around an actual cancer mutation can lead to reduction in sensitivity of mutation detection, can lead to a reduction in sensitivity of mutation detection, as reads for the alternate allele may be shifted off the histogram peak of alternate allele reads. Other examples of sequence context that may cause misalignment include short-tandem repeats, interspersed repeats, low complexity regions, insertions-deletions (indels), and paralogs. If the problematic sequence context occurs where no actual mutation is present, misalignment may introduce artifactual reads of “mutated” alleles by placing reads of actual reference genome base sequences at the wrong location. Because mutation-calling algorithms for multigene analysis should be sensitive to even low-abundance mutations, sequence misalignments may increase false positive discovery rates and/or reduce specificity.

In some instances, the methods and systems disclosed herein may integrate the use of multiple, individually-tuned, alignment methods or algorithms to optimize base-calling performance in sequencing methods, particularly in methods that rely on massively parallel sequencing of a large number of diverse genetic events at a large number of diverse genomic loci. In some instances, the disclosed methods and systems may comprise the use of one or more global alignment algorithms. In some instances, the disclosed methods and systems may comprise the use of one or more local alignment algorithms. Examples of alignment algorithms that may be used include, but are not limited to, the Burrows-Wheeler Alignment (BWA) software bundle (see, e.g., Li, et al. (2009), “Fast and Accurate Short Read Alignment with Burrows-Wheeler Transform”, Bioinformatics 25:1754-60; Li, et al. (2010), Fast and Accurate Long-Read Alignment with Burrows-Wheeler Transform “, Bioinformatics epub. PMID:

20080505), the Smith-Waterman algorithm (see, e.g., Smith, et al. (1981), “Identification of Common Molecular Subsequences”, J. Molecular Biology 147 (1): 195-197), the Striped Smith-Waterman algorithm (see, e.g., Farrar (2007), “Striped Smith-Waterman Speeds Database Searches Six Times Over Other SIMD Implementations”, Bioinformatics 23 (2): 156-161), the Needleman-Wunsch algorithm (Needleman, et al. (1970) “A General Method Applicable to the Search for Similarities in the Amino Acid Sequence of Two Proteins”, J. Molecular Biology 48 (3): 443-53), or any combination thereof.

In some instances, the methods and systems disclosed herein may also comprise the use of a sequence assembly algorithm, e.g., the Arachne sequence assembly algorithm (see, e.g., Batzoglou, et al. (2002), “ARACHNE: A Whole-Genome Shotgun Assembler”, Genome Res. 12:177-189).

In some instances, the alignment method used to analyze sequence reads is not individually customized or tuned for detection of different variants (e.g., point mutations, insertions, deletions, and the like) at different genomic loci. In some instances, different alignment methods are used to analyze reads that are individually customized or tuned for detection of at least a subset of the different variants detected at different genomic loci. In some instances, different alignment methods are used to analyze reads that are individually customized or tuned to detect each different variant at different genomic loci. In some instances, tuning can be a function of one or more of: (i) the genetic locus (e.g., gene loci, microsatellite locus, or other subject interval) being sequenced, (ii) the tumor type associated with the sample, (iii) the variant being sequenced, or (iv) a characteristic of the sample or the subject. The selection or use of alignment conditions that are individually tuned to a number of specific subject intervals to be sequenced allows optimization of speed, sensitivity, and specificity. The method is particularly effective when the alignment of reads for a relatively large number of diverse subject intervals are optimized. In some instances, the method includes the use of an alignment method optimized for rearrangements in combination with other alignment methods optimized for subject intervals not associated with rearrangements.

In some instances, the methods disclosed herein further comprise selecting or using an alignment method for analyzing, e.g., aligning, a sequence read, wherein said alignment method is a function of, is selected responsive to, or is optimized for, one or more of: (i) tumor type, e.g., the tumor type in the sample; (ii) the location (e.g., a gene locus) of the subject interval being sequenced; (iii) the type of variant (e.g., a point mutation, insertion, deletion, substitution, copy number variation (CNV), rearrangement, or fusion) in the subject interval being sequenced; (iv) the site (e.g., nucleotide position) being analyzed; (v) the type of sample (e.g., a sample described herein); and/or (vi) adjacent sequence(s) in or near the subject interval being evaluated (e.g., according to the expected propensity thereof for misalignment of the subject interval due to, e.g., the presence of repeated sequences in or near the subject interval).

In some instances, the methods disclosed herein allow for the rapid and efficient alignment of troublesome reads, e.g., a read having a rearrangement. Thus, in some instances where a read for a subject interval comprises a nucleotide position with a rearrangement, e.g., a translocation, the method can comprise using an alignment method that is appropriately tuned and that includes: (i) selecting a rearrangement reference sequence for alignment with a read, wherein said rearrangement reference sequence aligns with a rearrangement (in some instances, the reference sequence is not identical to the genomic rearrangement); and (ii) comparing, e.g., aligning, a read with said rearrangement reference sequence.

In some instances, alternative methods may be used to align troublesome reads. These methods are particularly effective when the alignment of reads for a relatively large number of diverse subject intervals is optimized. By way of example, a method of analyzing a sample can comprise: (i) performing a comparison (e.g., an alignment comparison) of a read using a first set of parameters (e.g., using a first mapping algorithm, or by comparison with a first reference sequence), and determining if said read meets a first alignment criterion (e.g., the read can be aligned with said first reference sequence, e.g., with less than a specific number of mismatches); (ii) if said read fails to meet the first alignment criterion, performing a second alignment comparison using a second set of parameters, (e.g., using a second mapping algorithm, or by comparison with a second reference sequence); and (iii) optionally, determining if said read meets said second criterion (e.g., the read can be aligned with said second reference sequence, e.g., with less than a specific number of mismatches), wherein said second set of parameters comprises use of, e.g., said second reference sequence, which, compared with said first set of parameters, is more likely to result in an alignment with a read for a variant (e.g., a rearrangement, insertion, deletion, or translocation).

In some instances, the alignment of sequence reads in the disclosed methods may be combined with a mutation calling method as described elsewhere herein. As discussed herein, reduced sensitivity for detecting actual mutations may be addressed by evaluating the quality of alignments (manually or in an automated fashion) around expected mutation sites in the genes or genomic loci (e.g., gene loci) being analyzed. In some instances, the sites to be evaluated can be obtained from databases of the human genome (e.g., the HG19 human reference genome) or cancer mutations (e.g., COSMIC). Regions that are identified as problematic can be remedied with the use of an algorithm selected to give better performance in the relevant sequence context, e.g., by alignment optimization (or re-alignment) using slower, but more accurate alignment algorithms such as Smith-Waterman alignment. In cases where general alignment algorithms cannot remedy the problem, customized alignment approaches may be created by, e.g., adjustment of maximum difference mismatch penalty parameters for genes with a high likelihood of containing substitutions; adjusting specific mismatch penalty parameters based on specific mutation types that are common in certain tumor types (e.g. CàT in melanoma); or adjusting specific mismatch penalty parameters based on specific mutation types that are common in certain sample types (e.g. substitutions that are common in FFPE).

Reduced specificity (increased false positive rate) in the evaluated subject intervals due to misalignment can be assessed by manual or automated examination of all mutation calls in the sequencing data. Those regions found to be prone to spurious mutation calls due to misalignment can be subjected to alignment remedies as discussed above. In cases where no algorithmic remedy is found possible, “mutations” from the problem regions can be classified or screened out from the panel of targeted loci.

### L. Mutation Calling

Base calling refers to the raw output of a sequencing device, e.g., the determined sequence of nucleotides in an oligonucleotide molecule. Mutation calling refers to the process of selecting a nucleotide value, e.g., A, G, T, or C, for a given nucleotide position being sequenced. Typically, the sequence reads (or base calling) for a position will provide more than one value, e.g., some reads will indicate a T and some will indicate a G. Mutation calling is the process of assigning a correct nucleotide value, e.g., one of those values, to the sequence. Although it is referred to as “mutation” calling, it can be applied to assign a nucleotide value to any nucleotide position, e.g., positions corresponding to mutant alleles, wild-type alleles, alleles that have not been characterized as either mutant or wild-type, or to positions not characterized by variability.

In some instances, the disclosed methods may comprise the use of customized or tuned mutation calling algorithms or parameters thereof to optimize performance when applied to sequencing data, particularly in methods that rely on massively parallel sequencing of a large number of diverse genetic events at a large number of diverse genomic loci (e.g., gene loci, microsatellite regions, etc.) in samples, e.g., samples from a subject having cancer. Optimization of mutation calling is described in the art, e.g., as set out in International Patent Application Publication No. WO 2012/092426.

Methods for mutation calling can include one or more of the following: making independent calls based on the information at each position in the reference sequence (e.g., examining the sequence reads; examining the base calls and quality scores; calculating the probability of observed bases and quality scores given a potential genotype; and assigning genotypes (e.g., using Bayes' rule)); removing false positives (e.g., using depth thresholds to reject SNPs with read depth much lower or higher than expected; local realignment to remove false positives due to small indels); and performing linkage disequilibrium (LD)/imputation-based analysis to refine the calls.

Equations used to calculate the genotype likelihood associated with a specific genotype and position are described in, e.g., Li, H. and Durbin, R. Bioinformatics, 2010; 26 (5): 589-95. The prior expectation for a particular mutation in a certain cancer type can be used when evaluating samples from that cancer type. Such likelihood can be derived from public databases of cancer mutations, e.g., Catalogue of Somatic Mutation in Cancer (COSMIC), HGMD (Human Gene Mutation Database), The SNP Consortium, Breast Cancer Mutation Data Base (BIC), and Breast Cancer Gene Database (BCGD).

Examples of LD/imputation based analysis are described in, e.g., Browning, B. L. and Yu, Z. Am. J. Hum. Genet. 2009, 85 (6): 847-61. Examples of low-coverage SNP calling methods are described in, e.g., Li, Y., et al., Annu. Rev. Genomics Hum. Genet. 2009, 10:387-406.

After alignment, detection of substitutions can be performed using a mutation calling method (e.g., a Bayesian mutation calling method) which is applied to each base in each of the subject intervals, e.g., exons of a gene or other locus to be evaluated, where presence of alternate alleles is observed. This method will compare the probability of observing the read data in the presence of a mutation with the probability of observing the read data in the presence of base-calling error alone. Mutations can be called if this comparison is sufficiently strongly supportive of the presence of a mutation.

An advantage of a Bayesian mutation-detection approach is that the comparison of the probability of the presence of a mutation with the probability of base-calling error alone can be weighted by a prior expectation of the presence of a mutation at the site. If some reads of an alternate allele are observed at a frequently mutated site for the given cancer type, then presence of a mutation may be confidently called even if the amount of evidence of mutation does not meet the usual thresholds. This flexibility can then be used to increase detection sensitivity for even rarer mutations/lower purity samples, or to make the test more robust to decreases in read coverage. The likelihood of a random base-pair in the genome being mutated in cancer is ˜ le-6. The likelihood of specific mutations occurring at many sites in, for example, a typical multigenic cancer genome panel can be orders of magnitude higher. These likelihoods can be derived from public databases of cancer mutations (e.g., COSMIC).

Indel calling is a process of finding bases in the sequencing data that differ from the reference sequence by insertion or deletion, typically including an associated confidence score or statistical evidence metric. Methods of indel calling can include the steps of identifying candidate indels, calculating genotype likelihood through local re-alignment, and performing LD-based genotype inference and calling. Typically, a Bayesian approach is used to obtain potential indel candidates, and then these candidates are tested together with the reference sequence in a Bayesian framework.

Algorithms to generate candidate indels are described in, e.g., McKenna, A., et al., Genome Res. 2010; 20 (9): 1297-303; Ye, K., et al., Bioinformatics, 2009; 25 (21): 2865-71; Lunter, G., and Goodson, M., Genome Res. 2011; 21 (6): 936-9; and Li, H., et al. (2009), Bioinformatics 25 (16): 2078-9.

Methods for generating indel calls and individual-level genotype likelihoods include, e.g., the Dindel algorithm (Albers, C.A., et al., Genome Res. 2011; 21 (6): 961-73). For example, the Bayesian EM algorithm can be used to analyze the reads, make initial indel calls, and generate genotype likelihoods for each candidate indel, followed by imputation of genotypes using, e.g., QCALL (Le S. Q. and Durbin R. Genome Res. 2011; 21 (6): 952-60). Parameters, such as prior expectations of observing the indel can be adjusted (e.g., increased or decreased), based on the size or location of the indels.

Methods have been developed that address limited deviations from allele frequencies of 50% or 100% for the analysis of cancer DNA. (see, e.g., SNVMix-Bioinformatics. 2010 Mar. 15; 26 (6): 730-736.) Methods disclosed herein, however, allow consideration of the possibility of the presence of a mutant allele at frequencies (or allele fractions) ranging from 1% to 100% (i.e., allele fractions ranging from 0.01 to 1.0), and especially at levels lower than 50%. This approach is particularly important for the detection of mutations in, for example, low-purity FFPE samples of natural (multi-clonal) tumor DNA.

In some instances, the mutation calling method used to analyze sequence reads is not individually customized or fine-tuned for detection of different mutations at different genomic loci. In some instances, different mutation calling methods are used that are individually customized or fine-tuned for at least a subset of the different mutations detected at different genomic loci. In some instances, different mutation calling methods are used that are individually customized or fine-tuned for each different mutant detected at each different genomic loci. The customization or tuning can be based on one or more of the factors described herein, e.g., the type of cancer in a sample, the gene or locus in which the subject interval to be sequenced is located, or the variant to be sequenced. This selection or use of mutation calling methods individually customized or fine-tuned for a number of subject intervals to be sequenced allows for optimization of speed, sensitivity and specificity of mutation calling.

In some instances, a nucleotide value is assigned for a nucleotide position in each of X unique subject intervals using a unique mutation calling method, and X is at least 2, at least 3, at least 4, at least 5, at least 10, at least 15, at least 20, at least 30, at least 40, at least 50, at least 60, at least 70, at least 80, at least 90, at least 100, at least 200, at least 300, at least 400, at least 500, at least 1000, at least 1500, at least 2000, at least 2500, at least 3000, at least 3500, at least 4000, at least 4500, at least 5000, or greater. The calling methods can differ, and thereby be unique, e.g., by relying on different Bayesian prior values.

In some instances, assigning said nucleotide value is a function of a value which is or represents the prior (e.g., literature) expectation of observing a read showing a variant, e.g., a mutation, at said nucleotide position in a tumor of type.

In some instances, the method comprises assigning a nucleotide value (e.g., calling a mutation) for at least 10, 20, 40, 50, 60, 70, 80, 90, 100, 200, 300, 400, 500, 600, 700, 800, 900, or 1,000 nucleotide positions, wherein each assignment is a function of a unique value (as opposed to the value for the other assignments) which is or represents the prior (e.g., literature) expectation of observing a read showing a variant, e.g., a mutation, at said nucleotide position in a tumor of type.

In some instances, assigning said nucleotide value is a function of a set of values which represent the probabilities of observing a read showing said variant at said nucleotide position if the variant is present in the sample at a specified frequency (e.g., 1%, 5%, 10%, etc.) and/or if the variant is absent (e.g., observed in the reads due to base-calling error alone).

In some instances, the mutation calling methods described herein can include the following: (a) acquiring, for a nucleotide position in each of said X subject intervals: (i) a first value which is or represents the prior (e.g., literature) expectation of observing a read showing a variant, e.g., a mutation, at said nucleotide position in a tumor of type X; and (ii) a second set of values which represent the probabilities of observing a read showing said variant at said nucleotide position if the variant is present in the sample at a frequency (e.g., 1%, 5%, 10%, etc.) and/or if the variant is absent (e.g., observed in the reads due to base-calling error alone); and (b) responsive to said values, assigning a nucleotide value (e.g., calling a mutation) from said reads for each of said nucleotide positions by weighing, e.g., by a Bayesian method described herein, the comparison among the values in the second set using the first value (e.g., computing the posterior probability of the presence of a mutation), thereby analyzing said sample.

Additional description of mutation calling methods is provided in, e.g., International Patent Application Publication No. WO 2020/236941, the entire content of which is incorporated herein by reference.

### III. Methods of Use of Risk Scores

Once a risk score has been determined for a subject having cancer, and compared to a pre-determined threshold, the disclosure provides for therapies responsive to said comparison. The subject may be any of the subjects described in Section II. C. of the disclosure. Additionally, the cancer may be any of the cancers described in Section II. D. of the disclosure.

### A. Chemotherapies

Certain aspects of the present disclosure relate to chemotherapies.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammall and calicheamicin omegall); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenishers such as folinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; ctoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylomithine (DMFO); retinoids, such as retinoic acid; capecitabine; carboplatin, procarbazine, plicomycin, gemcitabine, navelbine, farnesyl-protein tansferase inhibitors, transplatinum, and pharmaceutically acceptable salts, acids, or derivatives of any of the above.

In some instances, the chemotherapy comprises FOLFIRINOX, gemcitabine plus albumin-bound paclitaxel, gemcitabine, capecitabine, fluorouacil plus irinotecan liposomal and leucovorin, FOLFIRI, or capecitabine plus gemcitabine. In some instances, the chemotherapy is FOLFIRINOX. In some instances, the chemotherapy is gemcitabine plus albumin-bound paclitaxel (G+P).

In some instances, the methods of the disclosure further comprise treating a subject with the chemotherapy. In some instances, the chemotherapy comprises administered as a monotherapy. In some instances, the chemotherapy is administered in combination with a second anti-cancer therapy. In some instances, the methods further comprise treating the subject with an additional anticancer therapy.

### B. Immuno-Oncology Therapy

Certain aspects of the present disclosure relate to immuno-oncology (IO) therapies.

In some instances the IO therapy comprises an immune checkpoint inhibitor. As is known in the art, a checkpoint inhibitor targets at least one immune checkpoint protein to alter the regulation of an immune response. Immune checkpoint proteins include, e.g., CTLA4, PD-L1, PD-1, PD-L2, VISTA, B7-H2, B7-H3, B7-H4, B7-H6, 2B4, ICOS, HVEM, CEACAM, LAIR1, CD80, CD86, CD276, VTCNI, MHC class I, MHC class II, GALS, adenosine, TGFR, CSFIR, MICA/B, arginase, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, LAG-3, BTLA, IDO, OX40, and A2aR. In some instances, molecules involved in regulating immune checkpoints include, but are not limited to: PD-1 (CD279), PD-L1 (B7-H1, CD274), PD-L2 (B7-CD, CD273), CTLA-4 (CD152), HVEM, BTLA (CD272), a killer-cell immunoglobulin-like receptor (KIR), LAG-3 (CD223), TIM-3 (HAVCR2), CEACAM, CEACAM-1, CEACAM-3, CEACAM-5, GAL9, VISTA (PD-1H), TIGIT, LAIR1, CD160, 2B4, TGFRbeta, A2AR, GITR (CD357), CD80 (B7-1), CD86 (B7-2), CD276 (B7-H3), VTCNI (B7-H4), MHC class I, MHC class II, GALS, adenosine, TGFR, B7-H1, OX40 (CD134), CD94 (KLRD1), CD137 (4-1BB), CD137L (4-1BBL), CD40, IDO, CSFIR, CD40L, CD47, CD70 (CD27L), CD226, HHLA2, ICOS (CD278), ICOSL (CD275), LIGHT (TNFSF14, CD258), NKG2a, NKG2d, OX40L (CD134L), PVR (NECL5, CD155), SIRPa, MICA/B, and/or arginase. In some instances, an immune checkpoint inhibitor (i.e., a checkpoint inhibitor) decreases the activity of a checkpoint protein that negatively regulates immune cell function, e.g., in order to enhance T cell activation and/or an anti-cancer immune response. In other instances, a checkpoint inhibitor increases the activity of a checkpoint protein that positively regulates immune cell function, e.g., in order to enhance T cell activation and/or an anti-cancer immune response. In some instances, the checkpoint inhibitor is an antibody. Examples of checkpoint inhibitors include, without limitation, a PD-1 axis binding antagonist, a PD-L1 axis binding antagonist (e.g., an anti-PD-L1 antibody, e.g., atezolizumab (MPDL3280A)), an antagonist directed against a co-inhibitory molecule (e.g., a CTLA4 antagonist (e.g., an anti-CTLA4 antibody), a TIM-3 antagonist (e.g., an anti-TIM-3 antibody), or a LAG-3 antagonist (e.g., an anti-LAG-3 antibody)), or any combination thereof. In some instances, the immune checkpoint inhibitors comprise drugs such as small molecules, recombinant forms of ligand or receptors, or antibodies, such as human antibodies (see, e.g., International Patent Publication WO2015016718; Pardoll, Nat Rev Cancer, 12 (4): 252-64, 2012; both incorporated herein by reference). In some instances, known inhibitors of immune checkpoint proteins or analogs thereof may be used, in particular chimerized, humanized or human forms of antibodies may be used.

In some instances according to any of the instances described herein, the immune checkpoint inhibitor comprises a PD-1 antagonist/inhibitor or a PD-L1 antagonist/inhibitor.

In some instances, the checkpoint inhibitor is a PD-L1 axis binding antagonist, e.g., a PD-1 binding antagonist, a PD-L1 binding antagonist, or a PD-L2 binding antagonist. PD-1 (programmed death 1) is also referred to in the art as “programmed cell death 1,” “PDCD1,” “CD279,” and “SLEB2.” An exemplary human PD-1 is shown in UniProtKB/Swiss-Prot Accession No. Q15116. PD-L1 (programmed death ligand 1) is also referred to in the art as “programmed cell death 1 ligand 1,” “PDCD1 LG1,” “CD274,” “B7-H,” and “PDL1.” An exemplary human PD-L1 is shown in UniProtKB/Swiss-Prot Accession No.Q9NZQ7.1. PD-L2 (programmed death ligand 2) is also referred to in the art as “programmed cell death 1 ligand 2,” “PDCD1 LG2,” “CD273,” “B7-DC,” “Btdc,” and “PDL2.” An exemplary human PD-L2 is shown in UniProtKB/Swiss-Prot Accession No. Q9BQ51. In some instances, PD-1, PD-L1, and PD-L2 are human PD-1, PD-L1 and PD-L2.

In some instances, the PD-1 binding antagonist/inhibitor is a molecule that inhibits the binding of PD-1 to its ligand binding partners. In a specific instance, the PD-1 ligand binding partners are PD-L1 and/or PD-L2. In another instance, a PD-L1 binding antagonist/inhibitor is a molecule that inhibits the binding of PD-L1 to its binding ligands. In a specific instance, PD-L1 binding partners are PD-1 and/or B7-1. In another instance, the PD-L2 binding antagonist is a molecule that inhibits the binding of PD-L2 to its ligand binding partners. In a specific instance, the PD-L2 binding ligand partner is PD-1. The antagonist may be an antibody, an antigen binding fragment thereof, an immunoadhesin, a fusion protein, or an oligopeptide. In some instances, the PD-1 binding antagonist is a small molecule, a nucleic acid, a polypeptide (e.g., antibody), a carbohydrate, a lipid, a metal, or a toxin.

In some instances, the PD-1 binding antagonist is an anti-PD-1 antibody (e.g., a human antibody, a humanized antibody, or a chimeric antibody), for example, as described below. In some instances, the anti-PD-1 antibody is MDX-1 106 (nivolumab), MK-3475 (pembrolizumab, Keytruda®), cemiplimab, dostarlimab, MEDI-0680 (AMP-514), PDR001, REGN2810, MGA-012, JNJ-63723283, BI 754091, or BGB-108. In other instances, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence)). In some instances, the PD-1 binding antagonist is AMP-224. Other examples of anti-PD-1 antibodies include, but are not limited to, MEDI-0680 (AMP-514; AstraZeneca), PDR001 (CAS Registry No. 1859072-53-9; Novartis), REGN2810 (LIBTAYO® or cemiplimab-rwlc; Regeneron), BGB-108 (BciGene), BGB-A317 (BeiGene), BI 754091, JS-001 (Shanghai Junshi), STI-A1110 (Sorrento), INCSHR-1210 (Incyte), PF-06801591 (Pfizer), TSR-042 (also known as ANB011; Tesaro/AnaptysBio), AM0001 (ARMO Biosciences), ENUM 244C8 (Enumeral Biomedical Holdings), or ENUM 388D4 (Enumeral Biomedical Holdings). In some instances, the PD-1 axis binding antagonist comprises tislelizumab (BGB-A317), BGB-108, STI-A1110, AM0001, BI 754091, sintilimab (IBI308), cetrclimab (JNJ-63723283), toripalimab (JS-001), camrelizumab (SHR-1210, INCSHR-1210, HR-301210), MEDI-0680 (AMP-514), MGA-012 (INCMGA 0012), nivolumab (BMS-936558, MDX1106, ONO-4538), spartalizumab (PDR001), pembrolizumab (MK-3475, SCH 900475, Keytruda®), PF-06801591, cemiplimab (REGN-2810, REGEN2810), dostarlimab (TSR-042, ANB011), FITC-YT-16 (PD-1 binding peptide), APL-501 or CBT-501 or genolimzumab (GB-226), AB-122, AK105, AMG 404, BCD-100, F520, HLX10, HX008, JTX-4014, LZM009, Sym021, PSB205, AMP-224 (fusion protein targeting PD-1), CX-188 (PD-1 probody), AGEN-2034, GLS-010, budigalimab (ABBV-181), AK-103, BAT-1306, CS-1003, AM-0001, TILT-123, BH-2922, BH-2941, BH-2950, ENUM-244C8, ENUM-388D4, HAB-21, H EISCOI 11-003, IKT-202, MCLA-134, MT-17000, PEGMP-7, PRS-332, RXI-762, STI-1110, VXM-10, XmAb-23104, AK-112, HLX-20, SSI-361, AT-16201, SNA-01, AB122, PD1-PIK, PF-06936308, RG-7769, CAB PD-1 Abs, AK-123, MEDI-3387, MEDI-5771, 4H1128Z-E27, REMD-288, SG-001, BY-24.3, CB-201, IBI-319, ONCR-177, Max-1, CS-4100, JBI-426, CCC-0701, or CCX-4503, or derivatives thereof.

In some instances, the PD-L1 binding antagonist is a small molecule that inhibits PD-1. In some instances, the PD-L1 binding antagonist is a small molecule that inhibits PD-L1. In some instances, the PD-L1 binding antagonist is a small molecule that inhibits PD-L1 and VISTA or PD-L1 and TIM3. In some instances, the PD-L1 binding antagonist is CA-170 (also known as AUPM-170). In some instances, the PD-L1 binding antagonist is an anti-PD-L1 antibody. In some instances, the anti-PD-L1 antibody can bind to a human PD-L1, for example a human PD-L1 as shown in UniProtKB/Swiss-Prot Accession No.Q9NZQ7.1, or a variant thereof. In some instances, the PD-L1 binding antagonist is a small molecule, a nucleic acid, a polypeptide (e.g., antibody), a carbohydrate, a lipid, a metal, or a toxin.

In some instances, the PD-L1 binding antagonist is an anti-PD-L1 antibody, for example, as described below. In some instances, the anti-PD-L1 antibody is capable of inhibiting the binding between PD-L1 and PD-1, and/or between PD-L1 and B7-1. In some instances, the anti-PD-L1 antibody is a monoclonal antibody. In some instances, the anti-PD-L1 antibody is an antibody fragment selected from a Fab, Fab′-SH, Fv, scFv, or F (ab′) 2 fragment. In some instances, the anti-PD-L1 antibody is a humanized antibody. In some instances, the anti-PD-L1 antibody is a human antibody. In some instances, the anti-PD-L1 antibody is selected from YW243.55.S70, MPDL3280A (atezolizumab), MDX-1 105, MEDI4736 (durvalumab), or MSB0010718C (avelumab). In some instances, the PD-L1 axis binding antagonist comprises atezolizumab, avelumab, durvalumab (imfinzi), BGB-A333, SHR-1316 (HTI-1088), CK-301, BMS-936559, envafolimab (KN035, ASC22), CS1001, MDX-1105 (BMS-936559), LY3300054, STI-A1014, FAZ053, CX-072, INCB086550, GNS-1480, CA-170, CK-301, M-7824, HTI-1088 (HTI-131, SHR-1316), MSB-2311, AK-106, AVA-004, BBI-801, CA-327, CBA-0710, CBT-502, FPT-155, IKT-201, IKT-703, 10-103, JS-003, KD-033, KY-1003, MCLA-145, MT-5050, SNA-02, BCD-135, APL-502 (CBT-402 or TQB2450), IMC-001, KD-045, INBRX-105, KN-046, IMC-2102, IMC-2101, KD-005, IMM-2502, 89Zr-CX-072, 89Zr-DFO-6E11, KY-1055, MEDI-1109, MT-5594, SL-279252, DSP-106, Gensci-047, REMD-290, N-809, PRS-344, FS-222, GEN-1046, BH-29xx, or FS-118, or a derivative thereof.

In some instances, the checkpoint inhibitor is an antagonist/inhibitor of CTLA4. In some instances, the checkpoint inhibitor is a small molecule antagonist of CTLA4. In some instances, the checkpoint inhibitor is an anti-CTLA4 antibody. CTLA4 is part of the CD28-B7 immunoglobulin superfamily of immune checkpoint molecules that acts to negatively regulate T cell activation, particularly CD28-dependent T cell responses. CTLA4 competes for binding to common ligands with CD28, such as CD80 (B7-1) and CD86 (B7-2), and binds to these ligands with higher affinity than CD28. Blocking CTLA4 activity (e.g., using an anti-CTLA4 antibody) is thought to enhance CD28-mediated costimulation (leading to increased T cell activation/priming), affect T cell development, and/or deplete Tregs (such as intratumoral Tregs). In some instances, the CTLA4 antagonist is a small molecule, a nucleic acid, a polypeptide (e.g., antibody), a carbohydrate, a lipid, a metal, or a toxin. In some instances, the CTLA-4 inhibitor comprises ipilimumab (IBI310, BMS-734016, MDX010, MDX-CTLA4, MEDI4736), tremelimumab (CP-675, CP-675,206), APL-509, AGEN1884, CS1002, AGEN1181, Abatacept (Orencia, BMS-188667, RG2077), BCD-145, ONC-392, ADU-1604, REGN4659, ADG116, KN044, KN046, or a derivative thereof.

In some instances, the anti-PD-1 antibody or antibody fragment is MDX-1106 (nivolumab), MK-3475 (pembrolizumab, Keytruda®), cemiplimab, dostarlimab, MEDI-0680 (AMP-514), PDR001, REGN2810, MGA-012, JNJ-63723283, BI 754091, BGB-108, BGB-A317, JS-001, STI-A1110, INCSHR-1210, PF-06801591, TSR-042, AM0001, ENUM 244C8, or ENUM 388D4. In some instances, the PD-1 binding antagonist is an anti-PD-1 immunoadhesin. In some instances, the anti-PD-1 immunoadhesin is AMP-224. In some instances, the anti-PD-L1 antibody or antibody fragment is YW243.55.S70, MPDL3280A (atezolizumab), MDX-1105, MEDI4736 (durvalumab), MSB0010718C (avelumab), LY3300054, STI-A1014, KN035, FAZ053, or CX-072.

In some instances, the immune checkpoint inhibitor comprises a LAG-3 inhibitor (e.g., an antibody, an antibody conjugate, or an antigen-binding fragment thereof). In some instances, the LAG-3 inhibitor comprises a small molecule, a nucleic acid, a polypeptide (e.g., an antibody), a carbohydrate, a lipid, a metal, or a toxin. In some instances, the LAG-3 inhibitor comprises a small molecule. In some instances, the LAG-3 inhibitor comprises a LAG-3 binding agent. In some instances, the LAG-3 inhibitor comprises an antibody, an antibody conjugate, or an antigen-binding fragment thereof. In some instances, the LAG-3 inhibitor comprises eftilagimod alpha (IMP321, IMP-321, EDDP-202, EOC-202), relatlimab (BMS-986016), GSK2831781 (IMP-731), LAG525 (IMP701), TSR-033, EVIP321 (soluble LAG-3 protein), BI 754111, IMP761, REGN3767, MK-4280, MGD-013, XmAb22841, INCAGN-2385, ENUM-006, AVA-017, AM-0003, iOnctura anti-LAG-3 antibody, Arcus Biosciences LAG-3 antibody, Sym022, a derivative thereof, or an antibody that competes with any of the preceding.

In some instances, the immune checkpoint inhibitor is monovalent and/or monospecific. In some instances, the immune checkpoint inhibitor is multivalent and/or multispecific.

In some instances, the immune checkpoint inhibitor may be administered in combination with an immunoregulatory molecule or a cytokine. An immunoregulatory profile is required to trigger an efficient immune response and balance the immunity in a subject. Examples of suitable immunoregulatory cytokines include, but are not limited to, interferons (e.g., IFNα, IFNβ and IFNγ), interleukins (e.g., IL-1, IL-2, IL-3, IL-4, IL-5, IL-6, IL-7, IL-8, IL-9, IL-10, IL-12 and IL-20), tumor necrosis factors (e.g., TNFa and TNFB), erythropoietin (EPO), FLT-3 ligand, glp10, TCA-3, MCP-1, MIF, MIP-la, MIP-1B, Rantes, macrophage colony stimulating factor (M-CSF), granulocyte colony stimulating factor (G-CSF), or granulocyte-macrophage colony stimulating factor (GM-CSF), as well as functional fragments thereof. In some instances, any immunomodulatory chemokine that binds to a chemokine receptor, i.e., a CXC, CC, C, or CX3C chemokine receptor, can be used in the context of the present disclosure. Examples of chemokines include, but are not limited to, MIP-3a (Lax), MIP-3B, Hcc-1, MPIF-1, MPIF-2, MCP-2, MCP-3, MCP-4, MCP-5, Eotaxin, Tarc, Elc, 1309, IL-8, GCP-2 Groa, Gro-B, Nap-2, Ena-78, Ip-10, MIG, I-Tac, SDF-1, or BCA-1 (Blc), as well as functional fragments thereof. In some instances, the immunoregulatory molecule is included with any of the treatments provided herein.

In some instances, the immune checkpoint inhibitor is a first line immune checkpoint inhibitor. In some instances, the immune checkpoint inhibitor is a second line immune checkpoint inhibitor. In some instances, an immune checkpoint inhibitor is administered in combination with one or more additional anti-cancer therapies or treatments.

In some instances, the methods of the disclosure further comprise treating a subject with the IO therapy. In some instances, an IO therapy is administered as a monotherapy. In some instances, the IO therapy comprises one or multiple IO agents.

### C. Anti-Cancer Therapies

Certain aspects of the present disclosure provide for anti-cancer therapies.

In some instances, the anti-cancer therapy comprises a kinase inhibitor. In some instances, the methods provided herein comprise administering to the subject a kinase inhibitor, e.g., in combination with another anti-cancer therapy such as an immune checkpoint inhibitor. Examples of kinase inhibitors include those that target one or more receptor tyrosine kinases, e.g., BCR-ABL, B-Raf, EGFR, HER-2/ErbB2, IGF-IR, PDGFR-a, PDGFR-β, cKit, Flt-4, Flt3, FGFR1, FGFR3, FGFR4, CSFIR, c-Met, RON, c-Ret, or ALK; one or more cytoplasmic tyrosine kinases, e.g., c-SRC, c-YES, Abl, or JAK-2; one or more serine/threonine kinases, e.g., ATM, Aurora A & B, CDKs, mTOR, PKCi, PLKs, b-Raf, S6K, or STK11/LKB1; or one or more lipid kinases, e.g., PI3K or SKI. Small molecule kinase inhibitors include PHA-739358, nilotinib, dasatinib, PD166326, NSC 743411, lapatinib (GW-572016), canertinib (CI-1033), semaxinib (SU5416), vatalanib (PTK787/ZK222584), sutent (SU1 1248), sorafenib (BAY 43-9006), or leflunomide (SU101). Additional non-limiting examples of tyrosine kinase inhibitors include imatinib (Gleevec/Glivec) and gefitinib (Iressa).

In some instances, the anti-cancer therapy comprises an anti-angiogenic agent. In some instances, the methods provided herein comprise administering to the subject an anti-angiogenic agent, e.g., in combination with another anti-cancer therapy such as an immune checkpoint inhibitor. Angiogenesis inhibitors prevent the extensive growth of blood vessels (angiogenesis) that tumors require to survive. Non-limiting examples of angiogenesis-mediating molecules or angiogenesis inhibitors which may be used in the methods of the present disclosure include soluble VEGF (for example: VEGF isoforms, e.g., VEGF121 and VEGF165; VEGF receptors, e.g., VEGFR1, VEGFR2; and co-receptors, e.g., Neuropilin-1 and Neuropilin-2), NRP-1, angiopoietin 2, TSP-1 and TSP-2, angiostatin and related molecules, endostatin, vasostatin, calreticulin, platelet factor-4, TIMP and CDAI, Meth-1 and Meth-2, IFNα, IFN-β and IFN-γ, CXCL10, IL-4, IL-12 and IL-18, prothrombin (kringle domain-2), antithrombin III fragment, prolactin, VEGI, SPARC, osteopontin, maspin, canstatin, proliferin-related protein, restin and drugs such as bevacizumab, itraconazole, carboxyamidotriazole, TNP-470, CM101, IFN-α platelet factor-4, suramin, SU5416, thrombospondin, VEGFR antagonists, angiostatic steroids and heparin, cartilage-derived angiogenesis inhibitory factor, matrix metalloproteinase inhibitors, 2-methoxyestradiol, tecogalan, tetrathiomolybdate, thalidomide, thrombospondin, prolactina v β3 inhibitors, linomide, or tasquinimod. In some instances, known therapeutic candidates that may be used according to the methods of the disclosure include naturally occurring angiogenic inhibitors, including without limitation, angiostatin, endostatin, or platelet factor-4. In another instance, therapeutic candidates that may be used according to the methods of the disclosure include, without limitation, specific inhibitors of endothelial cell growth, such as TNP-470, thalidomide, and interleukin-12. Still other anti-angiogenic agents that may be used according to the methods of the disclosure include those that neutralize angiogenic molecules, including without limitation, antibodies to fibroblast growth factor, antibodies to vascular endothelial growth factor, antibodies to platelet derived growth factor, or antibodies or other types of inhibitors of the receptors of EGF, VEGF or PDGF. In some instances, anti-angiogenic agents that may be used according to the methods of the disclosure include, without limitation, suramin and its analogs, and tecogalan. In other instances, anti-angiogenic agents that may be used according to the methods of the disclosure include, without limitation, agents that neutralize receptors for angiogenic factors or agents that interfere with vascular basement membrane and extracellular matrix, including, without limitation, metalloprotease inhibitors and angiostatic steroids. Another group of anti-angiogenic compounds that may be used according to the methods of the disclosure includes, without limitation, anti-adhesion molecules, such as antibodies to integrin alpha v beta 3. Still other anti-angiogenic compounds or compositions that may be used according to the methods of the disclosure include, without limitation, kinase inhibitors, thalidomide, itraconazole, carboxyamidotriazole, CM101, IFN-α, IL-12, SU5416, thrombospondin, cartilage-derived angiogenesis inhibitory factor, 2-methoxyestradiol, tetrathiomolybdate, thrombospondin, prolactin, and linomide. In one particular instance, the anti-angiogenic compound that may be used according to the methods of the disclosure is an antibody to VEGF, such as Avastin®/bevacizumab (Genentech).

In some instances, the anti-cancer therapy comprises an anti-DNA repair therapy. In some instances, the methods provided herein comprise administering to the subject an anti-DNA repair therapy, e.g., in combination with another anti-cancer therapy such as an immune checkpoint inhibitor. In some instances, the anti-DNA repair therapy is a PARP inhibitor (e.g., talazoparib, rucaparib, olaparib), a RAD51 inhibitor (e.g., RI-1), or an inhibitor of a DNA damage response kinase, e.g., CHCK1 (e.g., AZD7762), ATM (e.g., KU-55933, KU-60019, NU7026, or VE-821), and ATR (e.g., NU7026).

In some instances, the anti-cancer therapy comprises a radiosensitizer. In some instances, the methods provided herein comprise administering to the subject a radiosensitizer, e.g., in combination with another anti-cancer therapy such as an immune checkpoint inhibitor. Exemplary radiosensitizers include hypoxia radiosensitizers such as misonidazole, metronidazole, and trans-sodium crocetinate, a compound that helps to increase the diffusion of oxygen into hypoxic tumor tissue. The radiosensitizer can also be a DNA damage response inhibitor interfering with base excision repair (BER), nucleotide excision repair (NER), mismatch repair (MMR), recombinational repair comprising homologous recombination (HR) and non-homologous end-joining (NHEJ), and direct repair mechanisms. Single strand break (SSB) repair mechanisms include BER, NER, or MMR pathways, while double stranded break (DSB) repair mechanisms consist of HR and NHEJ pathways. Radiation causes DNA breaks that, if not repaired, are lethal. SSBs are repaired through a combination of BER, NER and MMR mechanisms using the intact DNA strand as a template. The predominant pathway of SSB repair is BER, utilizing a family of related enzymes termed poly-(ADP-ribose) polymerases (PARP). Thus, the radiosensitizer can include DNA damage response inhibitors such as PARP inhibitors.

In some instances, the anti-cancer therapy comprises an anti-inflammatory agent. In some instances, the methods provided herein comprise administering to the subject an anti-inflammatory agent, e.g., in combination with another anti-cancer therapy such as an immune checkpoint inhibitor. In some instances, the anti-inflammatory agent is an agent that blocks, inhibits, or reduces inflammation or signaling from an inflammatory signaling pathway In some instances, the anti-inflammatory agent inhibits or reduces the activity of one or more of any of the following: IL-1, IL-2, IL-3, IL-4, IL-5, IL-6, IL-7, IL-8, IL-9, IL-10, IL-12, IL-13, IL-15, IL-18, IL-23; interferons (IFNs), e.g., IFNα, IFNβ, IFNγ, IFN-γ inducing factor (IGIF); transforming growth factor-β (TGF-β); transforming growth factor-α (TGF-α); tumor necrosis factors, e.g., TNF-α, TNF-β, TNF-RI, TNF-RII; CD23; CD30; CD40L; EGF; G-CSF; GDNF; PDGF-BB; RANTES/CCL5; IKK; NF-κB; TLR2; TLR3; TLR4; TL5; TLR6; TLR7; TLR8; TLR8; TLR9; and/or any cognate receptors thereof. In some instances, the anti-inflammatory agent is an IL-1 or IL-1 receptor antagonist, such as anakinra (Kineret®), rilonacept, or canakinumab. In some instances, the anti-inflammatory agent is an IL-6 or IL-6 receptor antagonist, e.g., an anti-IL-6 antibody or an anti-IL-6 receptor antibody, such as tocilizumab (ACTEMRA®), olokizumab, clazakizumab, sarilumab, sirukumab, siltuximab, or ALX-0061. In some instances, the anti-inflammatory agent is a TNF-α antagonist, e.g., an anti-TNFα antibody, such as infliximab (Remicade®), golimumab (Simponi®), adalimumab (Humira®), certolizumab pegol (Cimzia®) or etanercept. In some instances, the anti-inflammatory agent is a corticosteroid. Exemplary corticosteroids include, but are not limited to, cortisone (hydrocortisone, hydrocortisone sodium phosphate, hydrocortisone sodium succinate, Ala-Cort®, Hydrocort Acetate®, hydrocortone phosphate Lanacort®, Solu-Cortef®), decadron (dexamethasone, dexamethasone acetate, dexamethasone sodium phosphate, Dexasone®, Diodex®, Hexadrol®, Maxidex®), methylprednisolone (6-methylprednisolone, methylprednisolone acetate, methylprednisolone sodium succinate, Duralone®, Medralone®, Medrol®, M-Prednisol®, Solu-Medrol®), prednisolone (Delta-Cortef®, ORAPRED®, Pediapred®, Prezone®), and prednisone (Deltasone®, Liquid Pred®, Meticorten®, Orasone®), and bisphosphonates (e.g., pamidronate (Aredia®), and zoledronic acid (Zometac®).

In some instances, the anti-cancer therapy comprises an anti-hormonal agent. In some instances, the methods provided herein comprise administering to the subject an anti-hormonal agent, e.g., in combination with another anti-cancer therapy such as an immune checkpoint inhibitor. Anti-hormonal agents are agents that act to regulate or inhibit hormone action on tumors. Examples of anti-hormonal agents include anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON® toremifene; aromatase inhibitors that inhibit the enzyme aromatase, which regulates estrogen production in the adrenal glands, such as, for example, 4 (5)-imidazoles, aminoglutethimide, MEGACER megestrol acetate, AROMASIN® exemestane, formestanie, fadrozole, RIVISOR® vorozole, FEMARA® letrozole, and ARIMIDEX® (anastrozole); anti—androgens such as flutamide, nilutamide, bicalutamide, leuprolide, and goserelin; troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids or derivatives of any of the above.

In some instances, the anti-cancer therapy comprises an antimetabolite chemotherapeutic agent. In some instances, the methods provided herein comprise administering to the subject an antimetabolite chemotherapeutic agent, e.g., in combination with another anti-cancer therapy such as an immune checkpoint inhibitor. Antimetabolite chemotherapeutic agents are agents that are structurally similar to a metabolite, but cannot be used by the body in a productive manner. Many antimetabolite chemotherapeutic agents interfere with the production of RNA or DNA. Examples of antimetabolite chemotherapeutic agents include gemcitabine (GEMZAR®), 5-fluorouracil (5-FU), capecitabine (XELODA™), 6-mercaptopurine, methotrexate, 6-thioguanine, pemetrexed, raltitrexed, arabinosylcytosine ARA-C cytarabine (CYTOSAR-U®), dacarbazine (DTIC-DOMED), azocytosine, deoxycytosine, pyridmidene, fludarabine (FLUDARA®), cladrabine, and 2-deoxy-D-glucose. In some instances, an antimetabolite chemotherapeutic agent is gemcitabine. Gemcitabine HCL is sold by Eli Lilly under the trademark GEMZAR®.

In some instances, the anti-cancer therapy comprises a platinum-based chemotherapeutic agent. In some instances, the methods provided herein comprise administering to the subject a platinum-based chemotherapeutic agent, e.g., in combination with another anti-cancer therapy such as an immune checkpoint inhibitor. Platinum-based chemotherapeutic agents are chemotherapeutic agents that comprise an organic compound containing platinum as an integral part of the molecule. In some instances, a chemotherapeutic agent is a platinum agent. In some such instances, the platinum agent is selected from cisplatin, carboplatin, oxaliplatin, nedaplatin, triplatin tetranitrate, phenanthriplatin, picoplatin, or satraplatin.

In some instances, the anti-cancer therapy comprises a cancer immunotherapy, such as a cancer vaccine, cell-based therapy, T cell receptor (TCR)-based therapy, adjuvant immunotherapy, cytokine immunotherapy, and oncolytic virus therapy. In some instances, the methods provided herein comprise administering to the subject a cancer immunotherapy, such as a cancer vaccine, cell-based therapy, T cell receptor (TCR)-based therapy, adjuvant immunotherapy, cytokine immunotherapy, and oncolytic virus therapy, e.g., in combination with another anti-cancer therapy such as an immune checkpoint inhibitor. In some instances, the cancer immunotherapy comprises a small molecule, nucleic acid, polypeptide, carbohydrate, toxin, cell-based agent, or cell-binding agent. Examples of cancer immunotherapies are described in greater detail herein but are not intended to be limiting. In some instances, the cancer immunotherapy activates one or more aspects of the immune system to attack a cell (e.g., a tumor cell) that expresses a neoantigen, e.g., a neoantigen expressed by a cancer of the disclosure. The cancer immunotherapies of the present disclosure are contemplated for use as monotherapies, or in combination approaches comprising two or more in any combination or number, subject to medical judgement. Any of the cancer immunotherapies (optionally as monotherapies or in combination with another cancer immunotherapy or other therapeutic agent described herein) may find use in any of the methods described herein.

In some instances, the cancer immunotherapy comprises a cancer vaccine. A range of cancer vaccines have been tested that employ different approaches to promoting an immune response against a cancer (see, e.g., Emens L A, Expert Opin Emerg Drugs 13 (2): 295-308 (2008) and US20190367613). Approaches have been designed to enhance the response of B cells, T cells, or professional antigen-presenting cells against tumors. Exemplary types of cancer vaccines include, but are not limited to, DNA-based vaccines, RNA-based vaccines, virus transduced vaccines, peptide-based vaccines, dendritic cell vaccines, oncolytic viruses, whole tumor cell vaccines, tumor antigen vaccines, etc. In some instances, the cancer vaccine can be prophylactic or therapeutic. In some instances, the cancer vaccine is formulated as a peptide-based vaccine, a nucleic acid-based vaccine, an antibody based vaccine, or a cell based vaccine.

For example, a vaccine composition can include naked cDNA in cationic lipid formulations; lipopeptides (e.g., Vitiello, A. ct ah, J. Clin. Invest. 95:341, 1995), naked cDNA or peptides, encapsulated e.g., in poly (DL-lactide-co-glycolide) (“PLG”) microspheres (see, e.g., Eldridge, ct ah, Molec. Immunol. 28:287-294, 1991: Alonso et al, Vaccine 12:299-306, 1994; Jones et al, Vaccine 13:675-681, 1995); peptide composition contained in immune stimulating complexes (ISCOMS) (e.g., Takahashi et al, Nature 344:873-875, 1990; Hu et al, Clin. Exp. Immunol. 113:235-243, 1998); or multiple antigen peptide systems (MAPs) (sce e.g., Tam, J. P., Proc. Natl Acad. Sci. U.S.A. 85:5409-5413, 1988; Tam, J.P., J. Immunol. Methods 196:17-32, 1996). In some instances, a cancer vaccine is formulated as a peptide-based vaccine, or nucleic acid based vaccine in which the nucleic acid encodes the polypeptides. In some instances, a cancer vaccine is formulated as an antibody-based vaccine. In some instances, a cancer vaccine is formulated as a cell based vaccine. In some instances, the cancer vaccine is a peptide cancer vaccine, which in some instances is a personalized peptide vaccine. In some instances, the cancer vaccine is a multivalent long peptide, a multiple peptide, a peptide mixture, a hybrid peptide, or a peptide pulsed dendritic cell vaccine (see, e.g., Yamada et al, Cancer Sci, 104:14-21), 2013). In some instances, such cancer vaccines augment the anti-cancer response.

In some instances, the cancer vaccine comprises a polynucleotide that encodes a neoantigen, e.g., a neoantigen expressed by a cancer of the disclosure. In some instances, the cancer vaccine comprises DNA or RNA that encodes a neoantigen. In some instances, the cancer vaccine comprises a polynucleotide that encodes a neoantigen. In some instances, the cancer vaccine further comprises one or more additional antigens, neoantigens, or other sequences that promote antigen presentation and/or an immune response. In some instances, the polynucleotide is complexed with one or more additional agents, such as a liposome or lipoplex. In some instances, the polynucleotide(s) are taken up and translated by antigen presenting cells (APCs), which then present the neoantigen(s) via MHC class I on the APC cell surface.

In some instances, the cancer vaccine is selected from sipuleucel-T (Provenge®, Dendrcon/Valeant Pharmaceuticals), which has been approved for treatment of asymptomatic, or minimally symptomatic metastatic castrate-resistant (hormone-refractory) prostate cancer; and talimogene laherparepvec (Imlygic®, BioVex/Amgen, previously known as T-VEC), a genetically modified oncolytic viral therapy approved for treatment of unresectable cutaneous, subcutaneous and nodal lesions in melanoma. In some instances, the cancer vaccine is selected from an oncolytic viral therapy such as pexastimogene devacirepvec (PexaVec/JX-594, SillaJen/formerly Jennerex Biotherapeutics), a thymidine kinase-(TK-) deficient vaccinia virus engineered to express GM-CSF, for hepatocellular carcinoma (NCT02562755) and melanoma (NCT00429312); pelareorep (Reolysin®, Oncolytics Biotech), a variant of respiratory enteric orphan virus (reovirus) which does not replicate in cells that are not RAS-activated, in numerous cancers, including colorectal cancer (NCT01622543), prostate cancer (NCT01619813), head and neck squamous cell cancer (NCT01166542), pancreatic adenocarcinoma (NCT00998322), and non-small cell lung cancer (NSCLC) (NCT 00861627); enadenotucirev (NG-348, PsiOxus, formerly known as ColoAdl), an adenovirus engineered to express a full length CD80 and an antibody fragment specific for the T-cell receptor CD3 protein, in ovarian cancer (NCT02028117), metastatic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-CSF, in melanoma (NCT03003676), and peritoneal disease, colorectal cancer or ovarian cancer (NCT02963831); GL-ONCI (GLV-1h68/GLV-1h153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260), fallopian tube cancer, ovarian cancer (NCT 02759588); or CG0070 (Cold Genesys), an adenovirus engineered to express GM-CSF in bladder cancer (NCT02365818); anti-gp100; STINGVAX; GVAX; DCVaxL; and DNX-2401. In some instances, the cancer vaccine is selected from JX-929 (SillaJen/formerly Jennerex Biotherapeutics), a TK—and vaccinia growth factor-deficient vaccinia virus engineered to express cytosine deaminase, which is able to convert the prodrug 5-fluorocytosine to the cytotoxic drug 5-fluorouracil; TGO1 and TG02 (Targovax/formerly Oncos), peptide-based immunotherapy agents targeted for difficult-to-treat RAS mutations; and TILT-123 (TILT Biotherapeutics), an engineered adenovirus designated: Ad5/3-E2F-delta24-hTNFα-IRES-hIL20; and VSV-GP (Vira Therapeutics) a vesicular stomatitis virus (VSV) engineered to express the glycoprotein (GP) of lymphocytic choriomeningitis virus (LCMV), which can be further engineered to express antigens designed to raise an antigen-specific CD8+ T cell response. In some instances, the cancer vaccine comprises a vector-based tumor antigen vaccine. Vector-based tumor antigen vaccines can be used as a way to provide a steady supply of antigens to stimulate an anti-tumor immune response. In some instances, vectors encoding for tumor antigens are injected into a subject (possibly with pro-inflammatory or other attractants such as GM-CSF), taken up by cells in vivo to make the specific antigens, which then provoke the desired immune response. In some instances, vectors may be used to deliver more than one tumor antigen at a time, to increase the immune response. In addition, recombinant virus, bacteria or yeast vectors can trigger their own immune responses, which may also enhance the overall immune response.

In some instances, the cancer vaccine comprises a DNA-based vaccine. In some instances, DNA-based vaccines can be employed to stimulate an anti-tumor response. The ability of directly injected DNA that encodes an antigenic protein, to elicit a protective immune response has been demonstrated in numerous experimental systems. Vaccination through directly injecting DNA that encodes an antigenic protein, to elicit a protective immune response often produces both cell-mediated and humoral responses. Moreover, reproducible immune responses to DNA encoding various antigens have been reported in mice that last essentially for the lifetime of the animal (see, e.g., Yankauckas et al. (1993) DNA Cell Biol., 12:771-776). In some instances, plasmid (or other vector) DNA that includes a sequence encoding a protein operably linked to regulatory elements required for gene expression is administered to subjects (e.g. human patients, non-human mammals, etc.). In some instances, the cells of the subject take up the administered DNA and the coding sequence is expressed. In some instances, the antigen so produced becomes a target against which an immune response is directed.

In some instances, the cancer vaccine comprises an RNA-based vaccine. In some instances, RNA-based vaccines can be employed to stimulate an anti-tumor response. In some instances, RNA-based vaccines comprise a self-replicating RNA molecule. In some instances, the self-replicating RNA molecule may be an alphavirus-derived RNA replicon. Self-replicating RNA (or “SAM”) molecules are well known in the art and can be produced by using replication elements derived from, e.g., alphaviruses, and substituting the structural viral proteins with a nucleotide sequence encoding a protein of interest. A self-replicating RNA molecule is typically a +-strand molecule which can be directly translated after delivery to a cell, and this translation provides a RNA-dependent RNA polymerase which then produces both antisense and sense transcripts from the delivered RNA. Thus, the delivered RNA leads to the production of multiple daughter RNAs. These daughter RNAs, as well as collinear subgenomic transcripts, may be translated themselves to provide in situ expression of an encoded polypeptide, or may be transcribed to provide further transcripts with the same sense as the delivered RNA which are translated to provide in situ expression of the antigen.

In some instances, the cancer immunotherapy comprises a cell-based therapy. In some instances, the cancer immunotherapy comprises a T cell-based therapy. In some instances, the cancer immunotherapy comprises an adoptive therapy, e.g., an adoptive T cell-based therapy. In some instances, the T cells are autologous or allogeneic to the recipient. In some instances, the T cells are CD8+ T cells. In some instances, the T cells are CD4+ T cells. Adoptive immunotherapy refers to a therapeutic approach for treating cancer or infectious diseases in which immune cells are administered to a host with the aim that the cells mediate either directly or indirectly specific immunity to (i.e., mount an immune response directed against) cancer cells. In some instances, the immune response results in inhibition of tumor and/or metastatic cell growth and/or proliferation, and in related instances, results in neoplastic cell death and/or resorption. The immune cells can be derived from a different organism/host (exogenous immune cells) or can be cells obtained from the subject organism (autologous immune cells). In some instances, the immune cells (e.g., autologous or allogeneic T cells (e.g., regulatory T cells, CD4+ T cells, CD8+ T cells, or gamma-delta T cells), NK cells, invariant NK cells, or NKT cells) can be genetically engineered to express antigen receptors such as engineered TCRs and/or chimeric antigen receptors (CARs). For example, the host cells (e.g., autologous or allogeneic T-cells) are modified to express a T cell receptor (TCR) having antigenic specificity for a cancer antigen. In some instances, NK cells are engineered to express a TCR. The NK cells may be further engineered to express a CAR. Multiple CARs and/or TCRs, such as to different antigens, may be added to a single cell type, such as T cells or NK cells. In some instances, the cells comprise one or more nucleic acids/expression constructs/vectors introduced via genetic engineering that encode one or more antigen receptors, and genetically engineered products of such nucleic acids. In some instances, the nucleic acids are heterologous, i.e., normally not present in a cell or sample obtained from the cell, such as one obtained from another organism or cell, which for example, is not ordinarily found in the cell being engineered and/or an organism from which such cell is derived. In some instances, the nucleic acids are not naturally occurring, such as a nucleic acid not found in nature (e.g. chimeric). In some instances, a population of immune cells can be obtained from a subject in need of therapy or suffering from a disease associated with reduced immune cell activity. Thus, the cells will be autologous to the subject in need of therapy. In some instances, a population of immune cells can be obtained from a donor, such as a histocompatibility-matched donor. In some instances, the immune cell population can be harvested from the peripheral blood, cord blood, bone marrow, spleen, or any other organ/tissue in which immune cells reside in said subject or donor. In some instances, the immune cells can be isolated from a pool of subjects and/or donors, such as from pooled cord blood. In some instances, when the population of immune cells is obtained from a donor distinct from the subject, the donor may be allogeneic, provided the cells obtained are subject-compatible, in that they can be introduced into the subject. In some instances, allogeneic donor cells may or may not be human-leukocyte-antigen (HLA)-compatible. In some instances, to be rendered subject-compatible, allogeneic cells can be treated to reduce immunogenicity.

In some instances, the cell-based therapy comprises a T cell-based therapy, such as autologous cells, e.g., tumor-infiltrating lymphocytes (TILs); T cells activated ex-vivo using autologous DCs, lymphocytes, artificial antigen-presenting cells (APCs) or beads coated with T cell ligands and activating antibodies, or cells isolated by virtue of capturing target cell membrane; allogeneic cells naturally expressing anti-host tumor T cell receptor (TCR); and non-tumor-specific autologous or allogeneic cells genetically reprogrammed or “redirected” to express tumor-reactive TCR or chimeric TCR molecules displaying antibody-like tumor recognition capacity known as “T-bodies”. Several approaches for the isolation, derivation, engineering or modification, activation, and expansion of functional anti-tumor effector cells have been described in the last two decades and may be used according to any of the methods provided herein. In some instances, the T cells are derived from the blood, bone marrow, lymph, umbilical cord, or lymphoid organs. In some instances, the cells are human cells. In some instances, the cells are primary cells, such as those isolated directly from a subject and/or isolated from a subject and frozen. In some instances, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. In some instances, the cells may be allogeneic and/or autologous. In some instances, such as for off-the-shelf technologies, the cells are pluripotent and/or multipotent, such as stem cells, such as induced pluripotent stem cells (iPSCs).

In some instances, the T cell-based therapy comprises a chimeric antigen receptor (CAR)-T cell-based therapy. This approach involves engineering a CAR that specifically binds to an antigen of interest and comprises one or more intracellular signaling domains for T cell activation. The CAR is then expressed on the surface of engineered T cells (CAR-T) and administered to a subject, leading to a T-cell-specific immune response against cancer cells expressing the antigen.

In some instances, the T cell-based therapy comprises T cells expressing a recombinant T cell receptor (TCR). This approach involves identifying a TCR that specifically binds to an antigen of interest, which is then used to replace the endogenous or native TCR on the surface of engineered T cells that are administered to a subject, leading to a T-cell-specific immune response against cancer cells expressing the antigen.

In some instances, the T cell-based therapy comprises tumor-infiltrating lymphocytes (TILs). For example, TILs can be isolated from a tumor or cancer of the present disclosure, then isolated and expanded in vitro. Some or all of these TILs may specifically recognize an antigen expressed by the tumor or cancer of the present disclosure. In some instances, the TILs are exposed to one or more neoantigens, e.g., a neoantigen, in vitro after isolation. TILs are then administered to the subject (optionally in combination with one or more cytokines or other immune-stimulating substances).

In some instances, the cell-based therapy comprises a natural killer (NK) cell-based therapy. Natural killer (NK) cells are a subpopulation of lymphocytes that have spontaneous cytotoxicity against a variety of tumor cells, virus-infected cells, and some normal cells in the bone marrow and thymus. NK cells are critical effectors of the early innate immune response toward transformed and virus-infected cells. NK cells can be detected by specific surface markers, such as CD16, CD56, and CD8 in humans. NK cells do not express T-cell antigen receptors, the pan T marker CD3, or surface immunoglobulin B cell receptors. In some instances, NK cells are derived from human peripheral blood mononuclear cells (PBMC), unstimulated leukapheresis products (PBSC), human embryonic stem cells (hESCs), induced pluripotent stem cells (iPSCs), bone marrow, or umbilical cord blood by methods well known in the art.

In some instances, the cell-based therapy comprises a dendritic cell (DC)-based therapy, e.g., a dendritic cell vaccine. In some instances, the DC vaccine comprises antigen-presenting cells that are able to induce specific T cell immunity, which are harvested from the subject or from a donor. In some instances, the DC vaccine can then be exposed in vitro to a peptide antigen, for which T cells are to be generated in the subject. In some instances, dendritic cells loaded with the antigen are then injected back into the subject. In some instances, immunization may be repeated multiple times if desired. Methods for harvesting, expanding, and administering dendritic cells are known in the art; see, e.g., WO2019178081. Dendritic cell vaccines (such as Sipuleucel-T, also known as APC8015 and PROVENGER) are vaccines that involve administration of dendritic cells that act as APCs to present one or more cancer-specific antigens to the subject's immune system. In some instances, the dendritic cells are autologous or allogeneic to the recipient.

In some instances, the cancer immunotherapy comprises a TCR-based therapy. In some instances, the cancer immunotherapy comprises administration of one or more TCRs or TCR-based therapeutics that specifically bind an antigen expressed by a cancer of the present disclosure. In some instances, the TCR-based therapeutic may further include a moiety that binds an immune cell (e.g., a T cell), such as an antibody or antibody fragment that specifically binds a T cell surface protein or receptor (e.g., an anti-CD3 antibody or antibody fragment).

In some instances, the immunotherapy comprises adjuvant immunotherapy. Adjuvant immunotherapy comprises the use of one or more agents that activate components of the innate immune system, e.g., HILTONOL® (imiquimod), which targets the TLR7 pathway.

In some instances, the immunotherapy comprises cytokine immunotherapy. Cytokine immunotherapy comprises the use of one or more cytokines that activate components of the immune system. Examples include, but are not limited to, aldesleukin (PROLEUKIN®; interleukin-2), interferon alfa-2a (ROFERON®-A), interferon alfa-2b (INTRON®-A), and peginterferon alfa-2b (PEGINTRON®).

In some instances, the immunotherapy comprises oncolytic virus therapy. Oncolytic virus therapy uses genetically modified viruses to replicate in and kill cancer cells, leading to the release of antigens that stimulate an immune response. In some instances, replication-competent oncolytic viruses expressing a tumor antigen comprise any naturally occurring (e.g., from a “field source”) or modified replication-competent oncolytic virus. In some instances, the oncolytic virus, in addition to expressing a tumor antigen, may be modified to increase selectivity of the virus for cancer cells. In some instances, replication-competent oncolytic viruses include, but are not limited to, oncolytic viruses that are a member in the family of myoviridae, siphoviridae, podpviridae, teciviridae, corticoviridae, plasmaviridae, lipothrixviridae, fuselloviridae, poxyiridae, iridoviridae, phycodnaviridae, baculoviridae, herpesviridae, adnoviridae, papovaviridae, polydnaviridae, inoviridae, microviridae, geminiviridae, circoviridae, parvoviridae, hepadnaviridae, retroviridae, cyctoviridae, reoviridae, birnaviridae, paramyxoviridae, rhabdoviridae, filoviridae, orthomyxoviridae, bunyaviridae, arenaviridae, Leviviridae, picornaviridae, sequiviridae, comoviridae, potyviridae, caliciviridae, astroviridae, nodaviridae, tetraviridae, tombusviridae, coronaviridae, glaviviridae, togaviridae, and barnaviridae. In some instances, replication-competent oncolytic viruses include adenovirus, retrovirus, reovirus, rhabdovirus, Newcastle Disease virus (NDV), polyoma virus, vaccinia virus (VacV), herpes simplex virus, picornavirus, coxsackie virus and parvovirus. In some instances, a replicative oncolytic vaccinia virus expressing a tumor antigen may be engineered to lack one or more functional genes in order to increase the cancer selectivity of the virus. In some instances, an oncolytic vaccinia virus is engineered to lack thymidine kinase (TK) activity. In some instances, the oncolytic vaccinia virus may be engineered to lack vaccinia virus growth factor (VGF). In some instances, an oncolytic vaccinia virus may be engineered to lack both VGF and TK activity. In some instances, an oncolytic vaccinia virus may be engineered to lack one or more genes involved in evading host interferon (IFN) response such as E3L, K3L, B18R, or B8R. In some instances, a replicative oncolytic vaccinia virus is a Western Reserve, Copenhagen, Lister or Wyeth strain and lacks a functional TK gene. In some instances, the oncolytic vaccinia virus is a Western Reserve, Copenhagen, Lister or Wyeth strain lacking a functional B18R and/or B8R gene. In some instances, a replicative oncolytic vaccinia virus expressing a tumor antigen may be locally or systemically administered to a subject, e.g. via intratumoral, intraperitoneal, intravenous, intra-arterial, intramuscular, intradermal, intracranial, subcutaneous, or intranasal administration.

In some instances, the anti-cancer therapy comprises a nucleic acid molecule, such as a dsRNA, an siRNA, or an shRNA. In some instances, the methods provided herein comprise administering to the subject a nucleic acid molecule, such as a dsRNA, an siRNA, or an shRNA, e.g., in combination with another anti-cancer therapy. As is known in the art, dsRNAs having a duplex structure are effective at inducing RNA interference (RNAi). In some instances, the anti-cancer therapy comprises a small interfering RNA molecule (siRNA). dsRNAs and siRNAs can be used to silence gene expression in mammalian cells (e.g., human cells). In some instances, a dsRNA of the disclosure comprises any of between about 5 and about 10 base pairs, between about 10 and about 12 base pairs, between about 12 and about 15 base pairs, between about 15 and about 20 base pairs, between about 20 and 23 base pairs, between about 23 and about 25 base pairs, between about 25 and about 27 base pairs, or between about 27 and about 30 base pairs. As is known in the art, siRNAs are small dsRNAs that optionally include overhangs. In some instances, the duplex region of an siRNA is between about 18 and 25 nucleotides, e.g., any of 18, 19, 20, 21, 22, 23, 24, or 25 nucleotides. siRNAs may also include short hairpin RNAs (shRNAs), e.g., with approximately 29-base-pair stems and 2-nucleotide 3′ overhangs. Methods for designing, optimizing, producing, and using dsRNAs, siRNAs, or shRNAs, are known in the art.

### IV. Systems

Also disclosed herein are systems designed to implement any of the disclosed methods for determining risk scores that predict the likelihood of response to a selected treatment in a subject. The systems may comprise, e.g., one or more processors, and a memory unit communicatively coupled to the one or more processors and configured to store instructions that, when executed by the one or more processors, cause the system to: receive genomic data comprising aneuploidy status for one or more subgenomic intervals in each of a plurality of patients exhibiting the disease who have been treated using the selected treatment; perform a statistical analysis of the genomic data for the plurality of patients to identify one or more subgenomic intervals for which aneuploidy is correlated with a patient survival metric for the selected treatment; and train a machine learning model configured to receive genomic data comprising aneuploidy status for the one or more identified subgenomic intervals in a subject and output a risk score for the subject, wherein the risk score predicts a response to the selected treatment for the subject.

In some instances, the disclosed systems may further comprise a sequencer, e.g., a next generation sequencer (also referred to as a massively parallel sequencer). Examples of next generation (or massively parallel) sequencing platforms include, but are not limited to, Roche/454's Genome Sequencer (GS) FLX system, Illumina/Solexa's Genome Analyzer (GA), Illumina's HiSeq® 2500, HiSeq® 3000, HiSeq® 4000 and NovaSeq® 6000 sequencing systems, Life/APG's Support Oligonucleotide Ligation Detection (SOLID) system, Polonator's G.007 system, Helicos BioSciences' HeliScope Gene Sequencing system, ThermoFisher Scientific's Ion Torrent Genexus system, or Pacific Biosciences' PacBio® RS system.

In some instances, the disclosed systems may be used for determining risk scores that predict the likelihood of a response to a selected treatment in a subject based on genomic data derived from any of a variety of samples as described herein (e.g., a tissue sample, biopsy sample, hematological sample, or liquid biopsy sample derived from the subject).

In some instances, the plurality of gene loci and/or subgenomic intervals for which sequencing data is processed to determine risk scores that predict the likelihood of a response to a selected treatment in a subject may comprise at least 1, 2, 3, 4, 5, 6, 7, 8, 9, 10, 20, 30, 40, 50, 60, 70, 80, 90, 100, or more than 100 gene loci and/or subgenomic intervals.

In some instance, the nucleic acid sequence data is acquired using a next generation sequencing technique (also referred to as a massively parallel sequencing technique) having a read-length of less than 400 bases, less than 300 bases, less than 200 bases, less than 150 bases, less than 100 bases, less than 90 bases, less than 80 bases, less than 70 bases, less than 60 bases, less than 50 bases, less than 40 bases, or less than 30 bases.

In some instances, the determined risk scores are used to select, initiate, adjust, or terminate a treatment for cancer in the subject from which the sample was derived, as described elsewhere herein.

In some instances, the disclosed systems may further comprise sample processing and library preparation workstations, microplate-handling robotics, fluid dispensing systems, temperature control modules, environmental control chambers, additional data storage modules, data communication modules (e.g., Bluetooth®, WiFi, intranet, or internet communication hardware and associated software), display modules, one or more local and/or cloud-based software packages (e.g., instrument/system control software packages, sequencing data analysis software packages), etc., or any combination thereof. In some instances, the systems may comprise, or be part of, a computer system or computer network as described elsewhere herein.

### V. Computer Systems and Networks

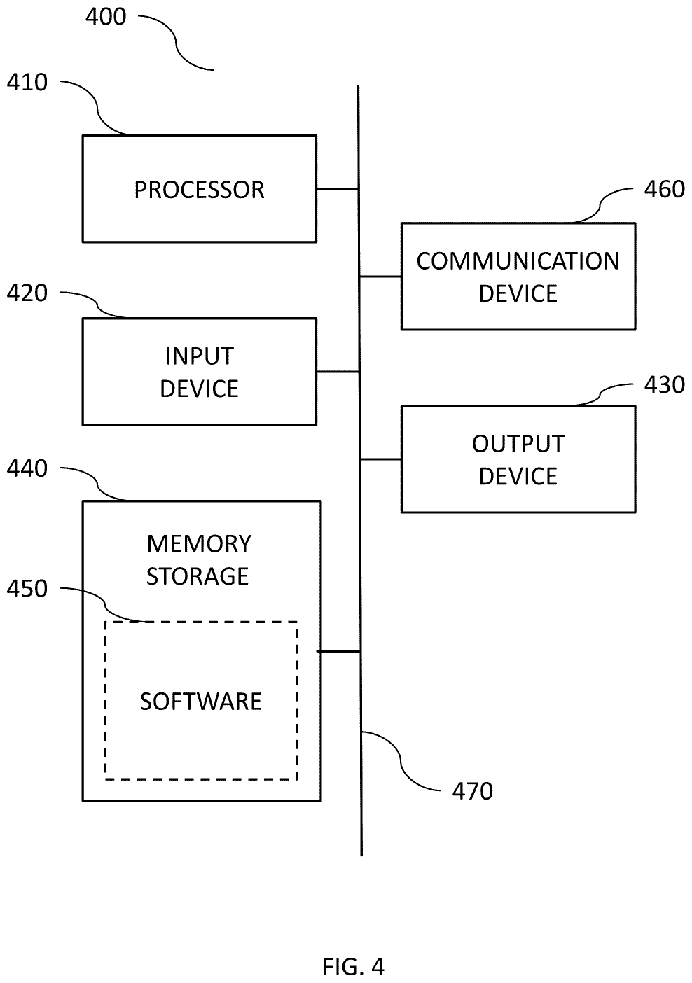

FIG. 4 illustrates an example of a computing device or system in accordance with one embodiment. Device 400 can be a host computer connected to a network. Device 400 can be a client computer or a server. As shown in FIG. 4, device 400 can be any suitable type of microprocessor-based device, such as a personal computer, workstation, server or handheld computing device (portable electronic device) such as a phone or tablet. The device can include, for example, one or more processor(s) 410, input devices 420, output devices 430, memory or storage devices 440, communication devices 460, and nucleic acid sequencers 470. Software 450 residing in memory or storage device 440 may comprise, e.g., an operating system as well as software for executing the methods described herein. Input device 420 and output device 430 can generally correspond to those described herein, and can either be connectable or integrated with the computer.

Input device 420 can be any suitable device that provides input, such as a touch screen, keyboard or keypad, mouse, or voice-recognition device. Output device 430 can be any suitable device that provides output, such as a touch screen, haptics device, or speaker.

Storage 440 can be any suitable device that provides storage (e.g., an electrical, magnetic or optical memory including a RAM (volatile and non-volatile), cache, hard drive, or removable storage disk). Communication device 460 can include any suitable device capable of transmitting and receiving signals over a network, such as a network interface chip or device. The components of the computer can be connected in any suitable manner, such as via a wired media (e.g., a physical system bus 480, Ethernet connection, or any other wire transfer technology) or wirelessly (e.g., Bluetooth®, Wi-Fi®, or any other wireless technology).

Software module 450, which can be stored as executable instructions in storage 440 and executed by processor(s) 410, can include, for example, an operating system and/or the processes that embody the functionality of the methods of the present disclosure (e.g., as embodied in the devices as described herein).

Software module 450 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 440, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Software module 450 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

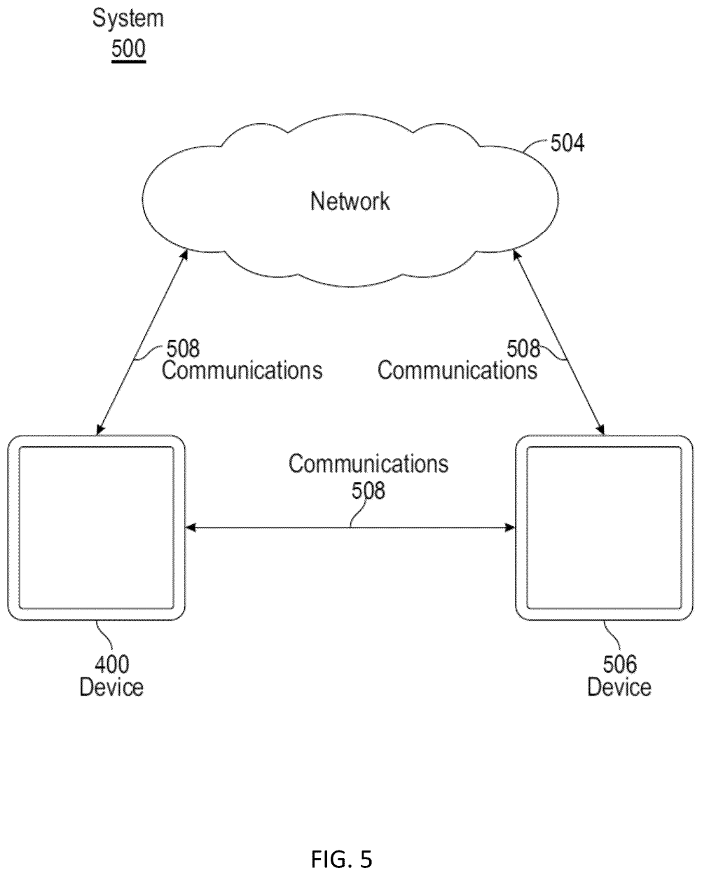

Device 400 may be connected to a network (e.g., network 504, as shown in FIG. 5 and/or described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, Tl or T3 lines, cable networks, DSL, or telephone lines.

Device 400 can be implemented using any operating system, e.g., an operating system suitable for operating on the network. Software module 450 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example. In some embodiments, the operating system is executed by one or more processors, e.g., processor(s) 410.

Device 400 can further include a sequencer 470, which can be any suitable nucleic acid sequencing instrument.

FIG. 5 illustrates an example of a computing system in accordance with one embodiment. In system 500, device 400 (e.g., as described above and illustrated in FIG. 4) is connected to network 504, which is also connected to device 506. In some embodiments, device 506 is a sequencer. Exemplary sequencers can include, without limitation, Roche/454's Genome Sequencer (GS) FLX System, Illumina/Solexa's Genome Analyzer (GA), Illumina's HiSeq® 2500, HiScq® 3000, HiScq® 4000 and NovaSeq® 6000 Sequencing Systems, Life/APG's Support Oligonucleotide Ligation Detection (SOLID) system, Polonator's G.007 system, Helicos BioSciences' HeliScope Gene Sequencing system, or Pacific Biosciences' PacBio® RS system.

Devices 400 and 506 may communicate, e.g., using suitable communication interfaces via network 504, such as a Local Area Network (LAN), Virtual Private Network (VPN), or the Internet. In some embodiments, network 504 can be, for example, the Internet, an intranet, a virtual private network, a cloud network, a wired network, or a wireless network. Devices 400 and 506 may communicate, in part or in whole, via wireless or hardwired communications, such as Ethernet, IEEE 802.11b wireless, or the like. Additionally, devices 400 and 506 may communicate, e.g., using suitable communication interfaces, via a second network, such as a mobile/cellular network. Communication between devices 400 and 506 may further include or communicate with various servers such as a mail server, mobile server, media server, telephone server, and the like. In some embodiments, Devices 400 and 506 can communicate directly (instead of, or in addition to, communicating via network 504), e.g., via wireless or hardwired communications, such as Ethernet, IEEE 802.11b wireless, or the like. In some embodiments, devices 400 and 506 communicate via communications 508, which can be a direct connection or can occur via a network (e.g., network 504).

One or all of devices 400 and 506 generally include logic (e.g., http web server logic) or are programmed to format data, accessed from local or remote databases or other sources of data and content, for providing and/or receiving information via network 504 according to various examples described herein.

### VI. Exemplary Implementations

The following implementations of the disclosed methods are exemplary and are not intended to limit the scope of the invention.
Clause 1: A method of treating a subject having a cancer with gemcitabine plus albumin-bound paclitaxel comprising (a) determining a risk score for the subject based on aneuploidy status or loss of heterozygosity (LOH) for one or more subgenomic intervals in a sample from the subject, and (b) treating the subject with gemcitabine plus albumin-bound paclitaxel if the risk score is less than a predetermined threshold.
Clause 2: A method of selecting a treatment for a subject having a cancer, the method comprising determining a risk score for the subject based on aneuploidy status or loss of heterozygosity (LOH) for one or more subgenomic intervals in a sample from the subject, wherein if the risk score is less than a predetermined threshold, the subject is identified as one who may benefit from treatment with gemcitabine plus albumin-bound paclitaxel.
Clause 3: A method of identifying a subject having a cancer for treatment with gemcitabine plus albumin-bound paclitaxel comprising (a) determining a risk score for the subject based on aneuploidy status or loss of heterozygosity (LOH) for one or more subgenomic intervals in a sample from the subject, and (b) identifying the subject for treatment with gemcitabine plus albumin-bound paclitaxel if the risk score is less than a predetermined threshold.
Clause 4: A method of identifying one or more treatment options for a subject having a cancer, the method comprising (a) determining a risk score for the subject based on aneuploidy status or loss of heterozygosity (LOH) for one or more subgenomic intervals in a sample from the subject, and (b) generating a report comprising one or more treatment options identified for the subject based at least in part on the risk score determined for the sample, wherein if the risk score is less than a predetermined threshold, the subject is identified as one who may benefit from treatment with gemcitabine plus albumin-bound paclitaxel.
Clause 5: A method of predicting survival of a subject having cancer, the method comprising determining a risk score for the subject based on aneuploidy status or loss of heterozygosity (LOH) for one or more subgenomic intervals in a sample from the subject, wherein if the risk score is less than a predetermined threshold, the subject is predicted to have longer survival when treated with gemcitabine plus albumin-bound paclitaxel, as compared to a subject that is not treated with gemtabicine plus albumin-bound paclitaxel.
Clause 6: A method of monitoring, evaluating, or screening a subject having a cancer, the method comprising determining a risk score for the subject based on aneuploidy status or loss of heterozygosity (LOH) for one or more subgenomic intervals in a sample from the subject, wherein if the risk score is less than a predetermined threshold, the subject is predicted to have longer survival when treated with gemcitabine plus albumin-bound paclitaxel, as compared to a subject that was not treated with gemcitabine plus albumin-bound paclitaxel.
Clause 7: A method of predicting survival of a subject having cancer, the method comprising determining a risk score for the subject based on aneuploidy status or loss of heterozygosity (LOH) for one or more subgenomic intervals in a sample from the subject, wherein if the risk score is less than a predetermined threshold, the subject is predicted to have longer survival when treated with gemcitabine plus albumin-bound paclitaxel, as compared to a subject with a risk score that is greater than the predetermined threshold.
Clause 8: A method of monitoring, evaluating, or screening a subject having a cancer, the method comprising determining a risk score for the subject based on aneuploidy status or loss of heterozygosity (LOH) for one or more subgenomic intervals in a sample from the subject, wherein if the risk score is less than a predetermined threshold, the subject is predicted to have longer survival when treated with gemcitabine plus albumin-bound paclitaxel, as compared to a subject with a risk score that is greater than the predetermined threshold.
Clause 9: A method of stratifying a subject with cancer for treatment with gemcitabine plus albumin-bound paclitaxel, the method comprising determining a risk score for the subject based on aneuploidy status or loss of heterozygosity (LOH) for one or more subgenomic intervals in a sample from the subject, wherein if the risk score is less than a predetermined threshold, the subject is treated with gemcitabine plus albumin-bound paclitaxel, and wherein if the risk score is greater than the predetermined threshold, the subject is treated with a different anti-cancer therapy.
Clause 10: A method of treating a subject having a cancer with FOLFIRINOX comprising (a) determining a risk score for the subject based on aneuploidy status or loss of heterozygosity (LOH) for one or more subgenomic intervals in a sample from the subject, and (b) treating the subject with FOLFIRINOX if the risk score is less than a predetermined threshold.
Clause 11: A method of selecting a treatment for a subject having a cancer, the method comprising determining a risk score for the subject based on aneuploidy status or loss of heterozygosity (LOH) for one or more subgenomic intervals in a sample from the subject, wherein if the risk score is less than a predetermined threshold, the subject is identified as one who may benefit from treatment with FOLFIRINOX.
Clause 12: A method of identifying a subject having a cancer for treatment with FOLFIRINOX comprising (a) determining a risk score for the subject based on aneuploidy status or loss of heterozygosity (LOH) for one or more subgenomic intervals in a sample from the subject, and (b) identifying the subject for treatment with FOLFIRINOX if the risk score is less than a predetermined threshold.
Clause 13: A method of identifying one or more treatment options for a subject having a cancer, the method comprising (a) determining a risk score for the subject based on aneuploidy status or loss of heterozygosity (LOH) for one or more subgenomic intervals in a sample from the subject, and (b) generating a report comprising one or more treatment options identified for the subject based at least in part on the risk score determined for the sample, wherein if the risk score is less than a predetermined threshold, the subject is identified as one who may benefit from treatment with FOLFIRINOX.
Clause 14: A method of predicting survival of a subject having cancer, the method comprising determining a risk score for the subject based on aneuploidy status or loss of heterozygosity (LOH) for one or more subgenomic intervals in a sample from the subject, wherein if the risk score is less than a predetermined threshold, the subject is predicted to have longer survival when treated with FOLFIRINOX, as compared to a subject that is not treated with FOLFIRINOX.
Clause 15: A method of monitoring, evaluating, or screening a subject having a cancer, the method comprising determining a risk score for the subject based on aneuploidy status or loss of heterozygosity (LOH) for one or more subgenomic intervals in a sample from the subject, wherein if the risk score is less than a predetermined threshold, the subject is predicted to have longer survival when treated with FOLFIRINOX, as compared to a subject that was not treated with FOLFIRINOX.
Clause 16: A method of predicting survival of a subject having cancer, the method comprising determining a risk score for the subject based on aneuploidy status or loss of heterozygosity (LOH) for one or more subgenomic intervals in a sample from the subject, wherein if the risk score is less than a predetermined threshold, the subject is predicted to have longer survival when treated with FOLFIRINOX, as compared to a subject with a risk score that is greater than the predetermined threshold.
Clause 17: A method of monitoring, evaluating, or screening a subject having a cancer, the method comprising determining a risk score for the subject based on aneuploidy status or loss of heterozygosity (LOH) for one or more subgenomic intervals in a sample from the subject, wherein if the risk score is less than a predetermined threshold, the subject is predicted to have longer survival when treated with FOLFIRINOX, as compared to a subject with a risk score that is greater than the predetermined threshold.
Clause 18: A method of stratifying a subject with cancer for treatment with FOLFIRINOX, the method comprising determining a risk score for the subject based on aneuploidy status or loss of heterozygosity (LOH) for one or more subgenomic intervals in a sample from the subject, wherein if the risk score is less than a predetermined threshold, the subject is treated with FOLFIRINOX, and wherein if the risk score is greater than the predetermined threshold, the subject is treated with a different anti-cancer therapy.
Clause 19: A method of treating a subject having a cancer with a first anti-cancer therapy comprising (a) determining a risk score for the subject based on aneuploidy status or loss of heterozygosity (LOH) for one or more subgenomic intervals in a sample from the subject, and (b) treating the subject with the first anti-cancer therapy if the risk score is less than a predetermined threshold.
Clause 20: A method of selecting a first anti-cancer therapy for a subject having a cancer, the method comprising determining a risk score for the subject based on aneuploidy status or loss of heterozygosity (LOH) for one or more subgenomic intervals in a sample from the subject, wherein if the risk score is less than a predetermined threshold, the subject is identified as one who may benefit from treatment with the first anti-cancer therapy.
Clause 21: A method of identifying a subject having a cancer for treatment with a first anti-cancer therapy comprising (a) determining a risk score for the subject based on aneuploidy status or loss of heterozygosity (LOH) for one or more subgenomic intervals in a sample from the subject, and (b) identifying the subject for treatment with the first anti-cancer therapy if the risk score is less than a predetermined threshold.
Clause 22: A method of identifying one or more treatment options for a subject having a cancer, the method comprising (a) determining a risk score for the subject based on aneuploidy status or loss of heterozygosity (LOH) for one or more subgenomic intervals in a sample from the subject, and (b) generating a report comprising one or more treatment options identified for the subject based at least in part on the risk score determined for the sample, wherein if the risk score is less than a predetermined threshold, the subject is identified as one who may benefit from treatment with a first anti-cancer therapy.
Clause 23: A method of predicting survival of a subject having cancer, the method comprising determining a risk score for the subject based on aneuploidy status or loss of heterozygosity (LOH) for one or more subgenomic intervals in a sample from the subject, wherein if the risk score is less than a predetermined threshold, the subject is predicted to have longer survival when treated with a first anti-cancer therapy, as compared to a subject that is not treated with the first anti-cancer therapy.
Clause 24: A method of monitoring, evaluating, or screening a subject having a cancer, the method comprising determining a risk score for the subject based on ancuploidy status or loss of heterozygosity (LOH) for one or more subgenomic intervals in a sample from the subject, wherein if the risk score is less than a predetermined threshold, the subject is predicted to have longer survival when treated with a first anti-cancer therapy, as compared to a subject that was not treated with the first anti-cancer therapy.
Clause 25: A method of predicting survival of a subject having cancer, the method comprising determining a risk score for the subject based on aneuploidy status or loss of heterozygosity (LOH) for one or more subgenomic intervals in a sample from the subject, wherein if the risk score is less than a predetermined threshold, the subject is predicted to have longer survival when treated with a first anti-cancer therapy, as compared to a subject with a risk score that is greater than the predetermined threshold.
Clause 26: A method of monitoring, evaluating, or screening an subject having a cancer, the method comprising determining a risk score for the subject based on aneuploidy status or loss of heterozygosity (LOH) for one or more subgenomic intervals in a sample from the subject, wherein if the risk score is less than a predetermined threshold, the subject is predicted to have longer survival when treated with a first anti-cancer therapy, as compared to a subject with a risk score that is greater than the predetermined threshold.
Clause 27: A method of stratifying a subject with cancer for treatment with a first anti-cancer therapy, the method comprising determining a risk score for the subject based on ancuploidy status or loss of heterozygosity (LOH) for one or more subgenomic intervals in a sample from the subject, wherein if the risk score is less than a predetermined threshold, the subject is treated with the first anti-cancer therapy, and wherein if the risk score is greater than the predetermined threshold, the subject is treated with a second anti-cancer therapy.
Clause 28: The method of any one of clauses 19-27, wherein the first anti-cancer therapy is a chemotherapy or an immune-oncology (IO) therapy.
Clause 29: The method of clause 28, wherein the chemotherapy comprises one or more of an alkylating agent, an alkyl sulfonates aziridine, an ethylenimine, a methylamelamine, an acetogenin, a camptothecin, a bryostatin, a callystatin, CC-1065, a cryptophycin, aa dolastatin, a duocarmycin, a cleutherobin, a pancratistatin, a sarcodictyin, a spongistatin, a nitrogen mustard, a nitrosurcas, an antibiotic, a dynemicin, a bisphosphonate, an esperamicina a neocarzinostatin chromophore or a related chromoprotein enediyne antiobiotic chromophore, an anti-metabolite, a folic acid analogue, a purine analog, a pyrimidine analog, an androgens, an anti-adrenal, a folic acid replenisher, aldophosphamide glycoside, aminolevulinic acid, eniluracil, amsacrine, bestrabucil, bisantrene, edatraxate, defofamine, demecolcine, diaziquone, elformithine, elliptinium acetate, an epothilone, ctoglucid, gallium nitrate, hydroxyurea, lentinan, lonidainine, maytansinoids, mitoguazone, mitoxantrone, mopidanmol, nitracrine, pentostatin, phenamet, pirarubicin, losoxantrone, podophyllinic acid, 2-ethylhydrazide, procarbazine, a PSK polysaccharide complex, razoxane, rhizoxin, sizofiran, spirogermanium, tenuazonic acid, triaziquone, 2,2′,2″-trichlorotriethylamine, a trichothecene, urethan, vindesine, dacarbazine, mannomustine, mitobronitol, mitolactol, pipobroman, gacytosine, arabinoside (“Ara-C”), cyclophosphamide, a taxoid, 6-thioguanine, mercaptopurine, a platinum coordination complex, vinblastine, platinum, etoposide (VP-16), ifosfamide, mitoxantrone, vincristine, vinorelbine, novantrone, teniposide, edatrexate, daunomycin, aminopterin, xeloda, ibandronate, irinotecan, topoisomerase inhibitor RFS 2000, difluorometlhylomithine (DMFO), a retinoid, capecitabine, carboplatin, procarbazine, plicomycin, gemcitabine, navelbine, farnesyl-protein transferase inhibitors, transplatinum, or any combination thereof.
Clause 30: The method of clause 28, wherein the IO therapy comprises a small molecule inhibitor, an antibody, a nucleic acid, an antibody-drug conjugate, a recombinant protein, a fusion protein, a natural compound, a peptide, a PROteolysis-TArgeting Chimera (PROTAC), a cellular therapy, a treatment for cancer being tested in a clinical trial, an immunotherapy, or any combination thereof.
Clause 31: The method of any one of clauses 19-27, wherein the first anti-cancer therapy comprises FOLFIRINOX, gemcitabine plus albumin-bound paclitaxel, gemcitabine, capecitabine, fluorouacil plus irinotecan liposomal and leucovorin, FOLFIRI, or capecitabine plus gemcitabine.
Clause 32: The method of any one of clauses 1-31, wherein risk score is calculated by a method comprising: obtaining genomic data comprising aneuploidy status or loss of heterozygosity data for one or more subgenomic intervals in a sample from the subject; and analyzing the genomic data for the subject using a model configured to receive genomic data comprising aneuploidy status or loss of heterozygosity (LOH) data the one or more subgenomic intervals identified in the subject and output a risk score for the subject, wherein the aneuploidy or LOH data are associated with a patient survival metric, and wherein the risk score predicts a response to a selected treatment for the subject.
Clause 33: The method of clause 32, further comprising converting the risk score output by the model for the subject to a binary (high-low) risk score based on a comparison to the predetermined threshold.
Clause 34: The method of clause 33, wherein the predetermined threshold is defined as a mean, median, or mode of risk scores calculated for a patient cohort used to train the model.
Clause 35: The method of clause 33, wherein the predetermined threshold is defined by a risk score value that maximizes a log-rank statistic for risk scores calculated for a patient cohort used to train the model.
Clause 36: The method of clause 33, wherein a low risk score indicates that the subject is likely to survive longer than a subject with a high risk score if treated with the selected treatment.
Clause 37: The method of any one of clauses 32-36, wherein the genomic data is based on sequence read data derived from a comprehensive genomic profiling assay.
Clause 38: The method of any one of clauses 32-37, wherein analyzing the genomic data for the subject further comprises analysis of clinical feature data for the subject.
Clause 39: The method of clause 38, wherein the clinical feature data comprises patient age, patient sex, patient race, patient clinical history, or any combination thereof.
Clause 40: The method of any one of clauses 32-39, wherein analyzing the genomic data for the subject further comprises analysis of Eastern Cooperative Oncology Group (ECOG) performance data for the subject.
Clause 41: The method of any one of clauses 32-40, wherein the model is a machine learning model.
Clause 42: The method of clause 41, wherein the machine learning model comprises a multivariable Cox proportional hazards regression model.
Clause 43: The method of clause 41 or clause 42, wherein the machine learning model comprises a conditional inference forest model.
Clause 44: The method of any one of clauses 32-43, wherein the risk score is for treatment with FOLFIRINOX, and the one or more subgenomic intervals for which aneuploidy or LOH correlated with a patient survival metric comprise Chr3q, Chr4p, Chr5p, Chr5q, Chr7q, Chr11p, Chr12p, Chr12q, Chr15q, Chr16p, Chr17p, Chr19p, Chr19q, Chr20p, Chr22q, or any combination thereof.
Clause 45: The method of any one of clauses 32-43, wherein the risk score is for treatment with FOLFIRINOX, and the one or more subgenomic intervals for which aneuploidy or LOH correlated with a patient survival metric comprise Chr7q, Chr15q, or any combination thereof.
Clause 46: The method of any one of clauses 32-43, wherein the risk score is for treatment with gemcitabine plus albumin-bound paclitaxel, and the one or more subgenomic intervals for which aneuploidy or LOH correlated with a patient survival metric comprise Chr1p, Chr1q, Chr3p, Chr6p, Chr6q, Chr7p, Chr7q, Chr8q, Chr9p, Chr9q, chr14q, Chr15q, Chr16p, chr17p, Chr17q, Chr18q, Chr19p, Chr20p, Chr21p, Chr21q, Chr22q, or any combination thereof.
Clause 47: The method of any one of clauses 32-43, wherein the risk score is for treatment with gemcitabine plus albumin-bound paclitaxel, and the one or more subgenomic intervals for which aneuploidy or LOH correlated with a patient survival metric comprise Chr3p, Chr6p, Chr8q, Chr9q, Chr18q, Chr20p, Chr21p, Chr21q, Chr22q, or any combination thereof.
Clause 48: The method of any one of clauses 32-44, wherein the patient survival metric comprises a hazard ratio, a progression free survival, an overall survival, a disease-free survival, an objective tumor response rate, a time to tumor progression, a time to treatment failure, a durable complete response, a time to next treatment, or any combination thereof.
Clause 49: The method of any one of clauses 2-9, 20-27 and 32-48, further comprising treating the subject with gemcitabine plus albumin-bound paclitaxel.
Clause 50: The method of any one of clauses 11-18, 20-27 and 32-48, further comprising treating the subject with FOLFIRINOX.
Clause 51: The method of any one of clauses 1, 10, and 32-50, further comprising treating the subject with an additional anti-cancer therapy.
Clause 52: The method of clause 51, wherein the additional anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, or any combination thereof.
Clause 53: The method of any one of clauses 1-52, wherein the sample comprises a tissue biopsy sample or a liquid biopsy sample.
Clause 54: The method of clause 53, wherein the sample is a tissue biopsy and comprises a tumor biopsy, tumor specimen, or circulating tumor cells.
Clause 55: The method of clause 53, wherein the sample is a liquid biopsy sample and comprises blood, serum, plasma, cerebrospinal fluid, sputum, stool, urine, or saliva.
Clause 56: The method of any one of clauses 1-55, wherein the sample comprises cells and/or nucleic acids from the cancer.
Clause 57: The method of clause 56, wherein the sample comprises mRNA, DNA, circulating tumor DNA (ctDNA), cell-free DNA, cell-free RNA from the cancer, or any combination thereof.
Clause 58: The method of clause 53, wherein the sample is a liquid biopsy sample and comprises circulating tumor cells (CTCs).
Clause 59: The method of clause 53, wherein the sample is a liquid biopsy sample and comprises cell-free DNA (cfDNA), circulating tumor DNA (ctDNA), or any combination thereof.
Clause 60: The method of any one of clauses 32-59, wherein the genomic data is based on sequence data derived from sequencing the sample from the subject.
Clause 61: The method of clause 60, wherein the sequencing comprises use of a massively parallel sequencing (MPS) technique, whole genome sequencing (WGS), whole exome sequencing, targeted sequencing, direct sequencing, next-generation sequencing (NGS), or a Sanger sequencing technique.
Clause 62: The method of clause 60 or clause 61, wherein the sequencing comprises: providing a plurality of nucleic acid molecules obtained from the sample, wherein the plurality of nucleic acid molecules comprises a mixture of tumor nucleic acid molecules and non-tumor nucleic acid molecules; optionally, ligating one or more adapters onto one or more nucleic acid molecules from the plurality of nucleic acid molecules; amplifying nucleic acid molecules from the plurality of nucleic acid molecules; optionally, capturing nucleic acid molecules from the amplified nucleic acid molecules, wherein the captured nucleic acid molecules are captured from the amplified nucleic acid molecules by hybridization to one or more bait molecules; and sequencing, by a sequencer, the captured nucleic acid molecules to obtain a plurality of sequence reads corresponding to one or more genomic loci within a subgenomic interval in the sample.
Clause 63: The method of clause 62, wherein the adapters comprise one or more of amplification primer sequences, flow cell adapter hybridization sequences, unique molecular identifier sequences, substrate adapter sequences, or sample index sequences.
Clause 64: The method of clause 62 or clause 63, wherein amplifying nucleic acid molecules comprises performing a polymerase chain reaction (PCR) technique, a non-PCR amplification technique, or an isothermal amplification technique.
Clause 65: The method of any one of clauses 62-64, wherein the one or more bait molecules comprise one or more nucleic acid molecules, each comprising a region that is complementary to a region of a captured nucleic acid molecule.
Clause 66: The method of clause 65, wherein the one or more bait molecules each comprise a capture moiety.
Clause 67: The method of clause 66, wherein the capture moiety is biotin.
Clause 68: The method of any one of clauses 1-67, wherein the cancer is a B cell cancer, a melanoma, breast cancer, lung cancer, bronchus cancer, colorectal cancer or carcinoma, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer or carcinoma, lung non-small cell lung carcinoma (NSCLC), head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.
Clause 69: The method of clause 68, wherein the cancer is pancreatic cancer.
Clause 70: The method of clauses 69, wherein the pancreatic cancer is metastatic pancreatic cancer.
Clause 71: The method of any of clauses 1-70, wherein the subject is a human.
Clause 72: The method of any one of clauses 1-71, wherein the subject has previously been treated with an anti-cancer therapy.
Clause 73: The method of clause 72, wherein the anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, or any combination thereof.

### Examples

### Example 1-Identification of Aneuploidy Biomarkers Associated with Response to First-Line Treatment of Metastatic Pancreatic Cancer

This section provides a non-limiting example of the identification of predictive biomarkers for response to first line treatment with the FOLFIRINOX regimen (a chemotherapy combination that includes the drugs leucovorin calcium (folinic acid), fluorouracil, irinotecan hydrochloride, and oxaliplatin) or gemcitabine plus paclitaxel in metastatic pancreatic cancer.

### Methods

### Patient Cohort

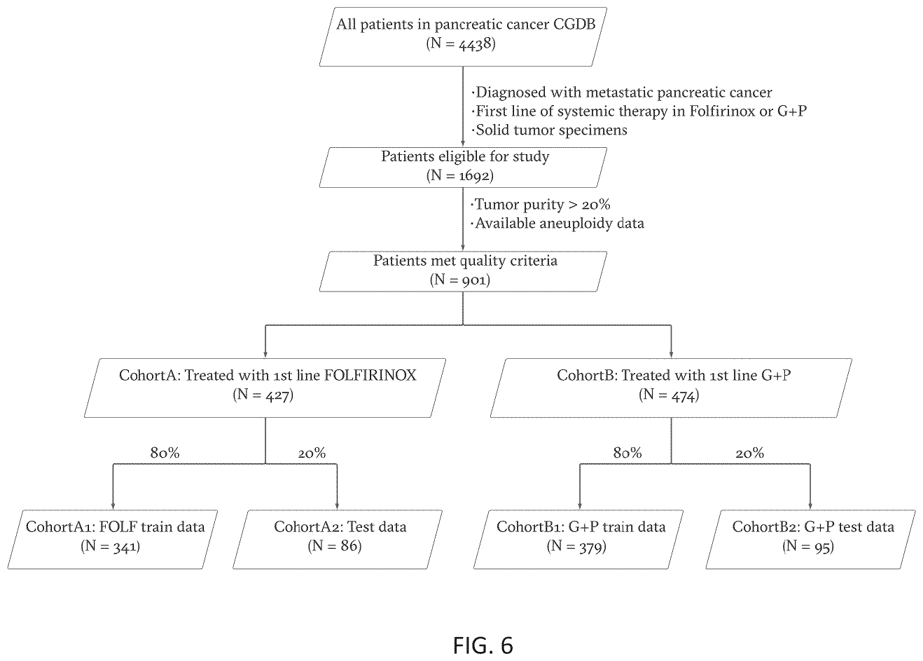

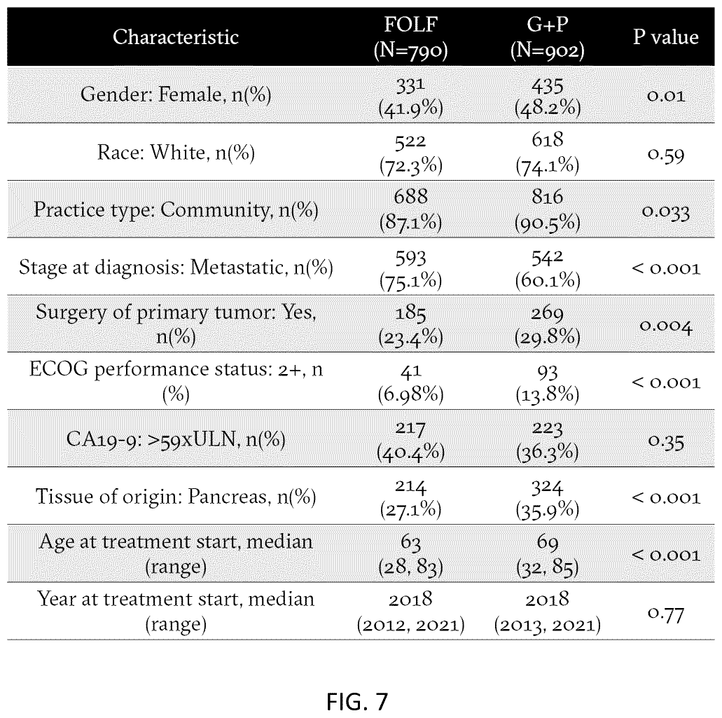

A cohort of 1,692 patients with metastatic pancreatic cancer who were treated with first-line FOLFIRINOX (FOLF, n=790) or gemcitabine plus albumin-bound paclitaxel (G+P, n=902) was chosen from a de-identified database (FIG. 6). The de-identified data originated from approximately 280 cancer clinics, representing around ˜800 sites of care, with the comprehensive genomic profiling performed on tumor samples from each patient as part of standard of care. The gain/loss status as well as loss of heterozygosity (LOH) status of each chromosome arm was assessed using a custom research-use only algorithm that utilizes copy number model calls for each segment and SNP mutant allele frequency (MAF) information from sequencing data. FIG. 7 shows a summary of the cohort demographics.

### Statistical Analysis and Modeling

Univariable Cox proportional hazards regression was used to identify chromosome arm-level aneuploidies associated with survival in patients treated with first-line FOLF or G+P.

For feature selection in each treatment cohort, a two repeat 10-fold covariate analysis was applied, and the bidirectional stepwise regression procedure was used to select aneuploidy features that associated with patient survival. A multivariate Cox model was built in each training dataset using aneuploidy features that appeared at a rate higher than 70% in the feature selection process. A binary risk stratification threshold was then calculated based on the linear predictor of the multivariable Cox model that maximized the log-rank test statistic in each training dataset, and categorized as high or low using a median threshold.

In some cases, a conditional inference survival forest (CIF) model was used instead of a multivariable Cox model. In each treatment cohort, a five-fold covariate analysis was applied to build a conditional inference survival forest (CIF) Integrated Brier score and Brier score were used to evaluate CIF model performance.

### Results

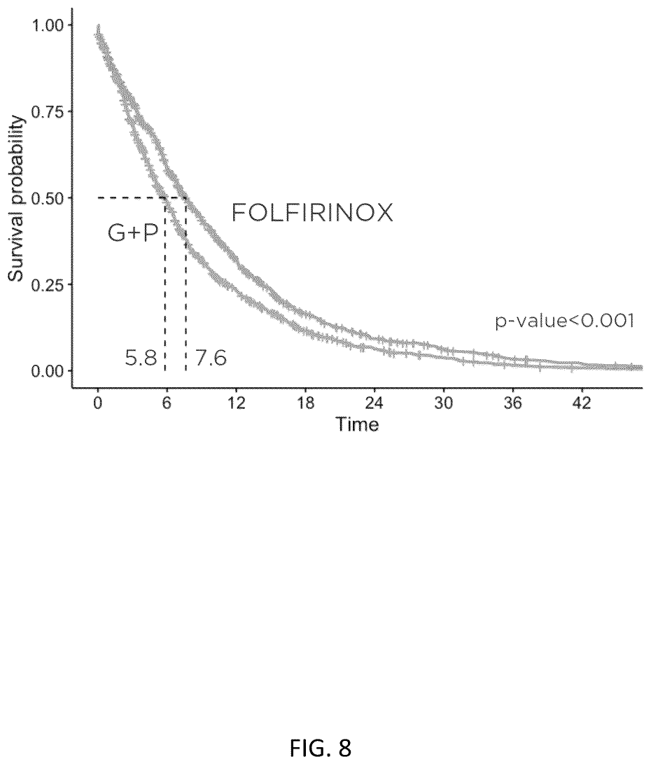

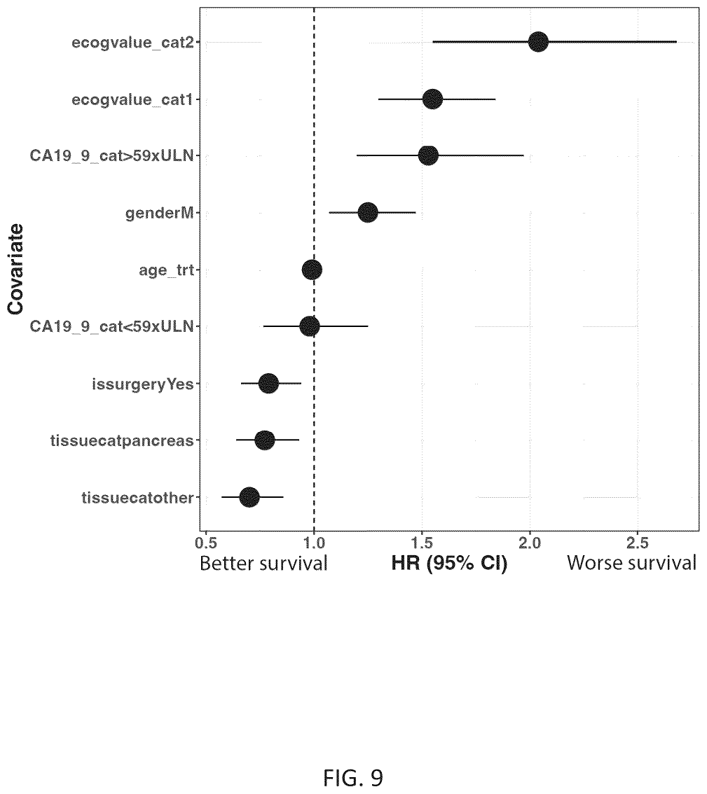

As shown in FIG. 8, there is a significant difference in patient survival observed for metastatic pancreatic cancer patients treated with either first-line FOLF or G+P. Patients treated with first-line FOLF treatment had better survival compared to patients with first-line G+P treatment in the real-world setting (hazard ratio [HR]=2.40, 95% confidence interval [CI]=1.46-3.93, p=<0.001). The median survival time is 7.6 (6.9-8.7) months in the first-line FOLF treatment cohort and 5.8 (5.1-6.5) months in the first-line G+P treatment cohort. FIG. 9 shows the association of patient demographics with survival, with some clinical characteristics associated with higher survival independent of treatment.

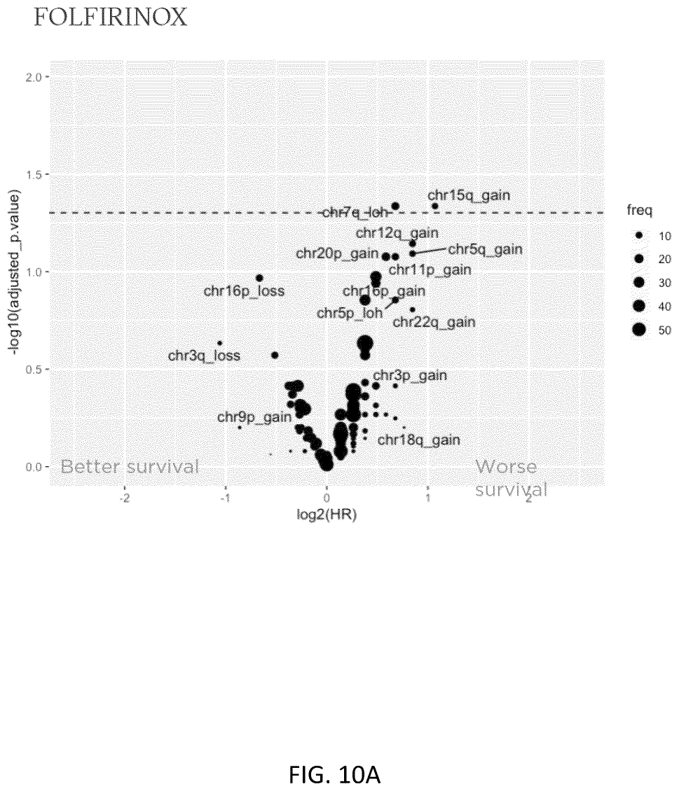

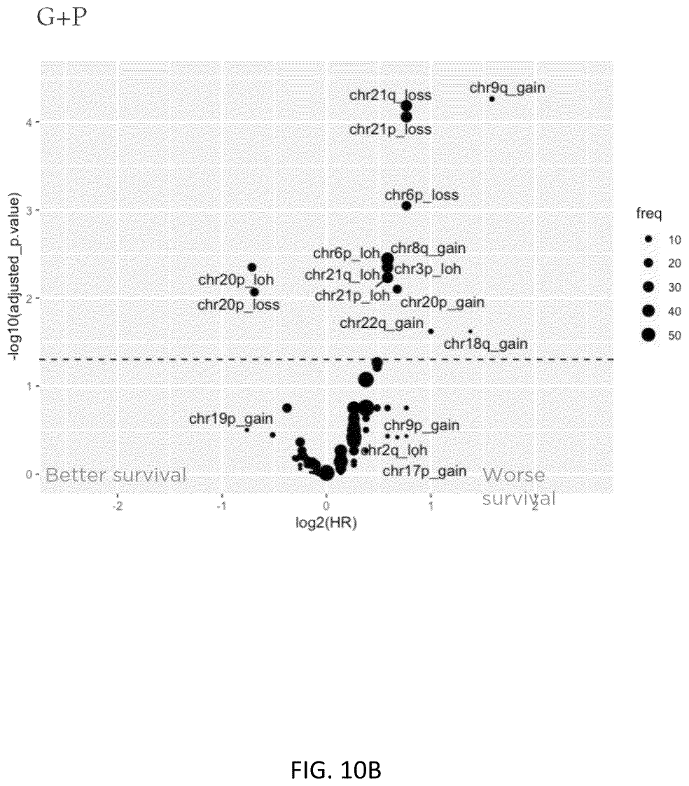

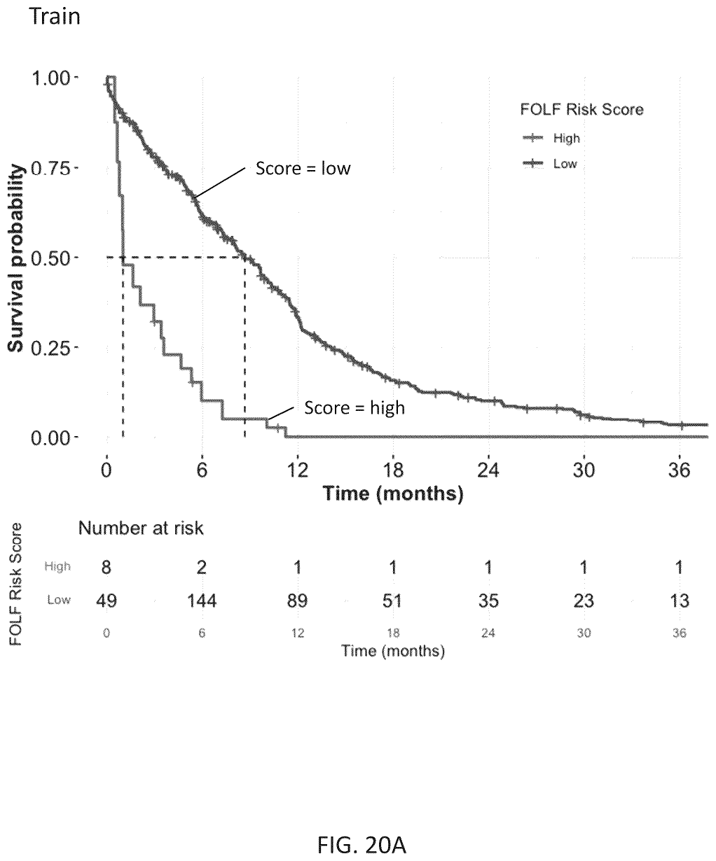

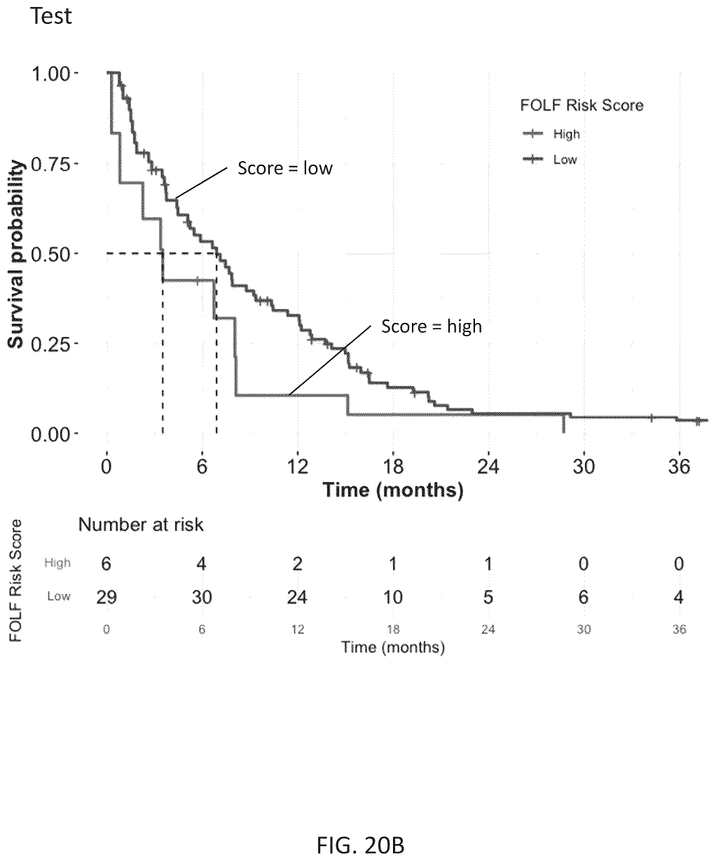

Among the FOLF-treated cohort, two aneuploidy features associated with survival were identified (Chr7q LOH, Chr15q gain; FDR adjusted p<0.05; FIG. 10A). A multivariate Cox regression model was used to calculate a FOLF risk score based on chromosome arm level aneuploidy features. FOLF-treated patients with a high FOLF risk score had worse survival compared to those with a low FOLF risk score in the training dataset (HR=5.11, CI=3.14-8.33, p=<0.001; FIG. 20A). This association was not significant among FOLF-treated patients in the test dataset (HR=1.54, CI=0.77-3.07, p=0.21; FIG. 20B).

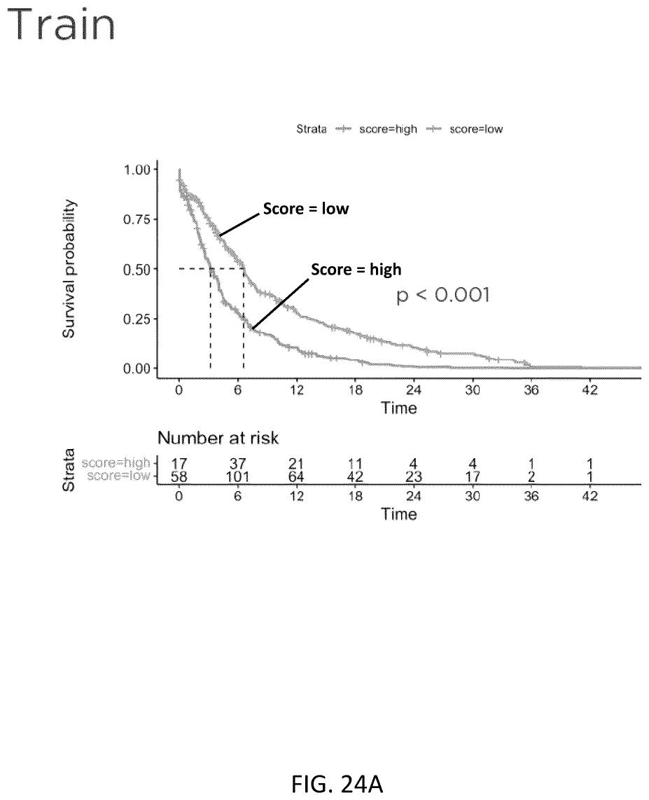

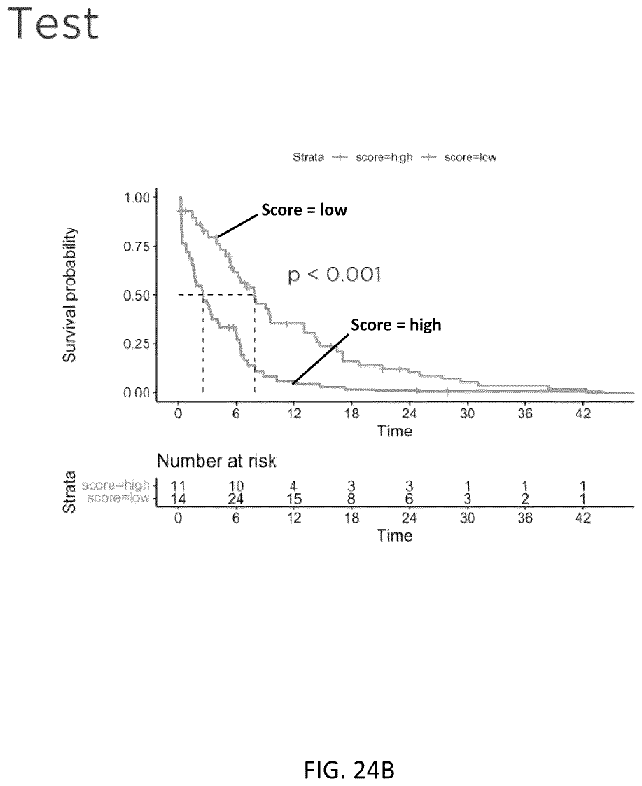

Among the G+P treated cohort, fourteen aneuploidy features were associated with survival (Chr3p LOH, Chr6p LOH, Chr6p loss, Chr8q gain, Chr9q gain, Chr18q gain, Chr20p LOH, Chr20p loss, Chr20p gain, Chr21p LOH,Chr21p loss, Chr21q loss, Chr21q LOH and Chr22q gain; FDR adjusted p<0.05; FIG. 10B). A multivariate Cox regression model was used to calculate a G+P risk score based on chromosome arm level aneuploidy features. G+P-treated patients with a high G+P risk score had worse survival compared to those with a low G+P risk score in the training dataset (HR=2.06, CI=1.62-2.61, p=<0.001; FIG. 24A). This association remained among G+P-treated patient in the test dataset (HR=2.40, CI=1.46-3.93, p=<0.001; FIG. 24B).

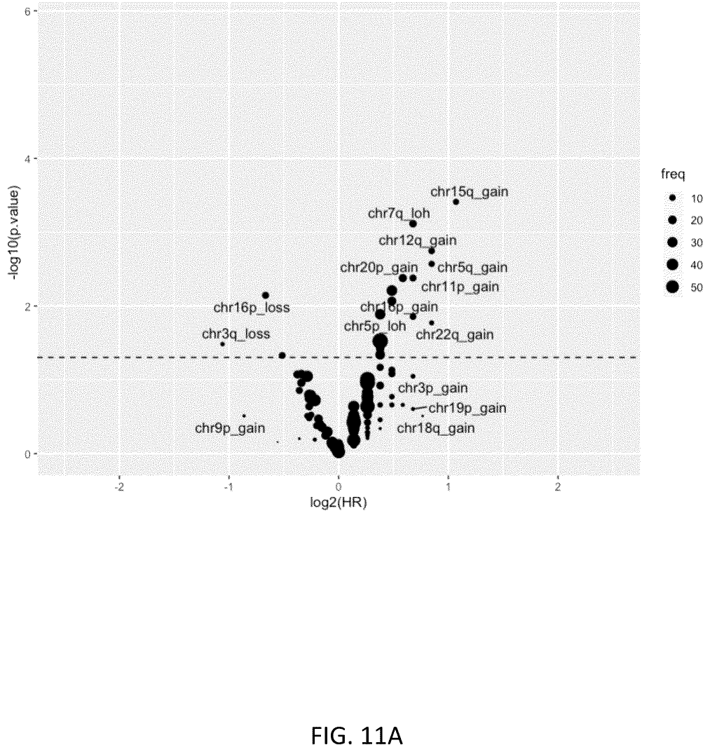

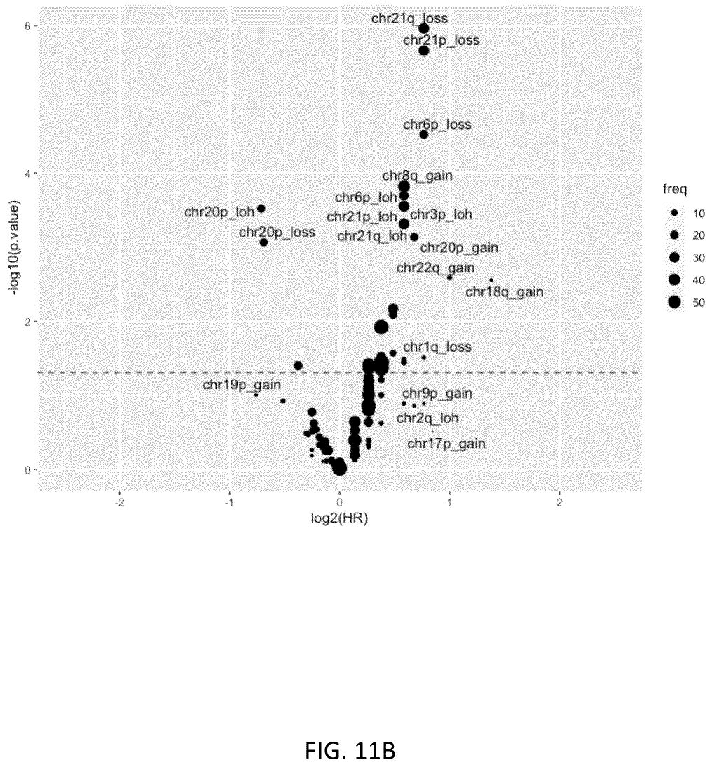

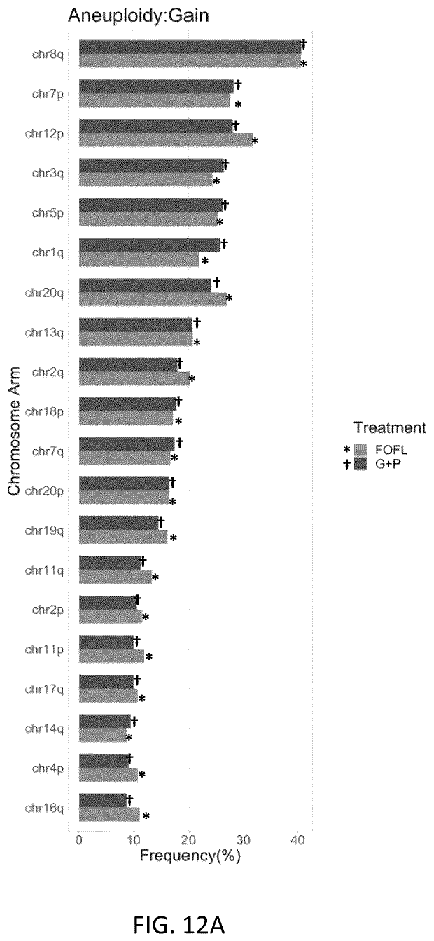

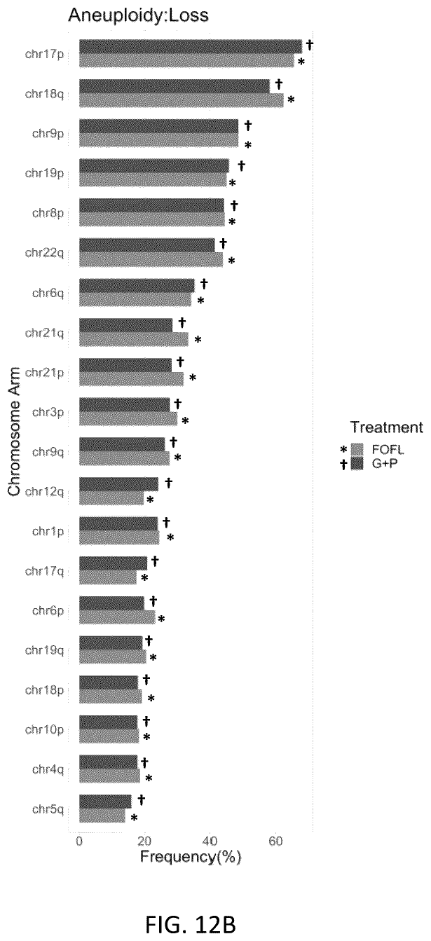

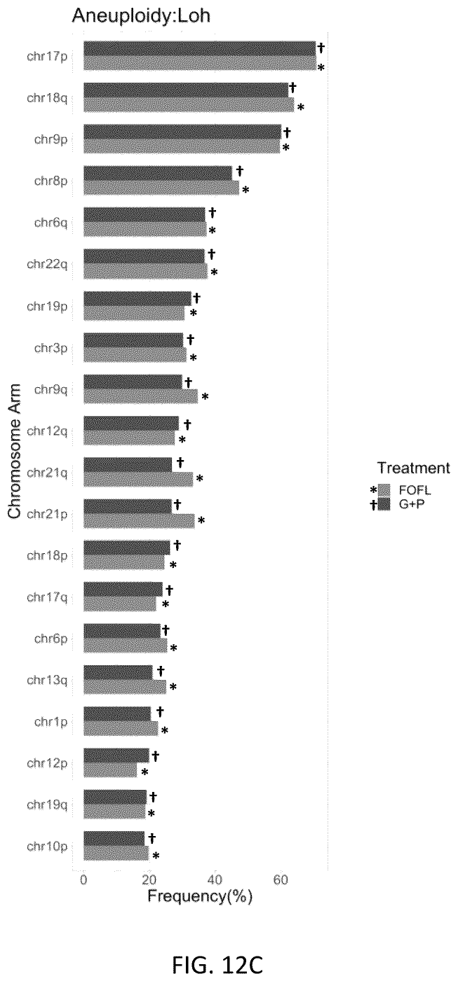

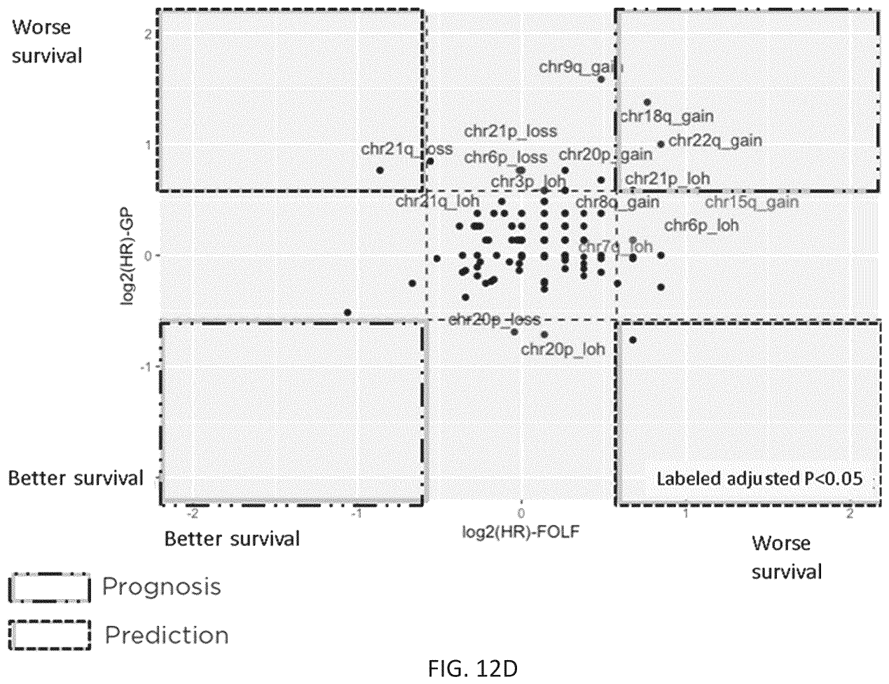

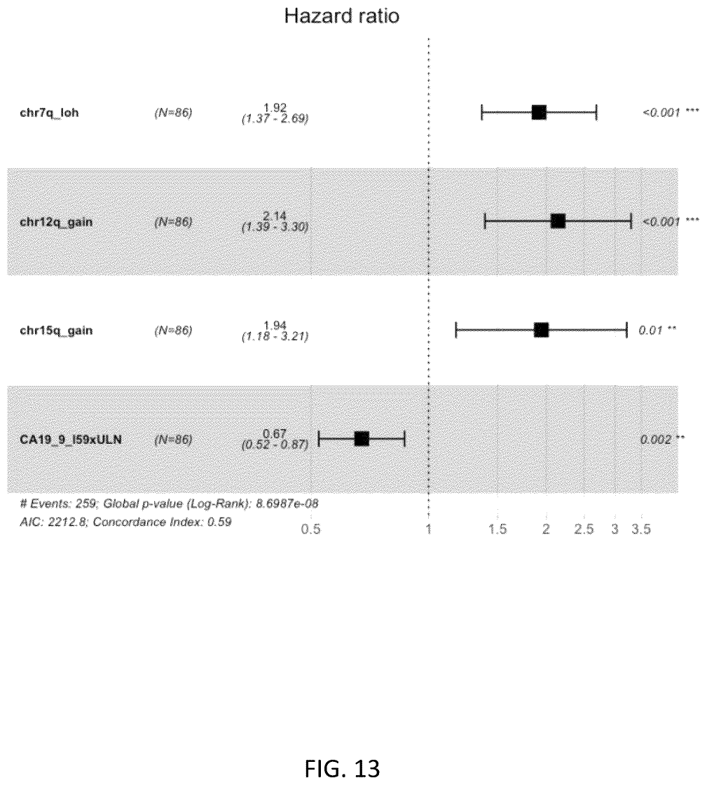

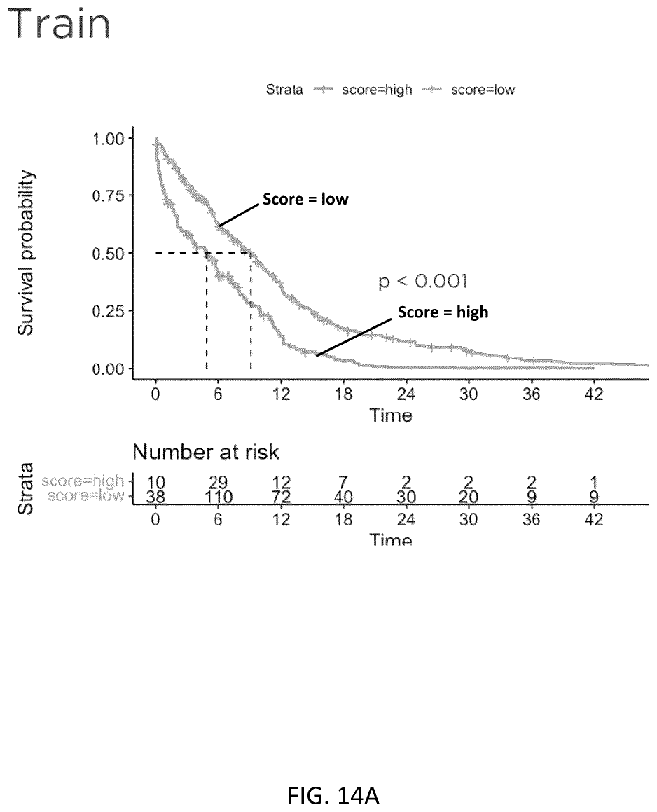

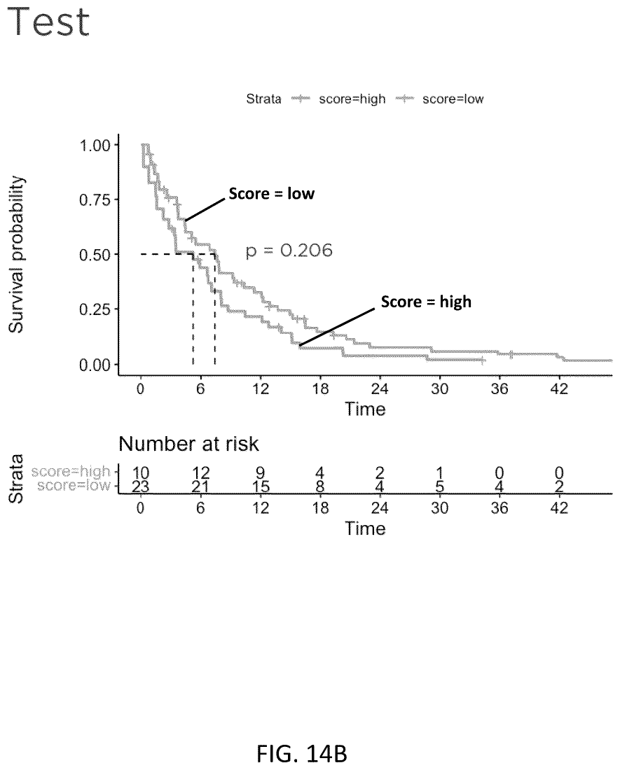

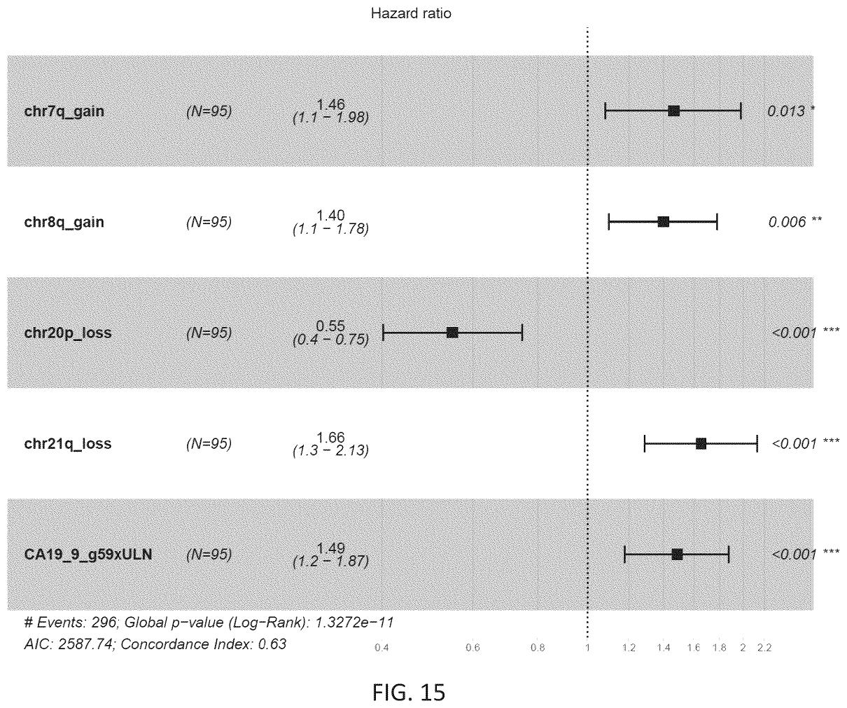

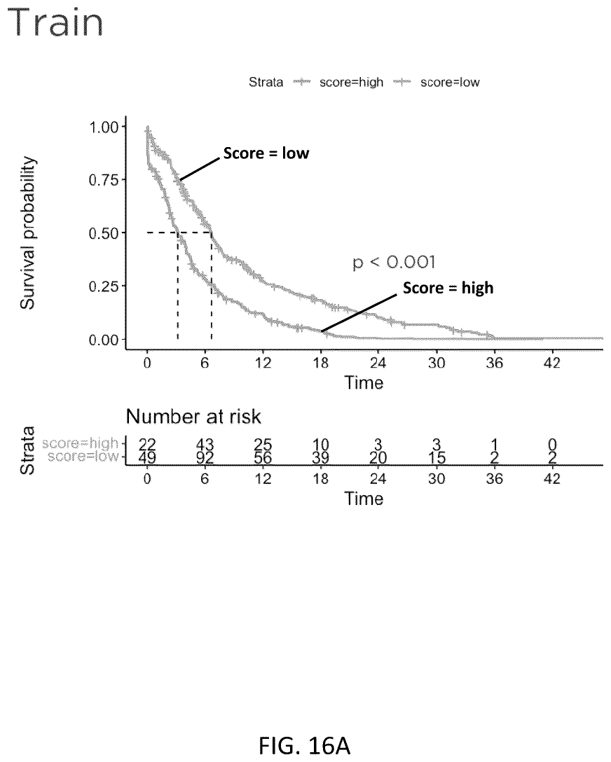

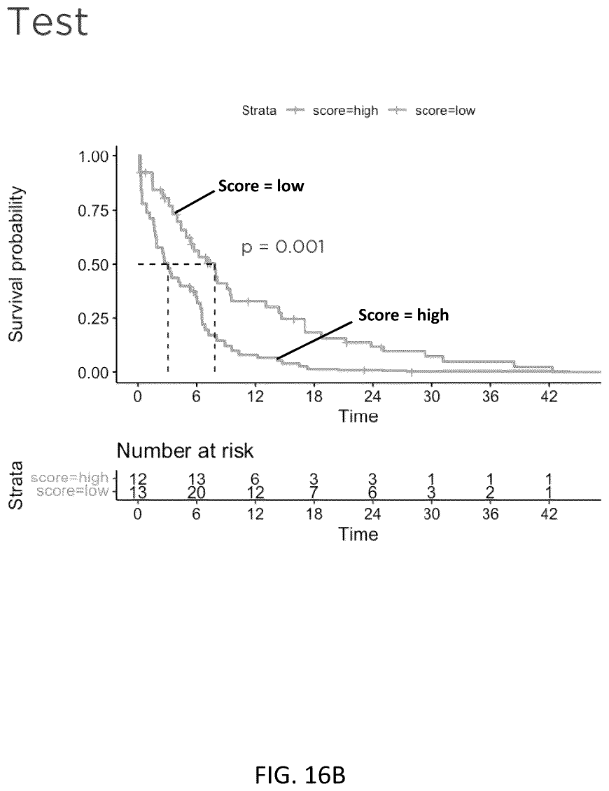

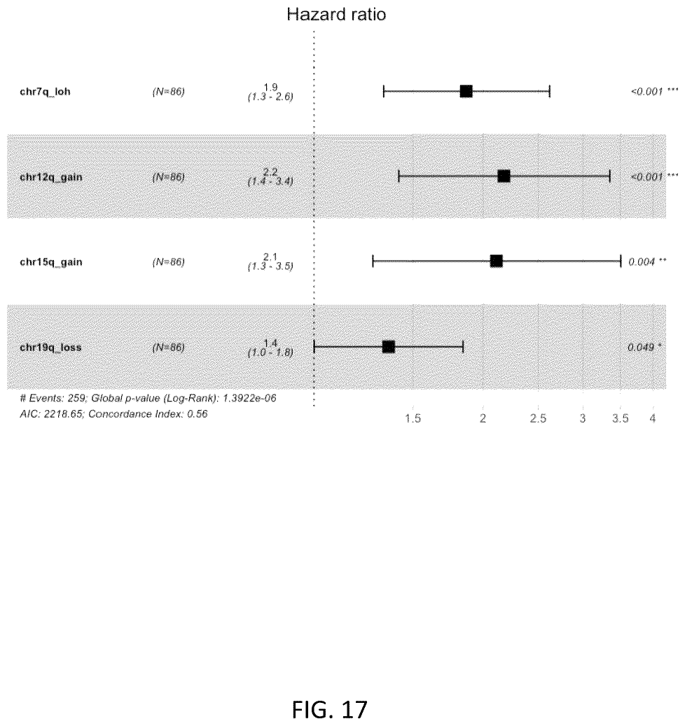

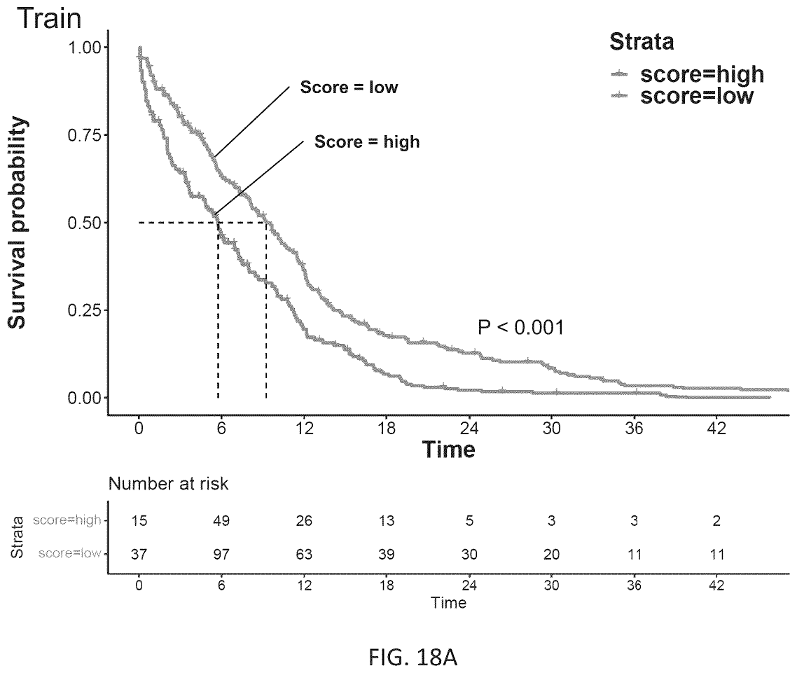

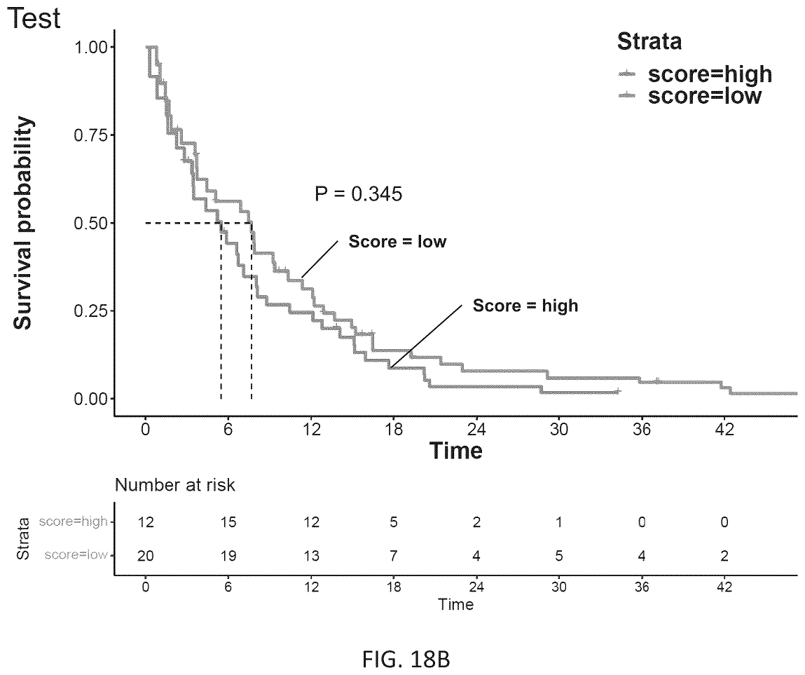

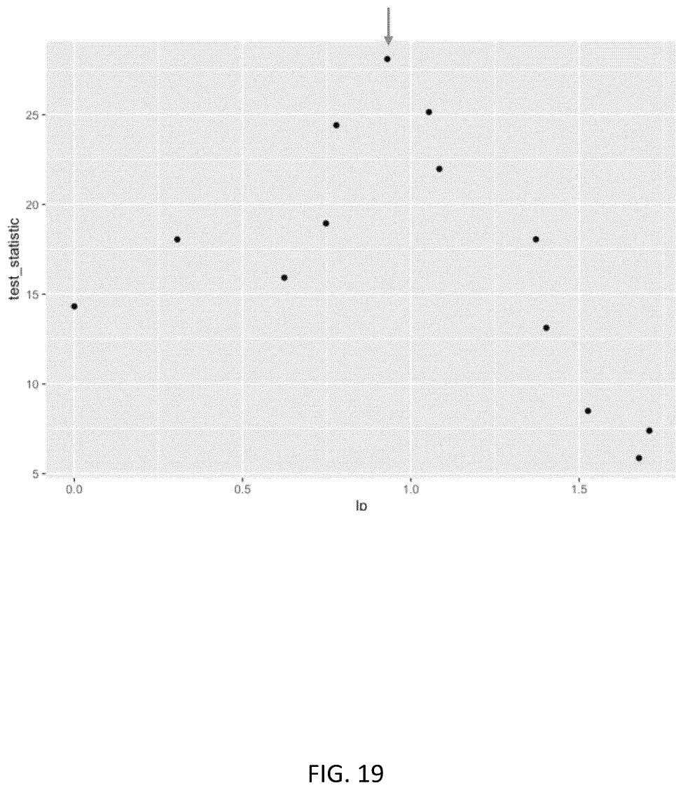

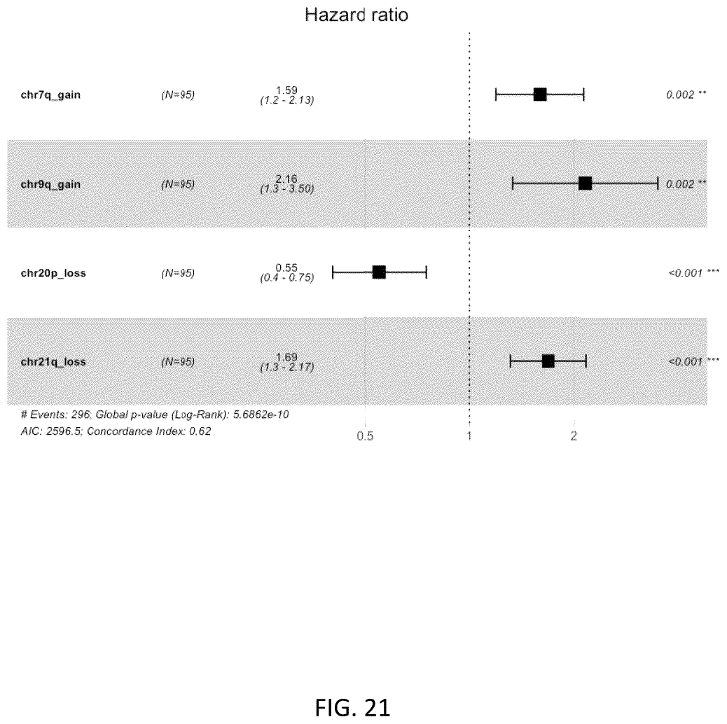

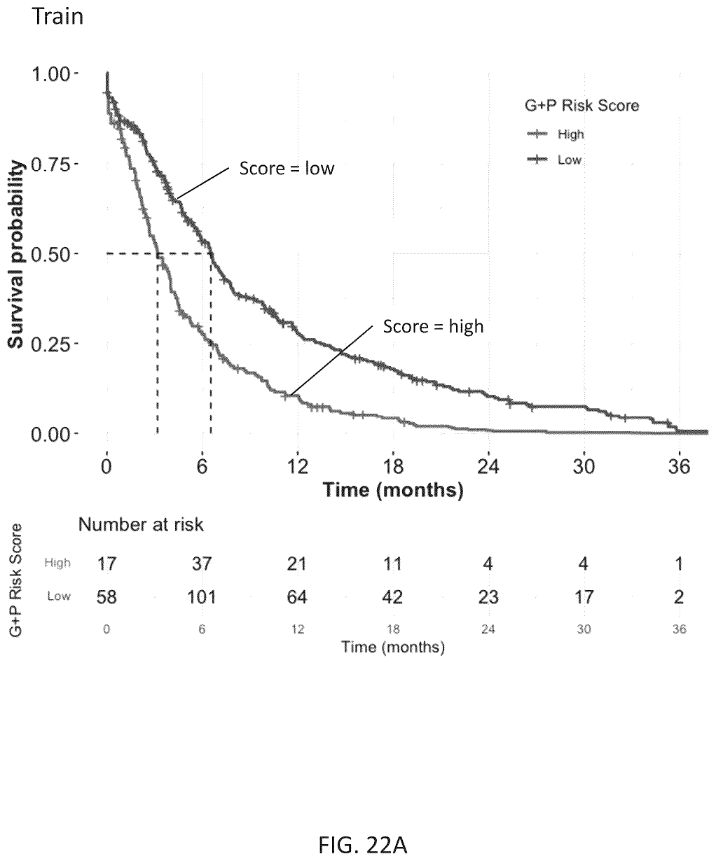

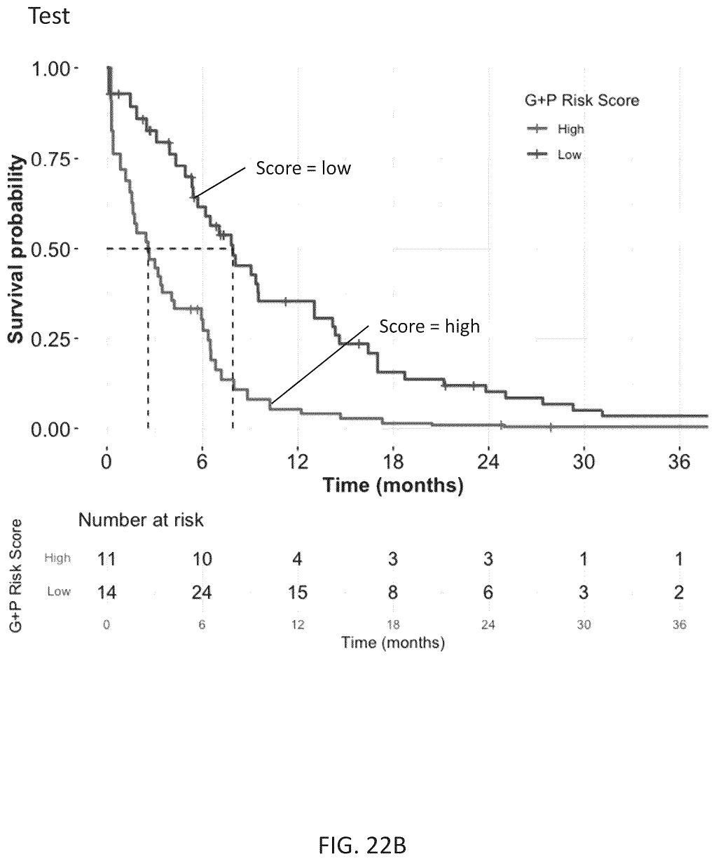

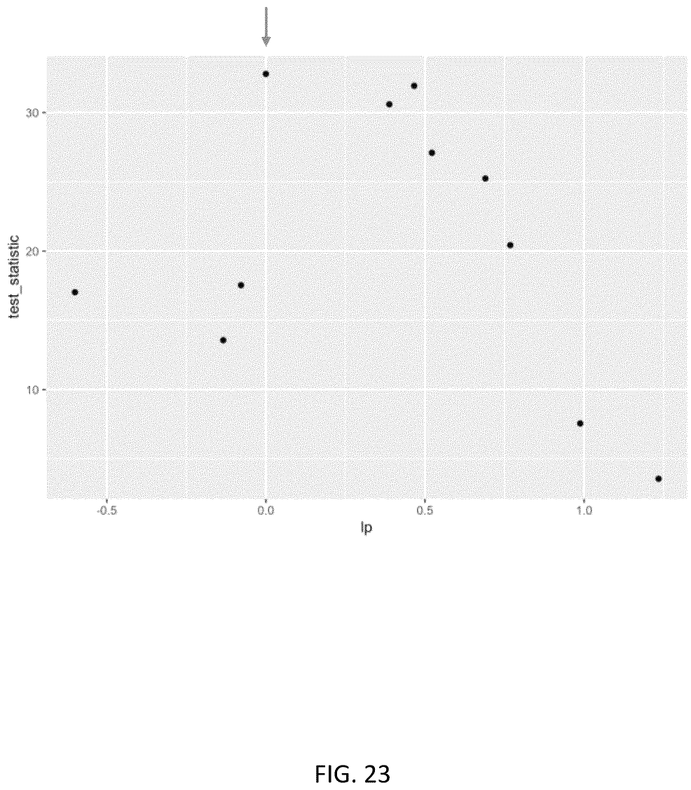

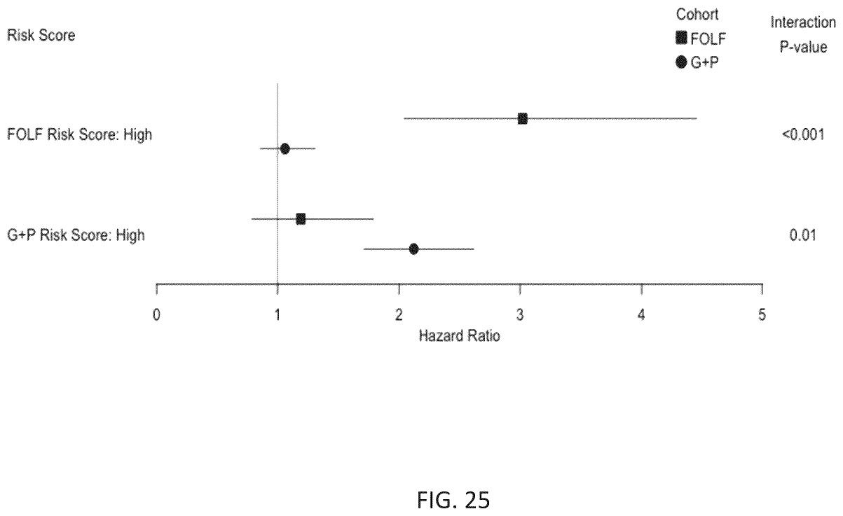

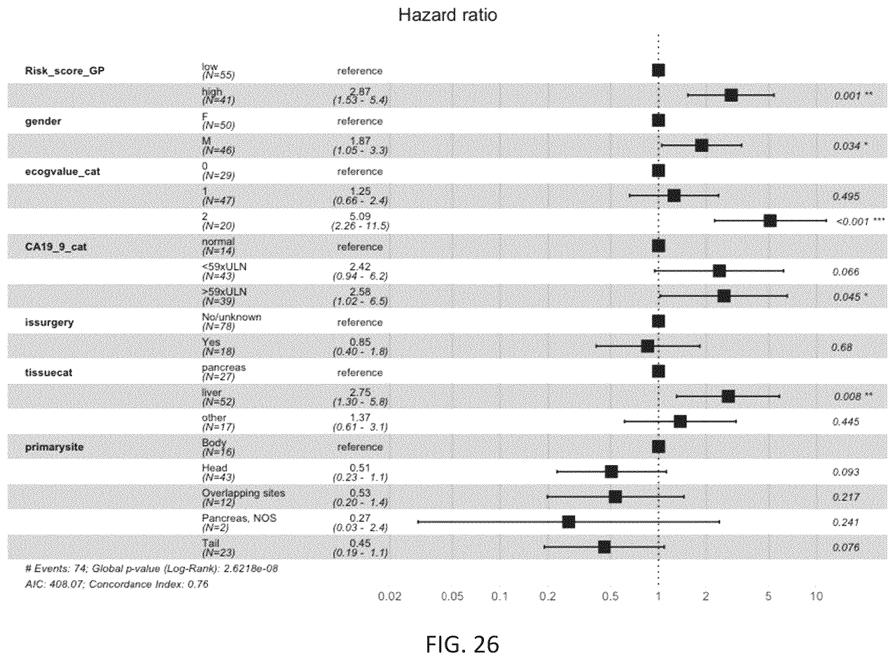

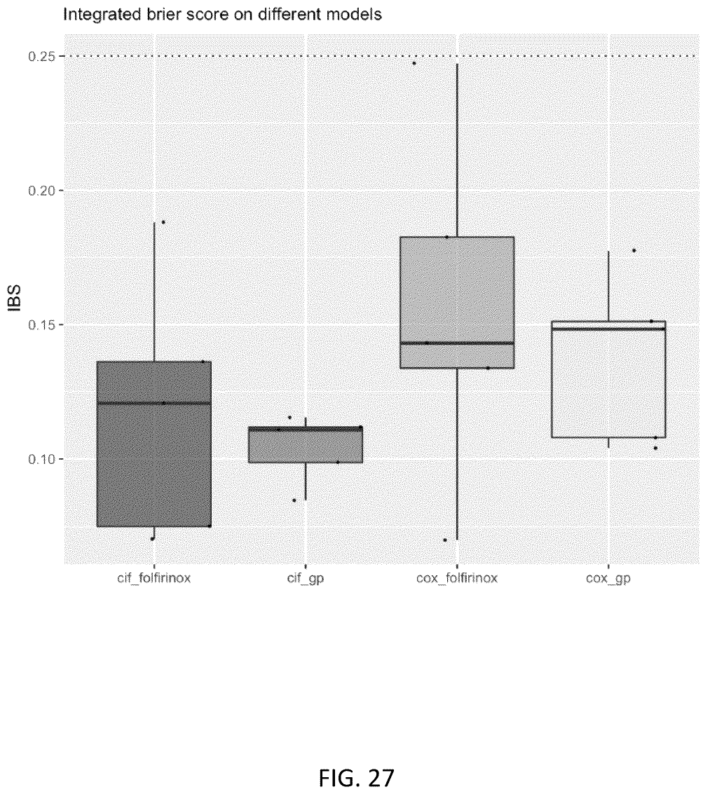

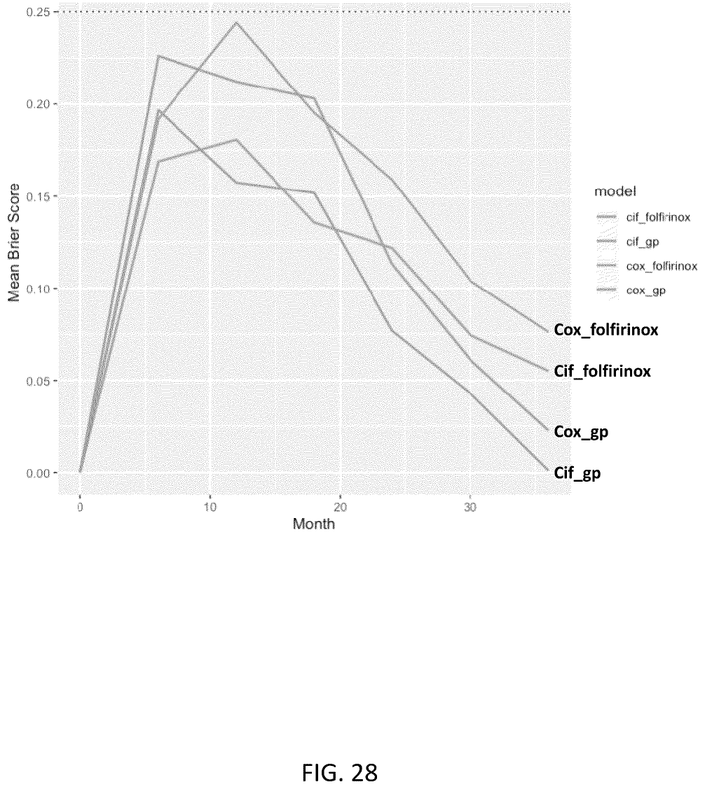

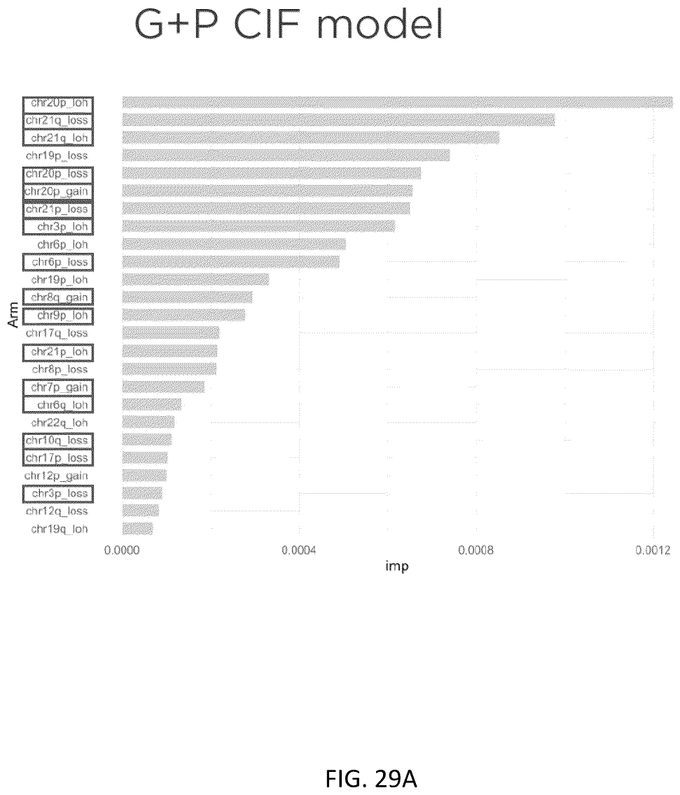

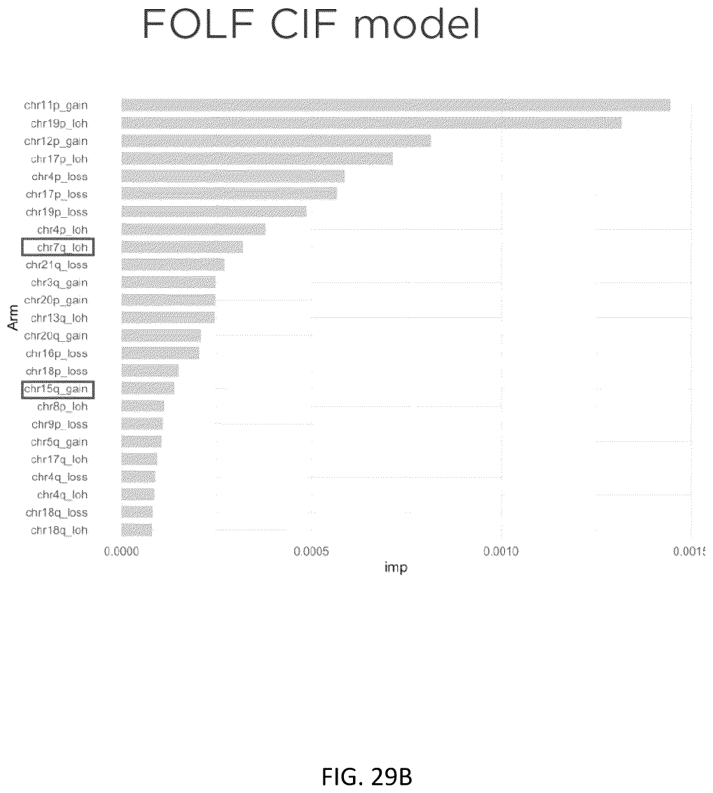

A multivariable Cox regression model was used to calculate a risk score for each treatment arm based on chromosome arm level aneuploidies and clinical features associated with patient survival (FIGS. 11-16). A multivariable Cox regression model was used to determine risk scores based solely on chromosome arm level aneuploidies associated with survival (FIGS. 17-24). Additionally, the conditional inference survival forest (CIF) model outperformed the multivariable Cox regression model on predicting survival in FOLF-treated and G+P-treated patients based on chromosome arm level aneuploidy data (FIGS. 25-29).

### CONCLUSIONS

Chromosome arm-level aneuploidies associated with survival for FOLF and G+P regimens were identified. These results highlight the value of an aneuploidy-based risk score in predicting response and choosing a first-line treatment, such as FOLFIRINOX or gemcitabine plus paclitaxel.

### Example 2-Identification of Cytoband-Level Aneuploidy Biomarkers Associated with Response to G+P Treatment in Metastatic Pancreatic Cancer

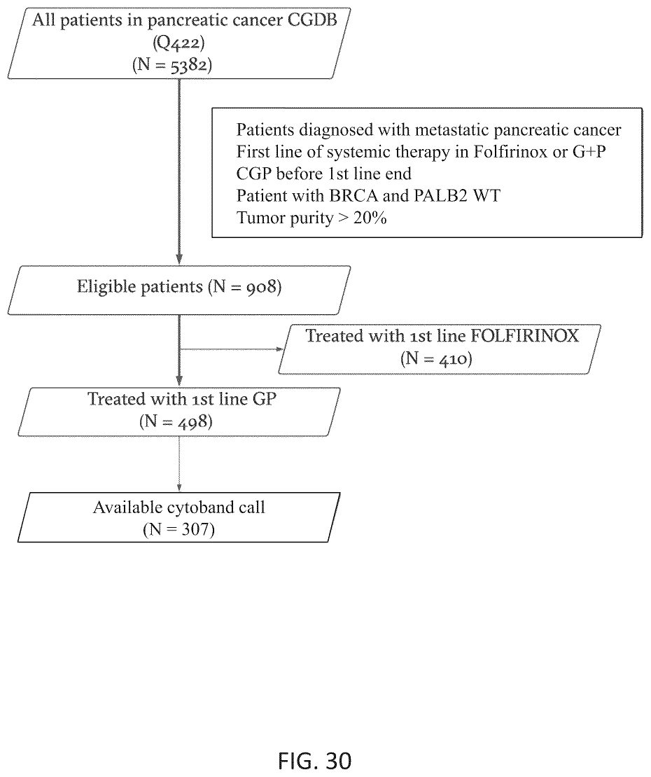

FIG. 30 provides a non-limiting example of a study design for identifying associations between cytoband level aneuploidy data and patient survival in a metastatic pancreatic cancer patient cohort treated with gemcitabine plus albumin-bound paclitaxel (G+P, also referred to as GP). A cohort of 5,382 pancreatic cancer patients was filtered to identify the subset of pancreatic cancer patients (908 patients) who had been diagnosed with metastatic pancreatic cancer, had undergone comprehensive genomic profiling prior to the end of first line treatment, were wild-type for the BRCA and PALB2 genes, had provided samples with a tumor purity of greater than 20%, and who had undergone first line treatment with either the Folfirinox or G+P treatment regimens. The resulting cohort of metastatic pancreatic cancer patients treated with Folfirinox comprised 410 patients, while the cohort of metastatic pancreatic cancer patients treated with GP comprised 498 patients. Of the latter, a cohort of 307 patients were subjected to cytoband analysis.

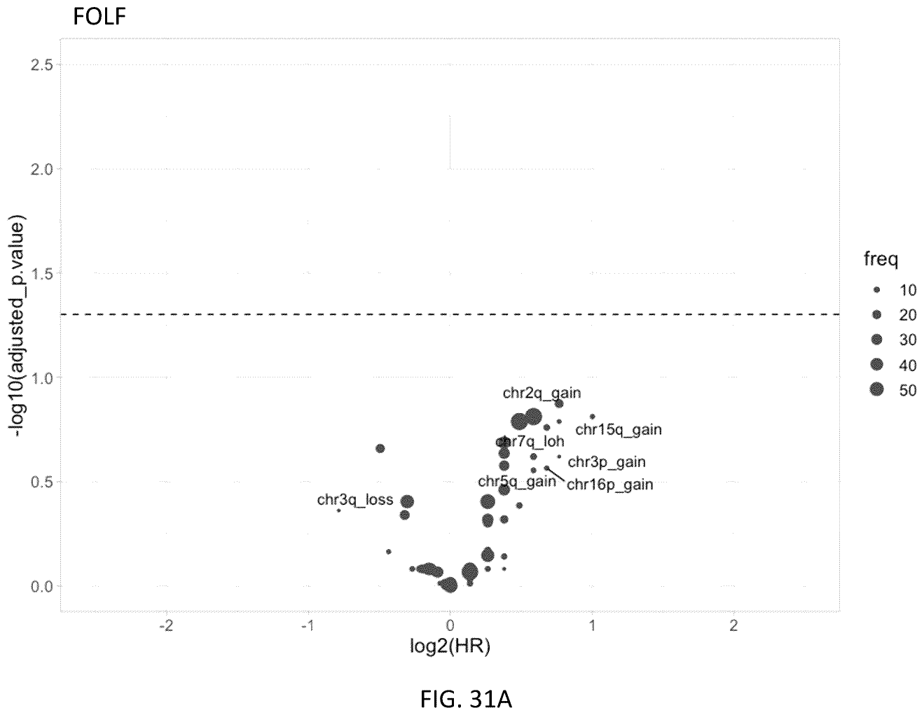

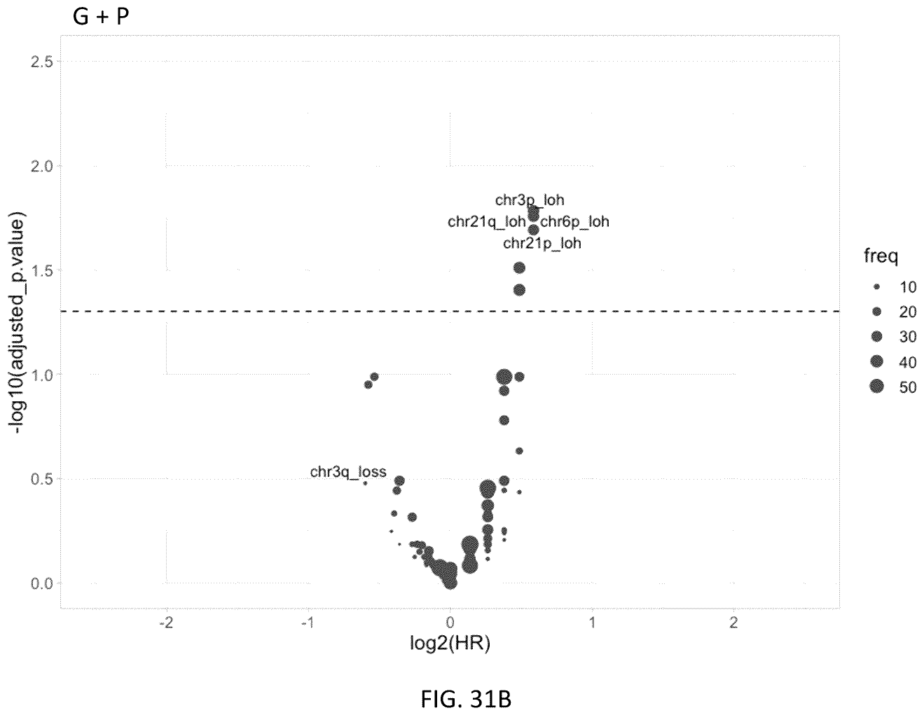

FIGS. 31A-B provide non-limiting examples of plots of adjusted p value (e.g., the smallest significance level at which the comparison is statistically significant as part of multiple comparison testing) versus hazard ratio (HR) for cohorts of metastatic pancreatic cancer patient treated with either FOLF or G+P that demonstrate associations between chromosome arm level aneuploidy data and survival in each cohort. FIG. 31A: data for the FOLF cohort. FIG. 31B: data for the G+P cohort. As indicated, the size of the symbols indicates the relative frequency of occurrence of copy number gain, copy number loss, or loss of heterozygosity (Loh) for the specified chromosomal regions.

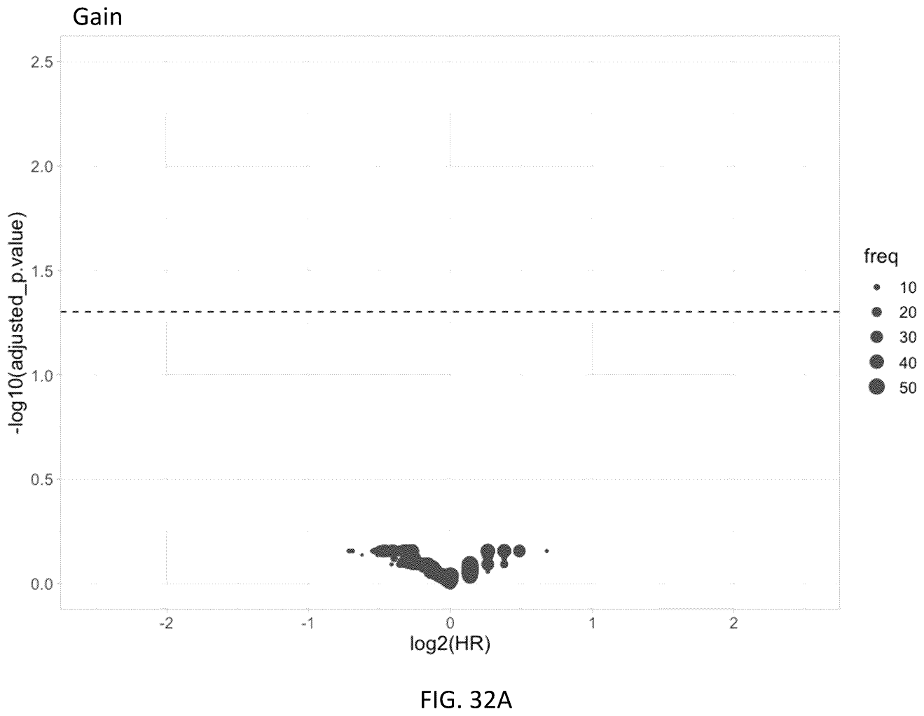

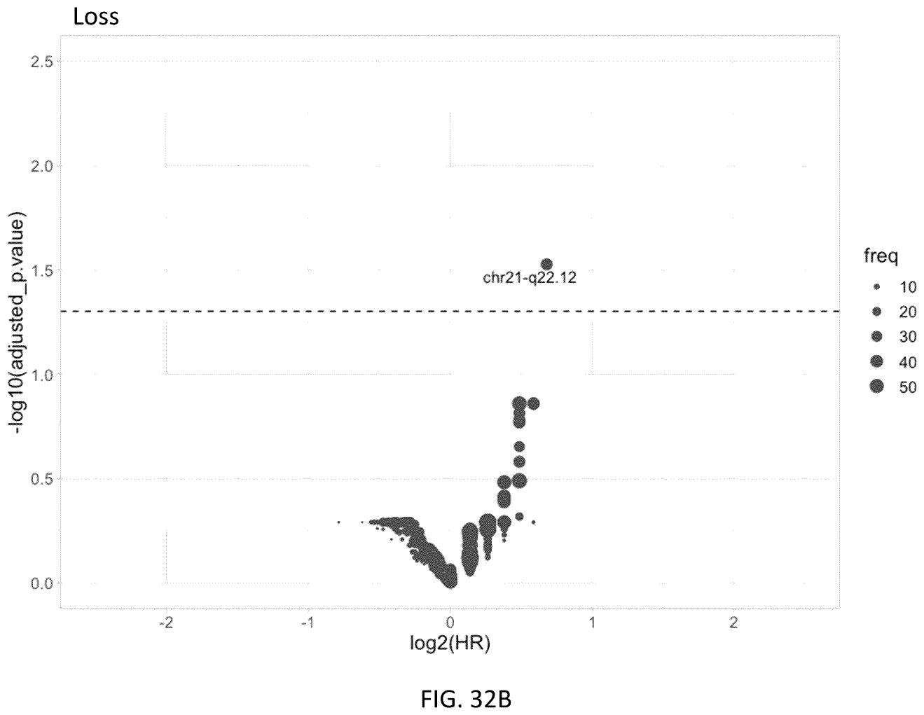

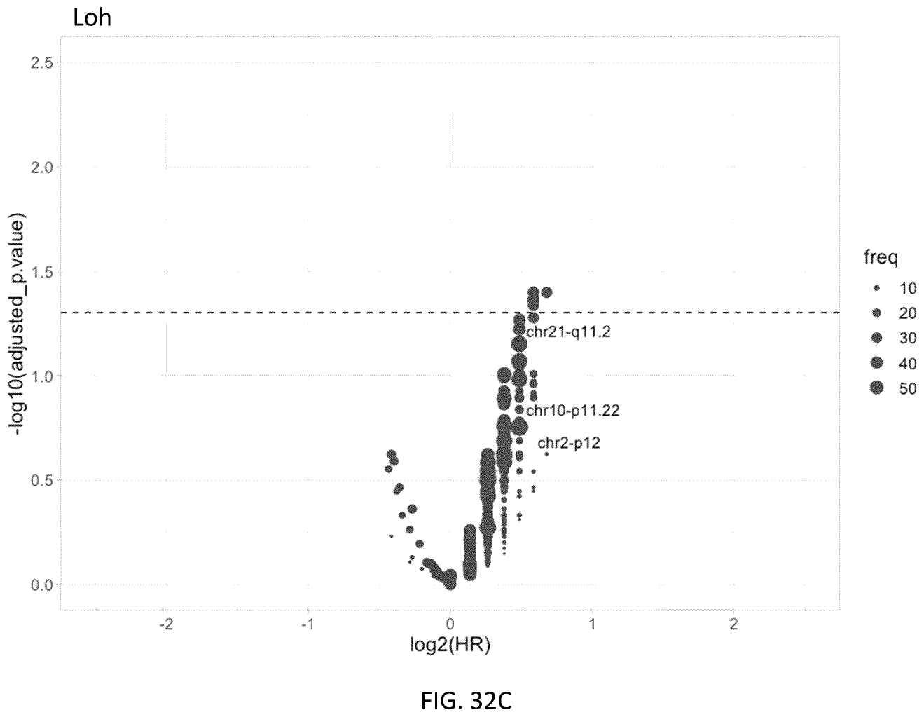

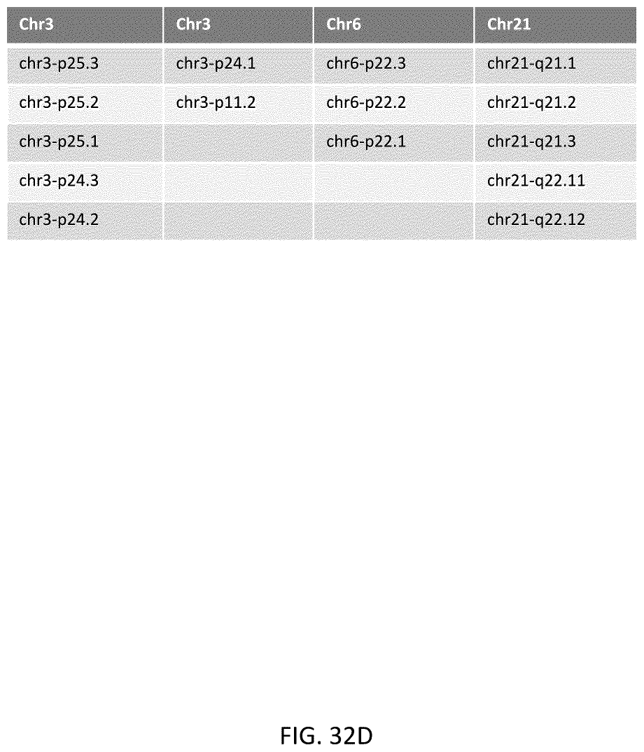

FIGS. 32A-D provide non-limiting examples of plots of copy number gain, copy number loss, and loss of heterozygosity (Loh) data that demonstrate associations between cytoband level aneuploidy data and survival in a GP-treated cohort of metastatic pancreatic cancer patients. FIG. 32A: copy number gain data. FIG. 32B: copy number loss data. FIG. 32C: loss of heterozygosity (Loh) data. FIG. 32D: summary of chromosome regions that may exhibit chromosome alterations comprising loss of heterozygosity. As indicated, the size of the symbols indicates the relative frequency of occurrence of copy number gain, copy number loss, or loss of heterozygosity (Loh) for the specified chromosomal regions. The data indicate that certain cytoband losses or Loh (e.g., the loss of chr21.q22.12) are associated with survival benefit for the GP-treated cohort (see Table 1).

TABLE 1
Non-limiting examples of cytobands associated with
survival benefit for the GP-treated cohort.

| Alteration | Cytoband |
|---|---|
| Loh | chr21-q22.12 |
| Loh | chr3-p11.2 |
| Loh | chr3-p25.1 |
| Loh | chr6-p22.1 |
| Loh | chr6-p22.2 |
| Loh | chr6-p22.3 |
| Loh | chr21-q21.1 |
| Loh | chr21-q21.2 |
| Loh | chr21-q21.3 |
| Loh | chr21-q22.11 |
| Loh | chr3-p24.1 |
| Loh | chr3-p24.2 |
| Loh | chr3-p24.3 |
| Loh | chr3-p25.3 |
| Loh | chr3-p25.2 |
| Loss | chr21-q22.12 |

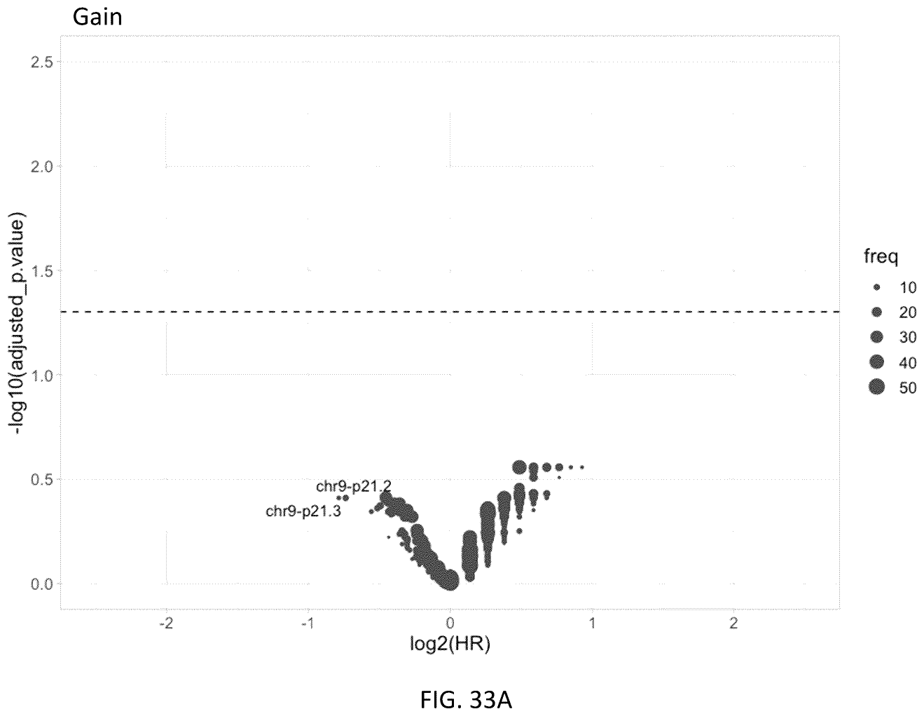

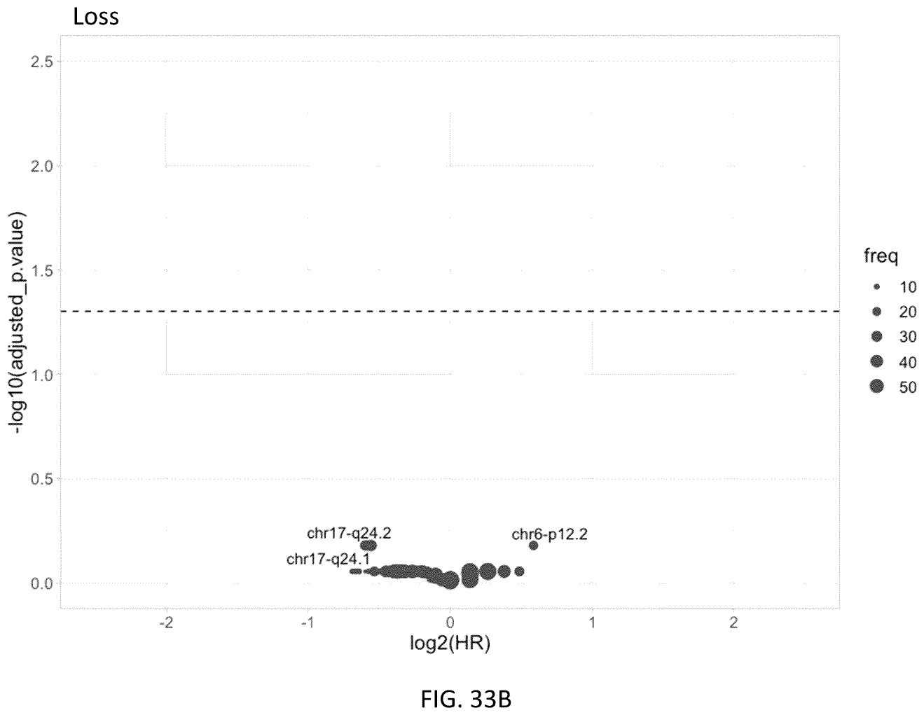

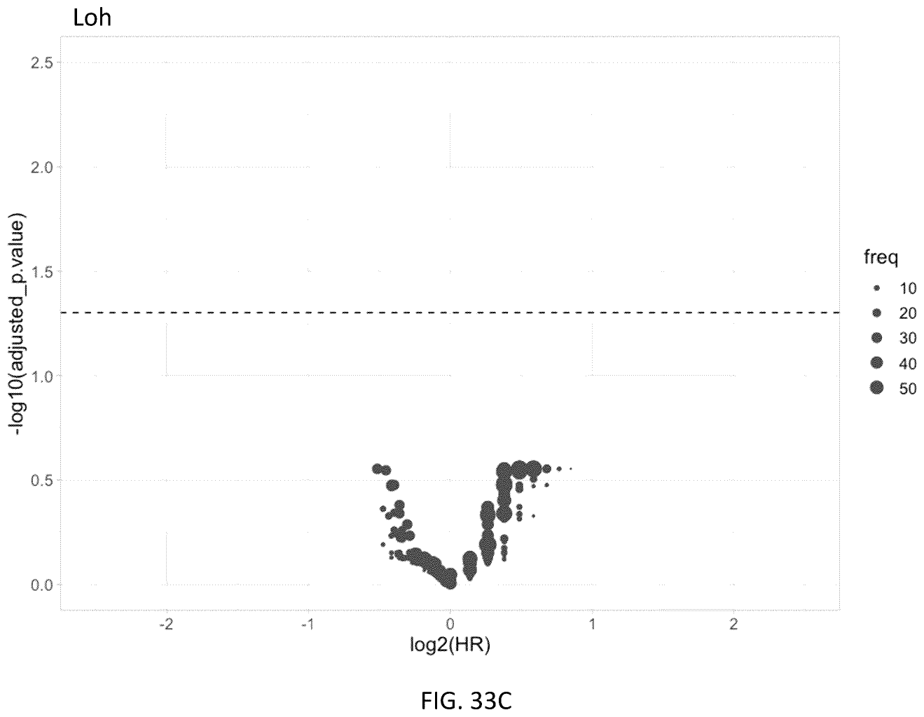

FIGS. 33A-C provide non-limiting examples of plots of copy number gain, copy number loss, and loss of heterozygosity (Loh) data that demonstrate associations between cytoband level aneuploidy data and survival in a FOLF-treated cohort of metastatic pancreatic cancer patients. FIG. 33A: copy number gain data. FIG. 33B: copy number loss data. FIG. 33C: loss of heterozygosity (Loh) data. As indicated, the size of the symbols indicates the relative frequency of occurrence of copy number gain, copy number loss, or loss of heterozygosity (Loh) for the specified chromosomal regions.

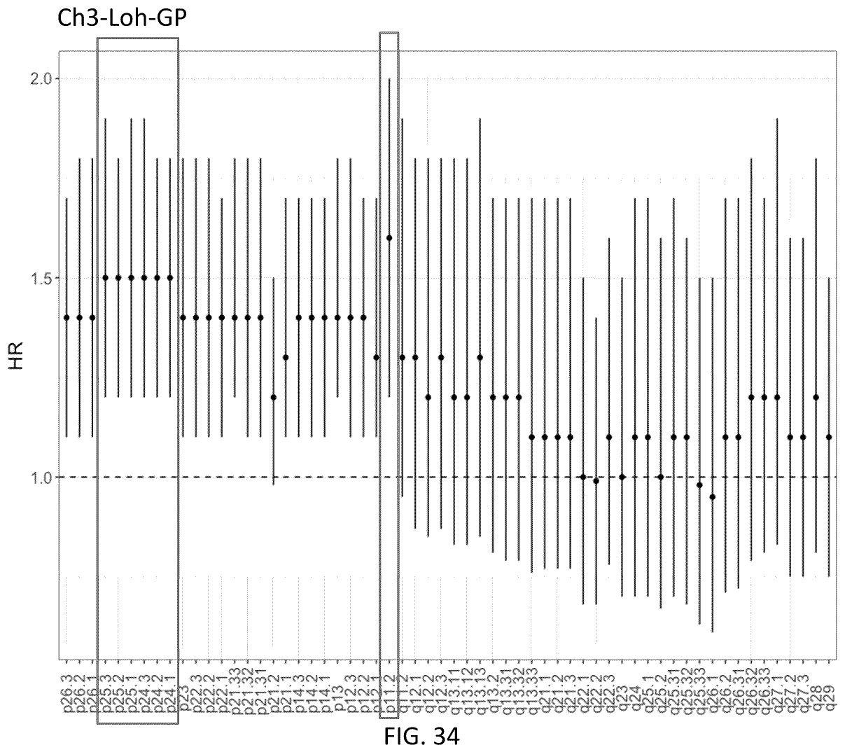

FIG. 34 provides a non-limiting example of hazard ratio (HR) data (mean and 95% confidence interval) plotted for loss of heterozygosity in different regions of chromosome 3 for a cohort of metastatic pancreatic cancer patients treated with G+P. The boxes illustrate the hazard ratio data for deletions in the 3p25.3-p24.1 region and the p11.2 region of chromosome 3, the two chromosome regions for which loss of heterozygosity exhibited the highest hazard ratios. Deletions at 3p25.3-p23 is frequently encountered in endocrine pancreatic tumors and is associated with metastatic progression, and thus provide a novel pancreatic endocrine tumor suppressor gene locus on chromosome 3p with clinical prognostic implications.

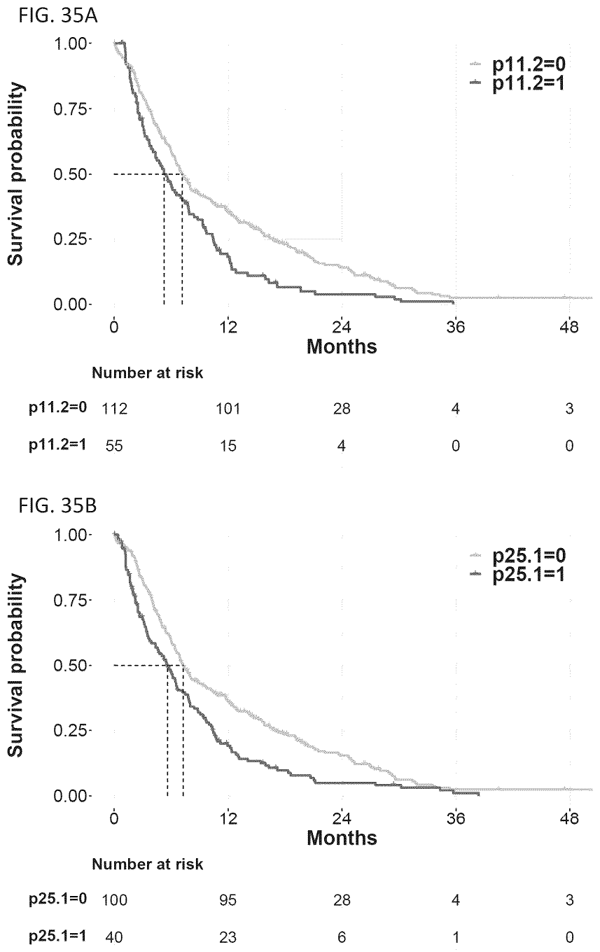

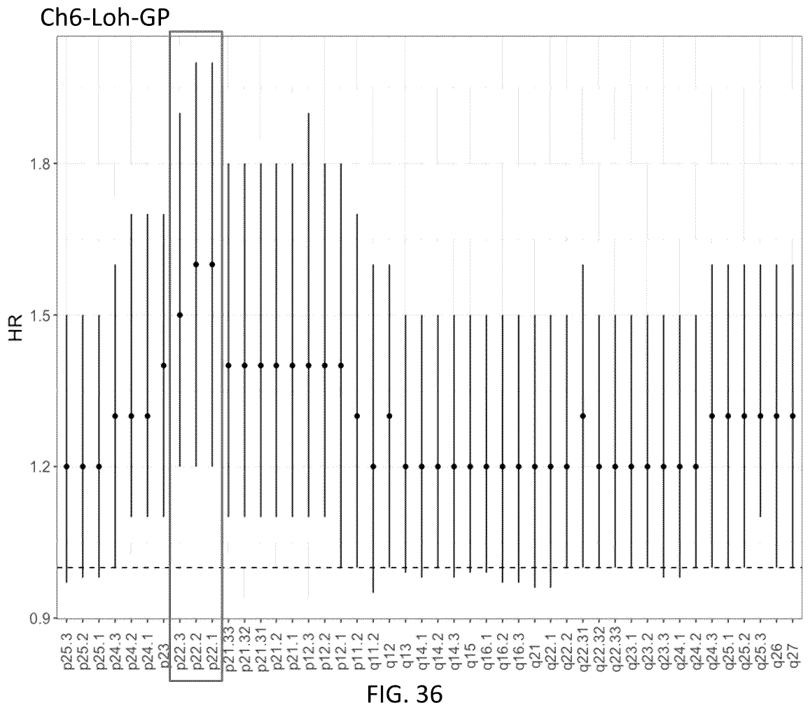

FIGS. 35A-B provide non-limiting examples of survival plots for a cohort of metastatic pancreatic cancer patients treated with G+P. The tables below the plots indicate the corresponding numbers of patients represented by the survival data. FIG. 35A: survival data for metastatic pancreatic patients exhibiting loss of heterozygosity at chromosome region 3.p11.2 (p11.2=1) compared to that for patients with no loss of heterozygosity at 3.p11.2 (p11.2=0). FIG. 35B: survival data for metastatic pancreatic patients exhibiting loss of heterozygosity at chromosome region 3.p25.1 (p25.1=1) compared to that for patients with no loss of heterozygosity at 3.p25.1 (p25.1=0).

FIG. 36 provides a non-limiting example of hazard ratio (HR) data (mean and 95% confidence interval) plotted for loss of heterozygosity in different regions of chromosome 6 for a cohort of metastatic pancreatic cancer patients treated with G+P. The box indicates the hazard ratio data for loss of heterozygosity in the p22.3-p22.1 region of chromosome 6.

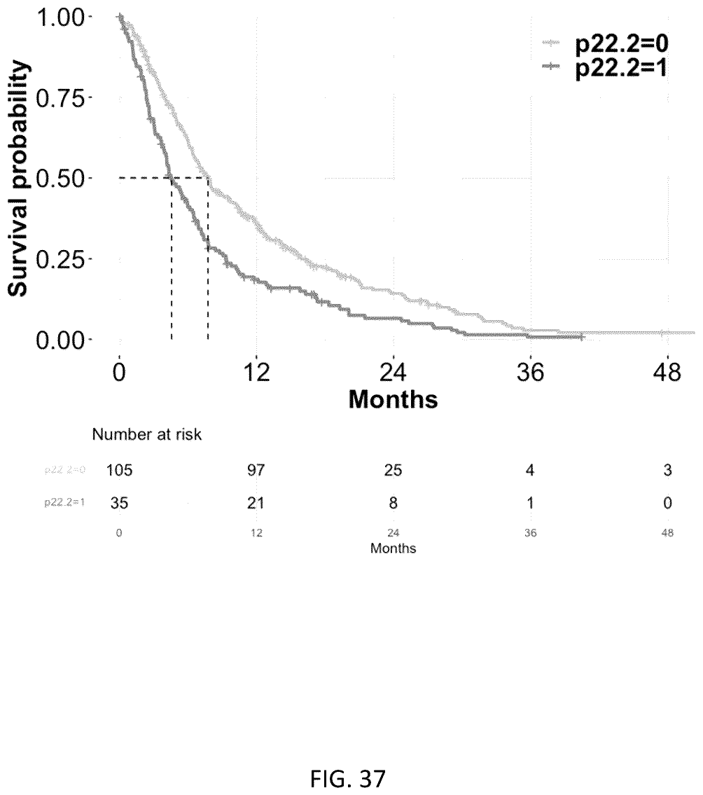

FIG. 37 provides a non-limiting example of a survival plot for a cohort of metastatic pancreatic cancer patients treated with G+P. The plot shows survival data for metastatic pancreatic patients exhibiting loss of heterozygosity at chromosome region 6.p22.2 (p22.2=1) compared to that for patients with no loss of heterozygosity at 6.p22.2 (p22.2=0). The table below the plot indicates the corresponding numbers of patients represented by the survival data.

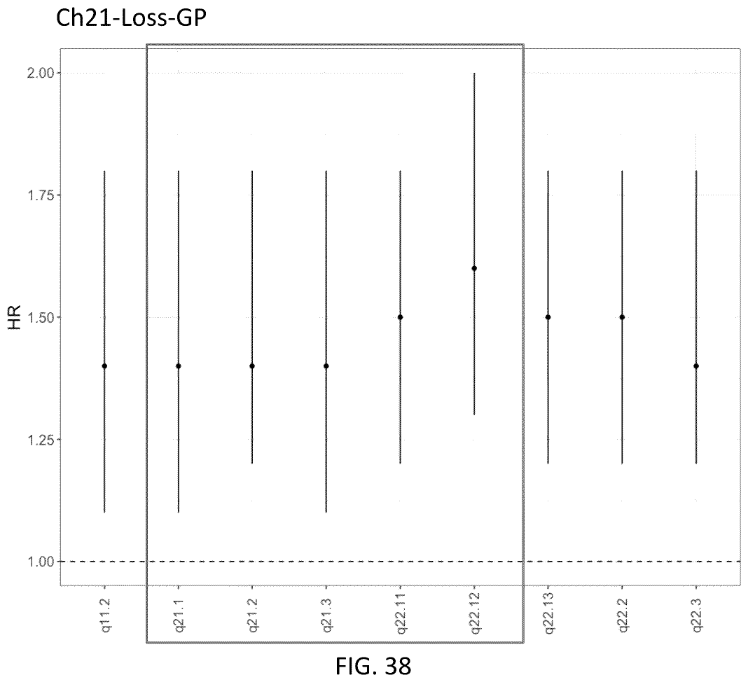

FIG. 38 provides a non-limiting example of hazard ratio (HR) data plotted for copy number loss in different regions of chromosome 21 for a cohort of metastatic pancreatic cancer patients treated with G+P. The box indicates hazard ratio data for copy number losses in the q21.1 to q22.12 region of chromosome 21.

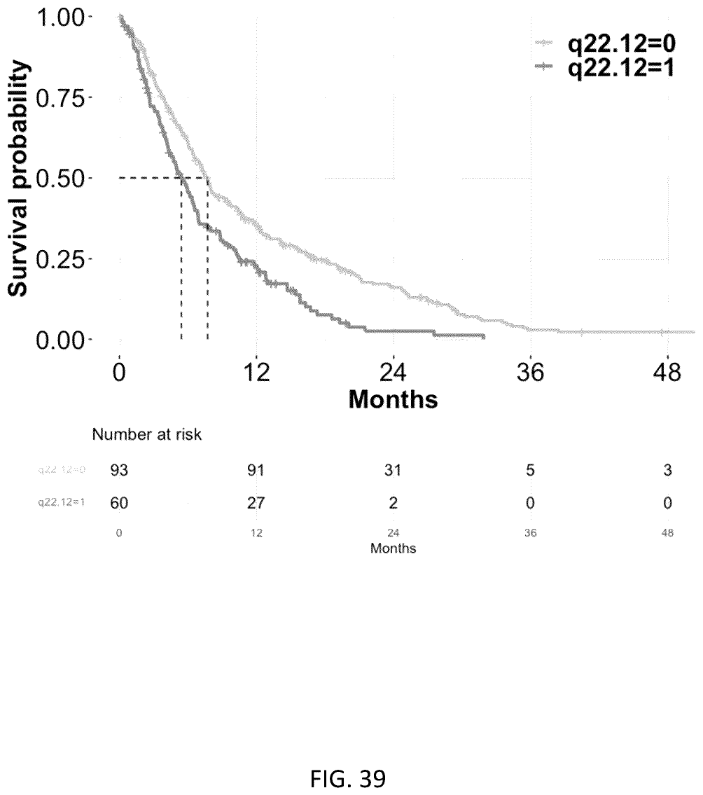

FIG. 39 provides a non-limiting example of a survival plot for a cohort of metastatic pancreatic cancer patients treated with G+P. The plot shows survival data for metastatic pancreatic patients exhibiting copy number loss at chromosome region 21.922.12 (q22.12=1) compared to that for patients with no copy number loss at 21.q22.12 (q22.12=0). The table below the plot indicates the corresponding numbers of patients represented by the survival data.

Overall, the study identified aneuploidies at the chromosome cytoband level that were associated with survival outcomes for patients treated with the G+P regimen. These findings underscore the significance of using an aneuploidy-based approach in predicting treatment response and selecting optimal first-line therapy for individuals with metastatic pancreatic cancer.

It should be understood from the foregoing that, while particular implementations of the disclosed methods and systems have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

1. A method of treating a subject having a cancer with a first anti-cancer therapy comprising (a) determining a risk score for the subject based on aneuploidy status or loss of heterozygosity (LOH) for one or more subgenomic intervals in a sample from the subject, and (b) treating the subject with the first anti-cancer therapy if the risk score is less than a predetermined threshold.

2. A method of selecting a first anti-cancer therapy for a subject having a cancer, the method comprising determining a risk score for the subject based on aneuploidy status or loss of heterozygosity (LOH) for one or more subgenomic intervals in a sample from the subject, wherein if the risk score is less than a predetermined threshold, the subject is identified as one who may benefit from treatment with the first anti-cancer therapy.

3. A method of identifying a subject having a cancer for treatment with a first anti-cancer therapy comprising (a) determining a risk score for the subject based on aneuploidy status or loss of heterozygosity (LOH) for one or more subgenomic intervals in a sample from the subject, and (b) identifying the subject for treatment with the first anti-cancer therapy if the risk score is less than a predetermined threshold.

4. The method of claim 1, wherein the first anti-cancer therapy is a chemotherapy or an immune-oncology (IO) therapy.

5. The method of claim 1, wherein the first anti-cancer therapy comprises FOLFIRINOX, gemcitabine plus albumin-bound paclitaxel, gemcitabine, capecitabine, fluorouacil plus irinotecan liposomal and leucovorin, FOLFIRI, or capecitabine plus gemcitabine.

6. The method of claim 1, wherein the risk score is calculated by a method comprising:
obtaining genomic data comprising aneuploidy status or loss of heterozygosity data for one or more subgenomic intervals in a sample from the subject;
analyzing the genomic data for the subject using a model configured to receive genomic data comprising aneuploidy status or loss of heterozygosity (LOH) data for the one or more subgenomic intervals identified in the subject and output a risk score for the subject, wherein the aneuploidy or LOH data are associated with a patient survival metric, and wherein the risk score predicts a response to a selected treatment for the subject.

7. The method of claim 6, further comprising converting the risk score output by the model for the subject to a binary (high-low) risk score based on a comparison to the predetermined threshold.

8. The method of claim 7, wherein the predetermined threshold is defined by a risk score value that maximizes a log-rank statistic for risk scores calculated for a patient cohort used to train the model.

9. The method of claim 7, wherein a low risk score indicates that the subject is likely to survive longer than a subject with a high risk score if treated with the selected treatment.

10. The method of claim 6, wherein the genomic data is based on sequence read data derived from a comprehensive genomic profiling assay.

11. The method of claim 6, wherein analyzing the genomic data for the subject further comprises analysis of clinical feature data for the subject.

12. The method of claim 6, wherein analyzing the genomic data for the subject further comprises analysis of Eastern Cooperative Oncology Group (ECOG) performance data for the subject.

13. The method of claim 6, wherein the model is a machine learning model.

14. The method of claim 13, wherein the machine learning model comprises a multivariable Cox proportional hazards regression model or a conditional inference forest model.

15. The method of claim 6, wherein the risk score is for treatment with FOLFIRINOX, and the one or more subgenomic intervals for which aneuploidy or LOH correlated with a patient survival metric comprise Chr3q, Chr4p, Chr5p, Chr5q, Chr7q, Chr11p, Chr12p, Chr12q, Chr15q, Chr16p, Chr17p, Chr19p, Chr19q, Chr20p, Chr22q, or any combination thereof.

16. The method of claim 6, wherein the risk score is for treatment with gemcitabine plus albumin-bound paclitaxel, and the one or more subgenomic intervals for which aneuploidy or LOH correlated with a patient survival metric comprise Chr1p, Chr1q, Chr3p, Chr6p, Chr6q, Chr7p, Chr7q, Chr8q, Chr9p, Chr9q, chr14q, Chr15q, Chr16p, chr17p, Chr17q, Chr18q, Chr19p, Chr20p, Chr21p, Chr21q, Chr22q, or any combination thereof.

17. The method of claim 6, wherein the patient survival metric comprises a hazard ratio, a progression free survival, an overall survival, a disease-free survival, an objective tumor response rate, a time to tumor progression, a time to treatment failure, a durable complete response, a time to next treatment, or any combination thereof.

18. The method of claim 1, wherein the sample comprises a tissue biopsy sample or a liquid biopsy sample.

19. The method of claim 1, wherein the cancer is pancreatic cancer.

20. The method of claim 19, wherein the pancreatic cancer is metastatic pancreatic cancer.
